(19) United States
(12) Patent Application Publication
(10) Pub. No.: US 2009/0088432 A1
(43) Pub. Date: Apr. 2, 2009

(54) Thiazolinones and Oxazolinones and their Use as Ptp1b Inhibitors

(76) Inventors: Rakesh Kumar Banerjee, Gujarat (IN); Ramesh Chandra Gupta, Gujarat (IN); Davinder Tuli, Gujarat (IN); Milind Rode, Gujarat (IN); Bharat Suthar, Gujarat (IN); Dhananjay Umrani, Gujarat (IN); Padmaja Pathak, Gujarat (IN); Tejal Choksi, Gujarat (IN); Anita Chaudhary, Gujarat (IN)

Correspondence Address: JACOBSON HOLMAN PLLC, 400 SEVENTH STREET N.W., SUITE 600, WASHINGTON, DC, 20004, US

(21) Appl. No.: 11/992,016

(22) PCT Filed: Sep. 15, 2006
(86) PCT No.: PCT/IN2006/000368

§ 371 Date: Aug. 13, 2008

(30) Foreign Application Priority Data
Sep. 16, 2005 (IN) 860/KOL/2005

(51) Int. Cl. A61K 31/5377; C07D 277/38; C07D 413/02; A61K 31/426

(52) U.S. Cl. 514/236.8; 548/194; 514/370; 544/133

(57) ABSTRACT

The present invention encompasses; the novel substituted heterocyclic compounds represented by formula (I) or their pharmaceutically acceptable salts, wherein the substituents in formula (I) have the meanings as defined in the specification. The invention also encompasses process for preparing such compound, pharmaceutical compositions and methods of treating or preventing PTP-1B mediated diseases.

### BACKGROUND OF THE INVENTION

1. Field of the Invention

The present invention is directed to a novel substituted heterocyclic compounds or pharmaceutically acceptable salts thereof. Present invention also relates to process for their preparation and pharmaceutically acceptable compositions containing them and their application as pharmaceuticals for treatment of diseases.

The said compounds by virtue of its inhibitory action on an enzyme protein tyrosine phosphatase 1B (PTP1B) are useful in the treatment of diabetes and obesity.

2. Description of the Related Art

Type 2 diabetes (T2DM), also known as non-insulin dependent diabetes mellitus (NIDDM) afflicts about 150 million people worldwide and the numbers continue to grow each year. Similarly the incidence and prevalence of obesity is also increasing at alarming rate. Obesity is also associated with heart disease, diabetes, stroke, high blood pressure and some cancers. The increase in the prevalence of obesity has been identified as a major cause of the projected increase in diabetes. The increase in type 2 diabetes and obesity was previously observed primarily in adult population, associated with a sedentary lifestyle, but has now become a medical problem in children also (Sinha R. et al, NEJM 346; 802-10; 2002 & Rocchini A P., NEJM 346; 854-55; 2002) The increased incidence of type 2 diabetes and obesity in the population has fueled an intensified search for new therapeutic treatment options.

The relationship of obesity and type 2 diabetes has a phylogenetic component and is associated with insulin resistance. Type 2 diabetes and obesity are characterised by resistance to hormones, insulin and leptin, possibly due to attenuated or diminished signaling from the receptors. (Kishor M. Wasan & Norbert A. Looije; J Pharm Pharmaceut Sci (www.cspscanada.org) 8(2):259-271, 2005)

In an attempt to maintain glucose homeostasis in the face of progressive insulin resistance, the pancreas secretes increasingly higher amounts of insulin. However, when the β-cells no longer can secrete adequate amount of insulin, hyperglycemia and type 2 diabetes develop. Insulin resistance plays an important role in the development of abnormalities such as impaired glucose tolerance, type 2 diabetes, obesity and hyperlipidemia. Since defective insulin signaling has been found as one of the root cause of insulin resistance, therapeutic strategies designed to improve insulin receptor signaling have generated special interest for the researchers.

Presently available therapies for Type 2 Diabetes include acarbose, sulfonylureas, biguinides, thiazolidinediones and insulin therapy. These therapies address different metabolic defects present in type 2 diabetic conditions. Unfortunately none of these are capable of addressing multiple defects. These therapies also have their own limitations. Sulfonylureas risk hypoglycemia and weight gain, whereas acarbose has G.I. side effects. Biguinides also causes G.I. side effects and lactic acidosis and according to reports, it has limitation of having effective in restricted populations. The much sought after thiazolidinediones (TZDs) have multiple drawbacks like weight gain, edema, nausea, poor responder rate and effectiveness in restricted populations. The ultimately used insulin therapy poses problems of route of administration, hypoglycemia and weight gain. Thus, in the presently available therapies for diabetes, none has a potential to address insulin signaling.

Hence, there is a need for oral therapy for Type 2 Diabetes that will improve insulin signaling and will also address the commonly present side effects of hypoglycemia and weight gain.

The reversible tyrosine phosphorylation of key proteins by protein tyrosine kinases (PTKs) and protein tyrosine phosphatases (PTPs) is one of the major processes involved in the regulation of metabolic and mitogenic signal transduction processes in cells (Moller et al, 2000). The insulin receptor (IR) is a classical example of this. On binding insulin, the resultant conformational changes allow the IR to autophosphorylate itself on defined tyrosine residues and thereby initiate its activation cascade (Hubbard, 1997). PTPs down regulate signal transduction pathways by dephosphorylating the tyrosine residues on PTKs, including the IR, and other downstream signaling proteins. Therefore, selective inhibition of critical PTPs has been proposed as a means whereby signaling pathway activation may be maintained or even initiated and thus such agents could be useful in the treatment of diseases in which an enhanced or prolonged signaling is warranted (Taylor and Hill, 2004).

Among the various candidate targets for obtaining insulin sensitizers, protein tyrosine phosphatases (PTPs) are particularly interesting since they play a key role in modulating the activity of the insulin signaling cascade. Several candidate PTPs have been identified, including PTP1B, PTPα, leukocyte antigen related (LAR) and T cell protein tyrosine phosphatase (TCPTP). PTP1B has been of particular interest since it seems to be a key regulator of insulin receptor activity that acts at the insulin receptor and at downstream signaling components, such as IRS1.

PTP1B has been implicated as a negative regulator of the insulin signaling pathway in vitro. Experimental evidence has also shown that the mice null for this protein have increased glucose tolerance and insulin sensitivity, together with resistance to diet-induced obesity (Klaman et al, 2000).

Knock out studies have been carried out to understand the importance of PTP1B in the overall physiology of insulin signaling and glucose metabolism (Kiaman et al, 2000, Elchebly et al, 1999). The PTP1B knockout mouse showed many characteristics that are considered desirable for an antidiabetic treatment. Importantly the knockout mice grew normally. These animals had lower blood glucose and insulin levels as well as the consequent marked increase in insulin sensitivity. Moreover, the insulin stimulated tyrosine phosphorylation levels of insulin receptor, and IRS-1 were increased/prolonged in muscle and liver. This is in contrast to the main target tissue for the PPARγ agonist class of insulin sensitizers, which is adipose tissue. These animals showed decreased plasma triglycerides too. However, most remarkably, the knockout animals also exhibited a resistance to weight gain when placed on a high fat diet. They had lower body fat. Insulin is an anabolic hormone and insulin administration is generally associated with weight gain rather than weight loss. A number of recent studies have concluded that PTP1B is involved in the dephosphorylation of JAK2, which is an important second messenger of the leptin receptor. Hence, resistance to weight gain might be due to improved leptin action (Cheng et al, 2002).

The pharmacological effects of PTP1B inhibition were studied by treatment of genetically diabetic mice with PTP1B antisense construct. Intraperitoneal administration of highly selective second generation PTP1B ASO to db/db and ob/ob mice once a week for 4 weeks caused significant reduction in blood glucose levels to near normal values. Importantly hypoglycemia was not observed. The decreases in blood glucose were accompanied by attenuation of hyperinsulinemia in ob/ob mice. These results, along with the improved performance of the treated diabetic mice on glucose and insulin tolerance tests, suggest that the ASO acts as an insulin sensitizer. Additionally, in agreement with the studies in the PTP1B knockout mice, significantly reduced weight gain was seen in fat fed normal mice treated with the PTP1B ASO (Gum et al, 2003; Rondinone et al, 2002; Zinker et al, 2002).

Thus, the scientific data obtained from PTP1B knockout and PTP1B inhibition studies clearly indicate that PTP1B inhibition improves insulin sensitivity by enhancing insulin signaling. They have utility in controlling or treating type 1 or type 2 diabetes, in improving glucose tolerance and decreasing insulin resistance. These inhibitors can also be of use in treating diet-induced obesity by improving leptin signaling and function.

Furthermore, there is evidence that suggests inhibition of protein tyrosine phosphatase PTP1B is therapeutically beneficial for the treatment of diseases such as, autoimmune disease, acute and chronic inflammation, osteoporosis and various forms of cancer (J. Natl. Cancer Inst. 86: 372-378 (1994); Mol. Cell. Biol. 14: 6674-6682 (1994); The EMBO J., 12: 1937-1946 (1993); J. Biol. Chem. 269: 30659-30667 (1994); and Biochemical Pharmacology 54: 703-711 (1997)).

Because of the important roles played by upregulated protein tyrosine phosphatase PTP1B in the disease states of T2DM, obesity, autoimmune disease, acute and chronic inflammation, osteoporosis and various forms of cancer, agents that inhibit this enzyme specifically may provide the desired therapeutic benefits without the unwanted side effects derived from inhibiting the related phosphatases.

Accordingly there is a continuing need to identify novel compounds, which are PTP1B inhibitors. It has been found that the compounds of the present invention have been categorically shown to inhibit the enzyme PTP1B and accordingly have value in the treatment of disease conditions mediated by the PTP1B enzyme.

WO2004047760, WO2005082901 discloses certain thiazolidine compounds and its method for preparation that are hYAk3 inhibitors, useful for the treatment of diseases associated with imbalance or inappropriate activity of hYAK3 proteins.

WO2006002829, WO2006040050 and WO2006040052 discloses certain thiazolidine compounds that are CDK1 inhibitors useful as antiproliferative agents.

WO2006047269 discloses certain thiazolone as estrogen receptors modulators having essentially 1,4-disubstituted phenyl ring which has one of the substitution as at least aralkyl or heteroaralkyl.

### SUMMARY OF THE INVENTION

The present invention provides substituted heterocyclic compounds of the general formula (I), or their pharmaceutically acceptable salts or prodrugs thereof, which by virtue of its inhibitory action on an enzyme protein tyrosine phosphatase 1B (PTP1B) are useful in the treatment of disorders mediated by PTP1B, particularly diabetes and obesity.

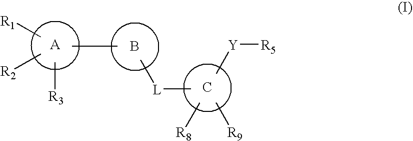

Wherein ‘A’ and ‘C’ are independently selected from aryl, heteroaryl or heterocyclyl; ‘B’ is a group selected from

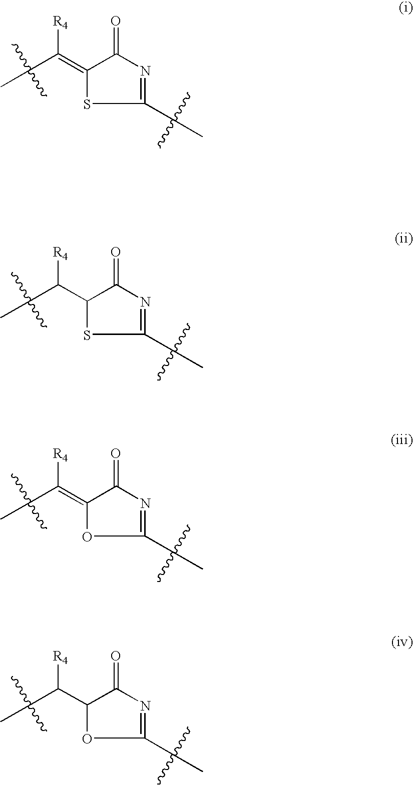

‘L’ is selected from —NH—, —NH—CH₂—, —NH—CH(CH₃)—, —NH—CH—C(O)NH—, —NH—CH₂—CH₂—, —NHNH—, —NH—CH(COOH)—CH2, —N(CH2COOH)—;

‘Y’ is selected from

‘Y’ is selected from

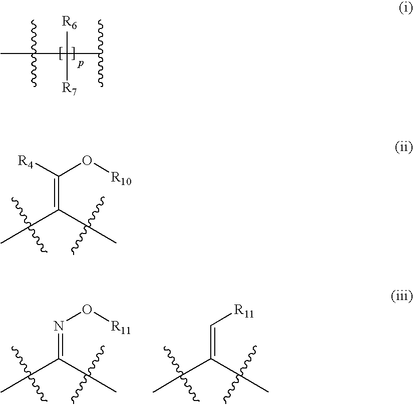

p is 1, 2 or 3;

R₁, R₂ and R₃ are independently selected from the group consisting of:
i) Hydrogen ii) —CH₂COOR₄ iii) —X(CH₂)ₙ-aryl, where n is selected from 0, 1 or 2, and X is selected from S, NH or O, iv) L-alkyl, v) —OR₄ yl) OCH₂cycloalkyl, wherein cycloalkyl is selected from cyclopropyl, cyclobutyl, cyclopentyl, cyclohexyl, cycloheptyl or cyclooctyl vii) —CH₂CONH₂, viii) —CN; ix) Halo x) alkyl xi) —SO₂aryl xii) —CF₃ xiii) —CO₂R₄ xiv) —COalkyl xv) —NH₂ xvi) —NO₂ xvii) —CH₂CONH-PhCH₂COOR₄ xviii) Tetrazolyl xix) Triazolyl xx) Pyrrolyl xxi) Benzimidazolyl xxii) Pyrazolyl xxiii) Phthalamoyl xxiv) Phenyl optionally substituted with one or more group selected from cyano, amino, carboxy, halo, nitro, alkyl, hydroxyl, —O-alkyl xxv) Biphenyl xxvi) —OCH₂CO₂R₄ xxvii) Pyridyl xxviii) Thienyl xxix) Dimethylamino xxx), —CH₂OH xxxi) —CF₂H xxxii) —SO₂NHR₄ xxxiii) NHCOalkyl xxxiv) —OCF₃

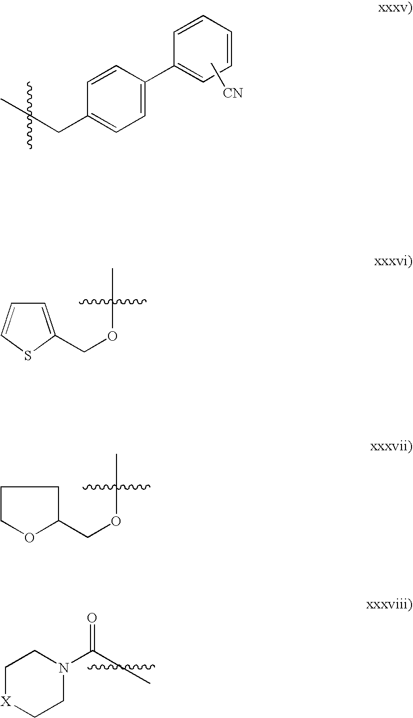

wherein x is as defined above;

R₄ is selected from hydrogen or alkyl;

R₅ is selected from the group consisting of
i) —COOR₄ wherein R₄ is as defined above,
ii) —C(O)C(O)OH,

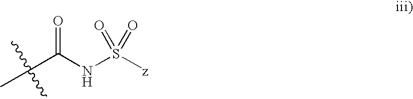

wherein z is selected from aryl or heteroarly, optionally substituted by a group independently selected from h, alkyl, Halo, nitro, —OR₄, CF₃ and CONHR₄,

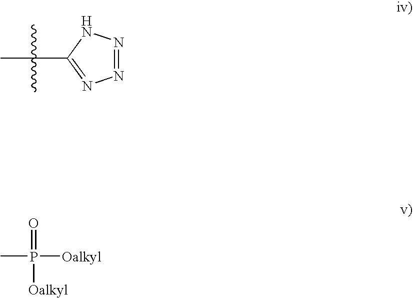

vi) —CONHOH
vii) —SO₂NHCOalkyl viii) —SO₂NHalkyl ix) —CONHSO₂alkyl x) OH
xi) —NHCOalkyl xii) —SO₂NHR₄;

R₆ is selected from the group consisting of
i) hydrogen
ii) —OR₄,
iii) alkyl,
iv) halo,
v) heterocyclyl,
vi) —S alkyl,
vii) —S-aryl,
viii) —COOR₄,
ix) Benzyl,
x) heteroaryl;

R₇ is selected from the group consisting of
i) alkyl
ii) hydrogen,
iii) halo;
   or R₆ and R₇ together may form cycloalkane ring selected from the group of cyclopentane, cyclohexane, cyclobutane;

R₈ and R₉ are independently selected from the group consisting of
i) hydrogen,
ii) halo,
iii) —OR₄,
iv) alkyl,
v) —CONHR₄
vi) —COOR₄
vii) —COalkyl;

R₁₀ is selected from the group consisting of
i) Alkyl ii) Aryl;

R₁₁ is selected from the group consisting of
i) Hydrogen ii) alkyl iii) aryl;
   and with the proviso that:
i) when R₅ is —C(O)C(O)OH, then ‘Y’ is (i), and p is null
ii) when A is phenyl then substitution at fourth position is not —X(CH₂)ₙ-aryl,

Further aspect of the invention there is provided a process for the preparation of the said compounds.

According to a further aspect of the invention there is provided a pharmaceutical composition which comprises a compound of formula (I) or their pharmaceutically acceptable salt, as defined hereinafter in association with a pharmaceutically-acceptable diluent, carrier or excipient.

Further aspect of the invention there is provided a method of inhibiting an enzyme protein tyrosine phosphatase 1B comprising administering a therapeutically effective amount of compound of general formula (I), or their pharmaceutically acceptable salt, as defined hereinafter.

Yet another aspect of the invention there is provided a method for producing a PTP1B inhibitory effect in a mammal, such as human being, in need of such treatment which comprises administering to said mammal a therapeutically effective amount of a compound of formula (I), or their pharmaceutically acceptable salt, as defined hereinafter.

According to a further feature of this aspect of the invention there is provided a method for preventing or treating disease conditions mediated by the PTP 1B enzyme, particularly diabetes mellitus and obesity in a mammal, such as human being, in need of such treatment which comprises administering to said mammal a therapeutically effective amount of a compound of formula (I), or their pharmaceutically acceptable salt, as defined hereinafter.

According to a further aspect of the present invention there is provided a compound of the formula (I) or their pharmaceutically acceptable salt, as defined hereinafter for use in a method of prophylactic or therapeutic treatment of a mammal, such as human being, and in particular for use in the treatment of diabetes mellitus and obesity.

According to another feature of the invention there is provided the use of a compound of the formula (I), or their pharmaceutically acceptable salt, as defined hereinafter in the manufacture of a medicament for use in the production of a PTP1B inhibitory effect in a mammal, such as human being.

According to another feature of the invention there is provided the use of a compound of the formula (I), or their pharmaceutically acceptable salt, as defined hereinafter in the manufacture particularly diabetes mellitus and obesity.

### BRIEF DESCRIPTION OF THE DRAWING

FIG. 1: Effect of Compound no. 28 on fasted and non-fasted blood glucose in high fat fed obese C57B116 male mice

Values are Mean±SEM

Values are calculated as % change in blood glucose from baseline blood glucose values.
a=P<0.05 vs. Vehicle

FIG. 2: Effect of 4 days treatment with Compound no. 28 on AUC blood glucose post insulin tolerance test in high fat fed obese C57BU6 male mice.

Values are Mean±SEM

a=P<0.05

### DETAILED DESCRIPTION OF THE INVENTION

In an embodiment of the present invention, there are provided compounds of formula (I),

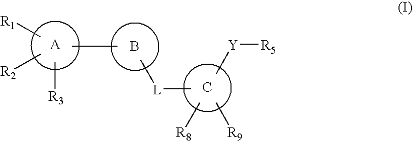

or their pharmaceutically acceptable salt and pharmaceutically acceptable compositions containing them, wherein

‘A’ is a member selected from the group consisting of:

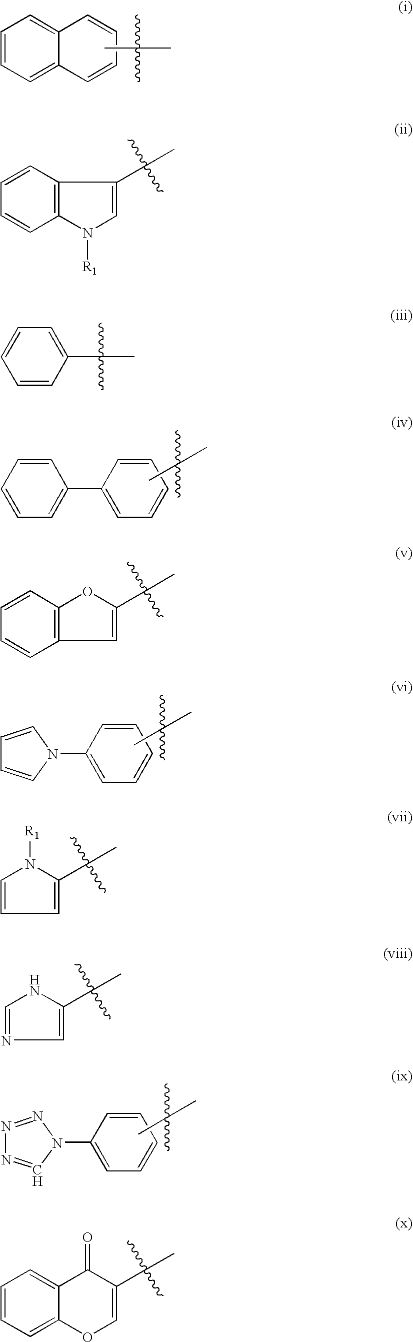

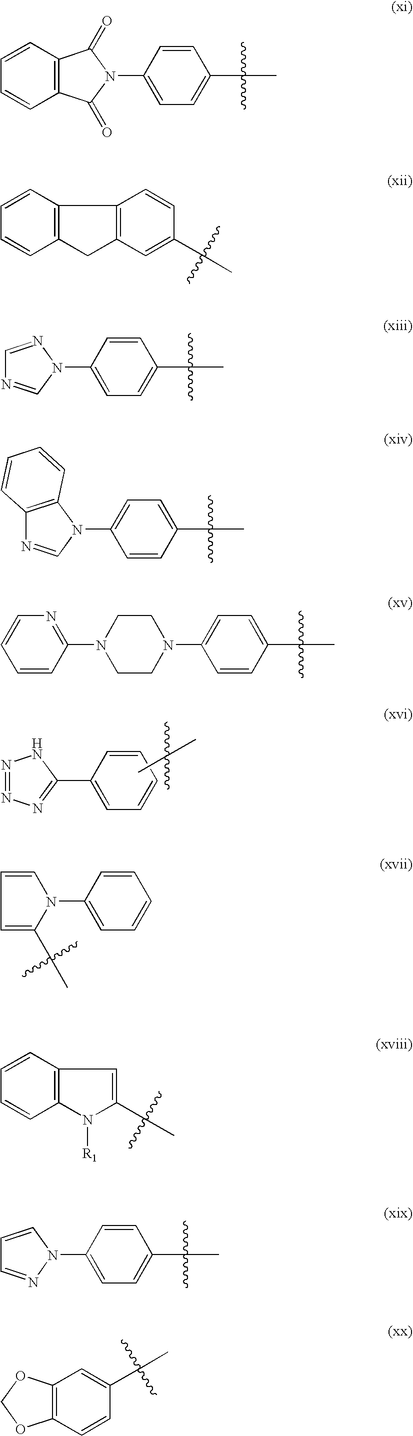

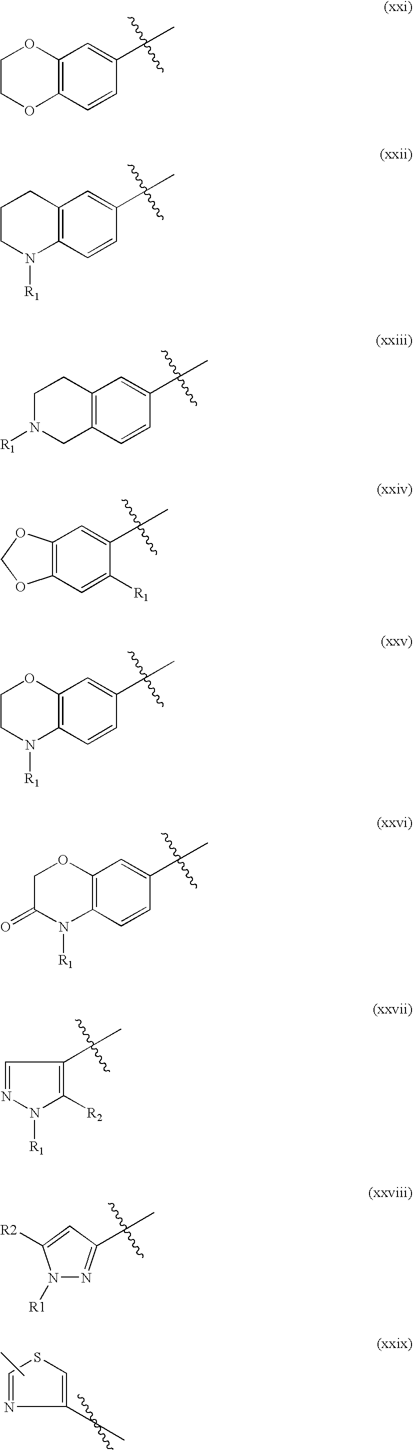

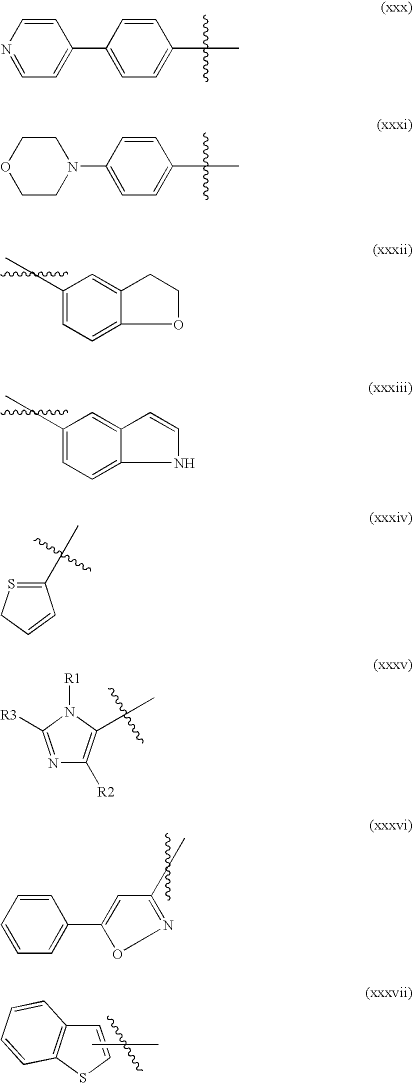

‘B’ is a group selected from

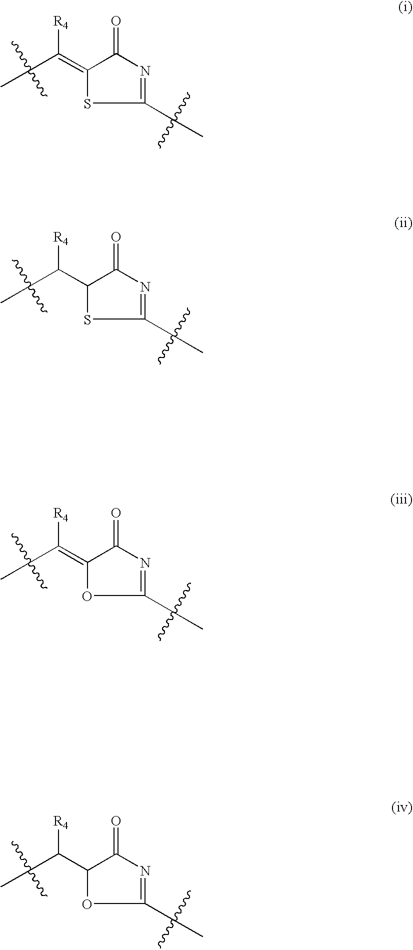

‘L’ is selected from —NH—, —NH—CH₂—, —NH—CH(CH₃)—, —NH—CH—C(O)NH—, —NH—CH₂—CH₂—, —NHNH—, —NH—CH(COOH)—CH₂, —N(CH₂COOH)—;

‘C’ is a member selected from the group consisting of:
i) Phenyl ii) Naphthyl iii) Indolyl iv) Thiazolyl v) Benzimidazolyl

‘Y’ is selected from the group consisting of

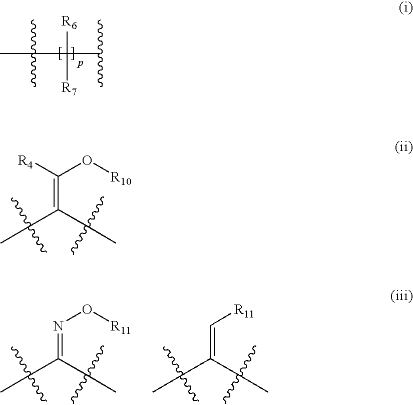

p is 1, 2 or 3;

R₁ is selected from the group consisting of:

i) Hydrogen ii) —CH₂COOR₄ iii) —X(CH₂)ₙ-aryl, where n is selected from 0, 1 or 2, and X is selected from S, NH or O, iv) —S-alkyl, v) —OR₄ vi) OCH2cycloalkyl, wherein cycloalkyl is selected from cyclopropyl, cyclobutyl, cyclopentyl, cyclohexyl, cycloheptyl or cyclooctyl vii) —CH₂CONH₂, viii) —CN, ix) Halo x) alkyl

xi) —SO₂aryl xii) —CF₃ xiii) —CO₂R xiv) —COalkyl xv) —NH₂ xvi) —NO₂ xvii) —CH₂CONH—PhCH₂COOR₄ xviii) Tetrazolyl xix) Triazolyl xx) Pyrrolyl xxi) Benzimidazolyl xxii) Pyrazolyl xxiii) Phthalamoyl xxiv) Phenyl optionally substituted with one or more group selected from cyano, amino, carboxy, halo, nitro, alkyl, hydroxyl, —O-alkyl xxv) Biphenyl xxvi) —OCH₂CO₂R₄ xxvii) Pyridyl xxviii) Thienyl xxix) Dimethylamino xxx), —CH₂OH xxxi) —CF₂H xxxii) —SO₂NHR₄ xxxiii) NHCOallyl xxxiv) —OCF₃

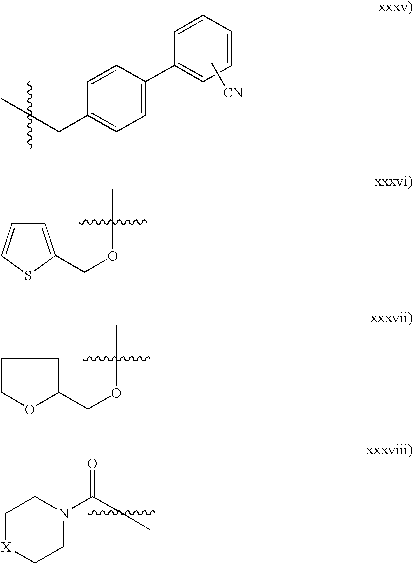

wherein x is as defined above;

R₂ and R₃ are independently selected from the group consisting of:

i) Hydrogen ii) —CH₂COOR₄ iii) —S-alkyl iv) —OR₄ v) —CH₂CONH₂ vi) —CN, vii) Halo viii) alkyl ix) —CF₃ x) —COOR₄ xi) aryl xii) —COalkyl xiii) —NH₂ xiv) —NO₂ xv) —CF₂H xvi) thienyl xvii) NHCOalkyl xviii) —OCF₃;

R₄ is selected from hydrogen or alkyl;

R₅ is selected from the group consisting of
i) —COOR₄ wherein R₄ is as defined above,
ii) —C(O)C(O)OH

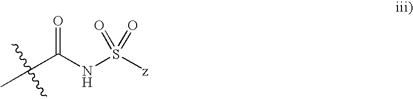

wherein z is selected from aryl or heteroaryl, optionally substituted by a group independently selected from H, alkyl, Halo, nitro, —OR₄, CF₃ and CONHR₄,

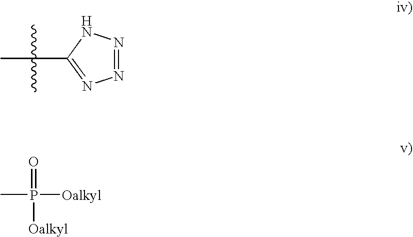

vi) —CONHOH
vii) —SO₂NHCOalkyl viii) —SO₂NHalkyl ix) —CONHSO₂alkyl x) OH xi) —NHCOalkyl xii) —SO₂NH₄;

R₆ is selected from the group consisting of
i) hydrogen,
ii) —OR₄,
iii) allyl,
iv) halo,
v) heterocyclyl,
vi) —S alkyl,
vii) —S-aryl,
viii) —COOR₄,
ix) Benzyl,
x) heteroaryl;

R₇ is selected from the group consisting of
i) alkyl,
ii) hydrogen,
iii) halo;
   or R₆ and R₇ together may form cycloalkane ring selected from the group of cyclopentane, cyclohexane, cyclobutane;

R₈ and R₉ are independently selected from the group consisting of
i) hydrogen,
ii) halo,
iii) —OR₄,
iv) allyl,
v) —CONHR₄
vi) —COOR₄,
vii) —COalkyl;

R₁₀ is selected from the group consisting of
i) Alkyl, ii) Aryl;

R₁₁ is selected from the group consisting of
i) hydrogen,
ii) alkyl,
iii) aryl;
   and with the proviso that:
i) when R₅ is —C(O)C(O)OH, then ‘Y’ is (i), and p is null
ii) when A is phenyl and is substituted by R₁ at fourth position then R₁ is not —X(CH₂)ₙ— aryl.

In another preferred embodiment of the invention, ‘A’ is a member selected from the group consisting of:

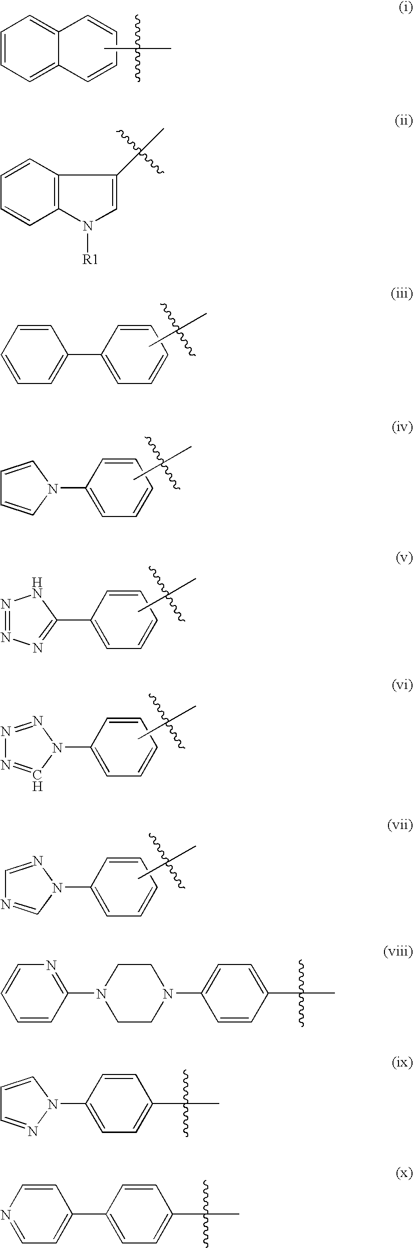

‘B’ is a group selected from

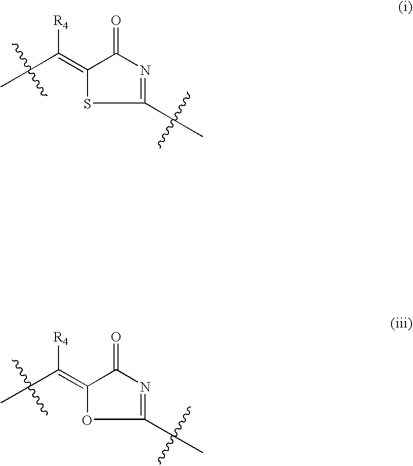

‘C’ is a member selected from the group consisting of:

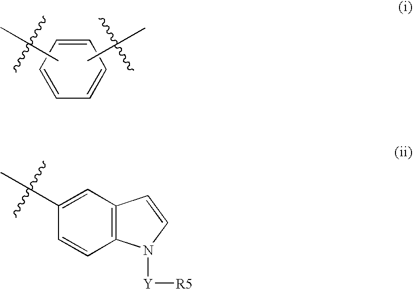

And L, Y, R₁, R₂, R₃, R₄, R₅, R₆, R₇, R₈, R₉, R₁₀, and R₁₁, are as defined hereinabove.

In a more preferred embodiment of the invention, ‘A’ is a member selected from the group consisting of:

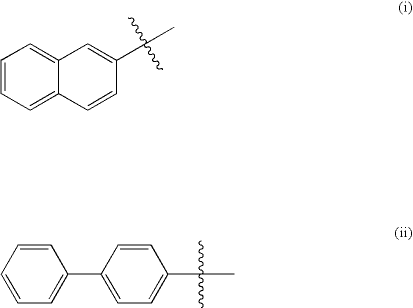

‘B’ is a group selected from

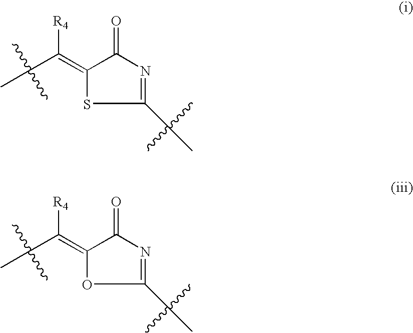

‘L’ is —NH—;

‘C’ is

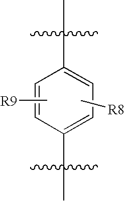

‘Y’ is

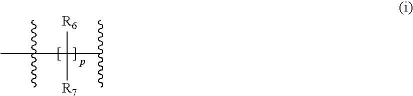

p is 1;

R₁, R₂, R₃ and R₄ are as defined hereinabove;

R₅ is selected from the group consisting of

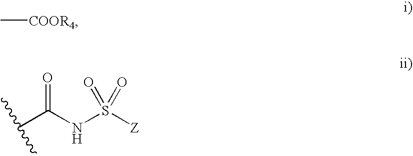

wherein z is selected from aryl or heteroaryl, optionally substituted by a group independently selected from H, alkyl, Halo, nitro, —OR₄, CF₃ and CONHR₄;

R₆ and R₇ are independently selected from hydrogen or halogen;

R₈ and R₉ are independently selected from the group consisting of
i) hydrogen,
ii) halo,
iii) —OR₄,
iv) Alkyl,
v) —CONHR₄
vi) COOR₄,
vii) —COalkyl;

A family of specific compounds of particular interest within the above formula (I) consists of compound or their pharmaceutically acceptable salts:
[4-(5-Naphthalen-2-ylmethylene-4-oxo-4,5-dihydro-thiazol-2-ylamino)-phenyl]-acetic acid. (Compound no. 1)
{4-[5-(1-Carboxymethyl-1H-indol-3-ylmethylene)-4-oxo-4,5-dihydro-thiazol-2-ylamino]-phenyl}-acetic acid. (Compound no. 2)
{4-[5-(3-Benzyloxy-naphthalen-2-ylmethylene)-4-oxo-4,5-dihydro-thiazol-2-ylamino]-phenyl}-acetic acid. (Compound no. 3)
{4-[5-(4-Methylsulfanyl-benzylidene)-4-oxo-4,5-dihydro-thiazol-2-ylamino]-phenyl}-acetic acid. (Compound no. 4)
[4-(5-Benzylidene-4-oxo-4,5-dihydro-thiazol-2-ylamino)-phenyl]-acetic acid. (Compound no. 5)
{4-[S-(4-Methoxy-naphthalen-1-ylmethylene)-4-oxo-4,5-dihydro-thiazol-2-ylamino]-phenyl}-acetic acid. (Compound no. 6)
{4-[5-(6-Methoxy-naphthalen-2-ylmethylene)-4-oxo-4,5-dihydro-thiazol-2-ylamino]-phenyl}-acetic acid. (Compound no. 7)
{4-[5-(1H-Indol-3-ylmethylene)-4-oxo-4,5-dihydro-thiazol-2-ylamino]-phenyl}-acetic acid. (Compound no. 8)
[4-(5-Biphenyl-4-ylmethylene-4-oxo-4,5-dihydro-thiazol-2-ylamino)-phenyl]-acetic acid. (Compound no. 9)
{4-[5-(3-Isopropoxy-naphthalen-2-ylmethylene)-4-oxo-4,5-dihydro-thiazol-2-ylamino]-phenyl}-acetic acid. (Compound no. 10)
{4-[5-(1-tert-Butoxycarbonylmethyl-1H-indol-3-ylmethylene)-4-oxo-4,5-dihydro-thiazol-2-ylamino]-phenyl}-acetic acid. (Compound no. 11)
[4-(4-Oxo-5-quinolin-2-ylmethylene-4,5-dihydro-thiazol-2-ylamino)-phenyl]-acetic acid. (Compound no. 12)
{4-[5-(1-Benzofuran-2-yl-ethylidene)-4-oxo-4,5-dihydro-thiazol-2-ylamino]-phenyl}-acetic acid. (Compound no. 13)
{3-[5-(3-Benzyloxy-naphthalen-2-ylmethylene)-4-oxo-4,5-dihydro-thiazol-2-ylamino]-phenyl}-acetic acid. (Compound no. 14)
{4-[5-(1-Carbamoylmethyl-1H-indol-3-ylmethylene)-4-oxo-4,5-dihydro-thiazol-2-ylamino]-phenyl}-acetic acid. (Compound no. 15)
[3-(5-Naphthalen-2-ylmethylene-4-oxo-4,5-dihydro-thiazol-2-ylamino)-phenyl]-acetic acid. (Compound no. 16)
{4-[5-(2′-Cyano-biphenyl-4-ylmethylene)-4-oxo-4,5-dihydro-thiazol-2-ylamino]-phenyl}-acetic acid. (Compound no. 17)
{4-[4-Oxo-5-(4-pyrrol-1-yl-benzylidene)-4,5-dihydro-thiazol-2-ylamino]-phenyl}-acetic acid. (Compound no. 18)
(4-{4-Oxo-5-[4-(1H-tetrazol-5-yl)-benzylidene]-4,5-dihydro-thiazol-2-ylamino}-phenyl)-acetic acid. (Compound no. 19)
(4-{5-[2-Butyl-5-chloro-3-(2′-cyano-biphenyl-4-ylmethyl)-3H-indazol-4-ylmethylene]-4-oxo-4,5-dihydro-thiazol-2-ylamino}-phenyl)-acetic acid. (Compound no. 20)
{3-[5-(3-Isopropoxy-naphthalen-2-ylmethylene)-4-oxo-4,5-dihydro-thiazol-2-ylamino]-phenyl}-acetic acid. (Compound no. 21)
(4-{2-[5-(3-Benzyloxy-naphthalen-2-ylmethylene)-4-oxo-4,5-dihydro-thiazol-2-ylamino]-acetyl amino}-phenyl)-acetic acid. (Compound no. 22)
{3-[5-(1-Carbamoylmethyl-1H-indol-3-ylmethylene)-4-oxo-4,5-dihydro-thiazol-2-ylamino]-phenyl}-acetic acid. (Compound no. 23)
[4-(S-Naphthalen-2-ylmethylene-4-oxo-4,5-dihydro-thiazol-2-ylamino)-benzyl]-phosphonic acid diethyl ester. (Compound no. 24)
4-Methyl-N-{2-[4-(5-naphthalen-2-ylmethylene-4-oxo-4,5-dihydro-thiazol-2-ylamino)-phenyl]-acetyl}-benzene sulfonamide. (Compound no. 25)
N-(2-{4-[5-(3-Benzyloxy-naphthalen-2-ylmethylene)-4-oxo-4,5-dihydro-thiazol-2-ylamino]-phenyl}-acetyl)-4-methyl-benzenesulfonamide. (Compound no. 26)
N-{2-[4-(5-Biphenyl-4-ylmethylene-4-oxo-4,5-dihydro-thiazol-2-ylamino)-phenyl]-acetyl}-4-methyl-benzenesulfonamide. (Compound no. 27)
N-{2-[4-(5-Biphenyl-4-ylmethlene-4-oxo-4,5-dihydro-thiazol-2-ylamino)-phenyl]-acetyl}-4-methyl-benzenesulfonamide sodium salt. (Compound no. 28)
(4-{4-Oxo-5-[1-(toluene-4-sulfonyl)-1H-indol-3-ylmethylene]-4,5-dihydro-thiazol-2-ylamino}-phenyl)-acetic acid. (Compound no. 29)
(3-{4-Oxo-5-[1-(toluene-4-sulfonyl)-1H-indol-3-ylmethylene]-4,5-dihydro-thiazol-2-ylamino}-phenyl)-acetic acid. (Compound no. 30)
{4-[5-(3,4-Dichloro-benzylidene)-4-oxo-4,5-dihydro-thiazol-2-ylamino]-phenyl}-acetic acid. (Compound no. 31)
{4-[5-(3-Hydroxy-naphthalen-2-ylmethylene)-4-oxo-4,5-dihydro-thiazol-2-ylamino]-phenyl}-acetic acid. (Compound no. 32)
5-(3-Benzyloxy-naphthalen-2-ylmethylene)-2-[4-(1H-tetrazol-5-ylmethyl)-phenylamino]-thiazol-4-one. (Compound no. 33)
[4-(N′-{4-Oxo-5-[4-(1H-tetrazol-5-yl)-benzylidene]-4,5-dihydro-thiazol-2-yl}-hydrazino)-phenyl]-acetic acid. (Compound no. 34)
2-[4-(5-Biphenyl-4-ylmethylene-4-oxo-4,5-dihydro-thiazol-2-ylamino)-phenyl]-N-hydroxy-acetamide. (Compound no. 35)
(4-{5-[4-(Morpholine-4-carbonyl)-benzylidene]-4-oxo-4,5-dihydro-thiazol-2-ylamino}-phenyl)-acetic acid. (Compound no. 36)
{4-[5-(3,5-Di-tert-butyl-4-hydroxy-benzylidene)-4-oxo-4,5-dihydro-thiazol-2-ylamino]-phenyl}-acetic acid. (Compound no. 37)
{4-[5-(2-Butyl-5-chloro-3H-imidazol-4-ylmethylene)-4-oxo-4,5-dihydro-thiazol-2-ylamino]-phenyl}-acetic acid. (Compound no. 38)
(4-{5-[4-(2-Fluoro-4-nitro-phenoxy)-benzylidene]-4-oxo-4,5-dihydro-thiazol-2-ylamino}-phenyl)-acetic acid. (Compound no. 39)
(4-{4-Oxo-5-[4-(piperazine-1-carbonyl)-benzylidene]-4,5-dihydro-thiazol-2-ylamino}-phenyl)-acetic acid. (Compound no. 40)
5-(6-Methoxy-naphthalen-2-ylmethylene)-2-[4-(1H-tetrazol-5-ylmethyl)-phenylamino]-thiazol-4-one. (Compound no. 41)
3-[4-(a-Naphthalen-2-ylmethylene-4-oxo-4,5-dihydro-thiazol-2-ylamino)-phenyl]-prop ionic acid. (Compound no. 42)
3-{4-[5-(1-Carboxymethyl-1H-indo-3-ylmethylene)-4-oxo-4,5-dihydro-thiazol-2-ylamino]-phenyl}-propionic acid. (Compound no. 43)
{3-[2-(4-Methylsulfamoylmethyl-phenylamino)-4-oxo-4H-thiazol-5-ylidenemethyl]-indol-1-yl}-acetic acid. (Compound no. 44)
{4-[4-Oxo-5-(4-tetrazol-1-yl-benzylidene)-4,5-dihydro-thiazol-2-ylamino]-phenyl}-acetic acid. (Compound no. 45)
3-{4-[5-(6-Methoxy-naphthalen-2-ylmethylene)-4-oxo-4,5-dihydro-thiazol-2-ylamino]-phenyl}-propionic acid. (Compound no. 46)
{5-[5-(3-Benzyloxy-naphthalen-2-ylmethylene)-4-oxo-4,5-dihydro-thiazol-2-ylamino]-indol-1-yl}-acetic acid. (Compound no. 47)
2-{4-[5-(3-Benzyloxy-naphthalen-2-ylmethylene)-4-oxo-4,5-dihydro-thiazol-2-ylamino]-phenyl}-butyric acid. (Compound no. 48)
3-{4-[5-(3-Benzyloxy-naphthalen-2-ylmethylene)-4-oxo-4,5-dihydro-thiazol-2-ylamino]-phenyl}-propionic acid. (Compound no. 49)
(4-{5-[3-(1,3-Dioxo-1,3-dihydro-isoindol-2-yl)-benzylidene]-4-oxo-4,5-dihydro-thiazol-2-ylamino}-phenyl)-acetic acid. (Compound no. 50)
{4-[4-Oxo-5-(3-pyrrol-1-yl-benzylidene)-4,5-dihydro-thiazol-2-ylamino]-phenyl}-acetic acid. (Compound no. 51)
N-Methyl-C-[4-(5-naphthalen-2-ylmethylene-4-oxo-4,5-dihydro-thriazol-2-ylamino)-phenyl]-methanesulfonamide. (Compound no. 52)
2-[4-(5-Naphthalen-2-yl-ethylene-4-oxo-4,5-dihydro-thiazol-2-ylamino)-phenyl]-propionic acid. (Compound no. 53)
2-[4-(5-Biphenyl-4-ylmethylene-4-oxo-4,5-dihydro-thiazol-2-ylamino)-phenyl]-propionic acid. (Compound no. 54)
2-[4-(5-Biphenyl-4-ylmethylene-4-oxo-4,5-dihydro-thiazol-2-ylamino)-phenyl]-succinic acid. (Compound no. 55)
{4-[c-(4-hydroxy-3,5-dimethoxy-benzylidene)-4-oxo-4,5-dihydro-thiazol-2-ylamino]-phenyl}-acetic acid. (Compound no. 56)
(4-{5-[1-(6-Methoxy-naphthalen-2-yl)-ethylidene]-4-oxo-4,5-dihydro-thiazol-2-ylamino}-phenyl)-acetic acid. (Compound no. 57)
3-{4-[5-(3,5-Di-tert-butyl)-hydroxy-benzylidene)-4-oxo-4,5-dihydro-thiazol-2-ylamino]-phenyl}-prop ionic acid. (Compound no. 58)
{4-[4-Oxo-5-(3,4,5-trimethoxy-benzylidene)-4,5-dihydro-thiazol-2-ylamino]-phenyl}-acetic acid. (Compound no. 59)
(4-{5-[1-(4-Bromo-phenyl)-1H-pyrrol-2-ylmethylene]-4-oxo-4,5-dihydro-thiazol-2-ylamino}-phenyl)-acetic acid. (Compound no. 60)
(4-{4-Oxo-5-[1-(3-pyrrol-1-yl-phenyl)-ethylidene]-4,5-dihydro-thiazol-2-ylamino}-phenyl)-acetic acid. (Compound no. 61)
{4-[4-Oxo-5-(1-phenyl-1H-pyrrol-2-ylmethylene)-4,5-dihydro-thiazol-2-ylamino]-phenyl}-acetic acid. (Compound no. 62)
C-{4-[5-(6-Methoxy-naphthalen-2-ylmethylene)-4-oxo-4,5-dihydro-thiazol-2-ylamino]-phenyl}-N-methyl-methanesulfonamide. (Compound no. 63)
C-[4-(5-Biphenyl-4-ylmethylene-4-oxo-4,5-dihydro-thiazol-2-ylamino)-phenyl]-N-ethyl-methanesulfonamide. (Compound no. 64)
C-{4-[5-(2′-Cyano-biphenyl-4-ylmethylene)-4-oxo-4,5-dihydro-thiazol-2-ylamino]-phenyl}-N-methyl-methanesulfonamide. (Compound no. 65)
[4-(5-Biphenyl-4-ylmethylene-4-oxo-4,5-dihydro-thiazol-2-ylamino)-phenyl]-bromo-acetic acid. (Compound no. 66)
2-[4-(5-Biphenyl-4-ylmethylene-4-oxo-4,5-dihydro-thiazol-2-ylamino)-benzyl]-malonic acid. (Compound no. 67)
[4-(5-Biphenyl-4-ylmethylene-4-oxo-4,5-dihydro-thiazol-2-ylamino)-phenyl]-morpholin-4-yl-acetic acid. (Compound no. 68)
N-(2-{4-[5-(6-Methoxy-naphthalen-2-ylmethylene)-4-oxo-4,5-dihydro-thiazol-2-lamino]-phenyl}-acetyl)-4-methyl-benzenesulfonamide sodium salt. (Compound no. 69)
5-Naphthalen-2-ylmethylene-2-[4-(1H-tetrazol-5-ylmethyl)-phenylamino]-thiazol-4-one. (Compound no. 70)
{4-[4-oxo-5-(4-oxo-4H-chromen-3-ylmethylene)-4,5-dihydro-thiazol-2-ylamino]-phenyl}-acetic acid. (Compound no. 71)
{4-[4-Oxo-5-(4-trifluoro methyl-benzylidene)-4,5-dihydro-thiazol-2-ylamino]-phenyl}-acetic acid. (Compound no. 72)
{4-[5-(6-Isopropoxy-naphthalen-2-ylmethylene)-4-oxo-4,5-dihydro-thiazol-2-ylamino]-phenyl}-acetic acid. (Compound no. 73)
[4-(5-Biphenyl-4-ylmethylene-4-oxo-4,5-dihydro-thiazol-2-ylamino)-phenyl]-acetic acid. (Compound no. 74)
[4-(5-Biphenyl-4-ylmethylene-4-oxo-4,5-dihydro-thiazol-2-ylamino)-phenyl]-bromo-sodium acetate. (Compound no. 75)
[4-(5-Biphenyl-4-ylmethylene-4-oxo-4,5-dihydro-thiazol-2-ylamino)-phenyl]-orpholin-4-yl-sodium acetate. (Compound no. 76)
2-[4-(5-Biphenyl-4-ylmethylene-4-oxo-4,5-dihydro-thiazol-2-ylamino)-phenyl]-propionic acid. (Compound no. 77)
[4-(5-Biphenyl-4-ylmethylene-4-oxo-4,5-dihydro-thiazol-2-ylamino)-phenyl]-sodium acetate. (Compound no. 78)
{4-[5-(3-Benzyloxy-naphthalen-2-ylmethylene)-4-oxo-4,5-dihydro-thiazol-2-ylamino]-phenyl}-sodium acetate. (Compound no. 79)
[4-(5-Naphthalen-2-ylmethylene-4-oxo-4,5-dihydro-thiazol-2-ylamino)-phenyl]-sodium acetate. (Compound no. 80)
{4-[5-(6-Methoxy-naphthalen-2-ylmethylene)-4-oxo-4,5-dihydro-thiazol-2-ylamino]-phenyl}-sodium acetate. (Compound no. 81)
{5-[5-(3-Benzyloxy-naphthalen-2-ylmethylene)-4-oxo-4,5-dihydro-thiazol-2-ylamino]-indol-1-yl}-sodium acetate. (Compound no. 82)
{4-[4-Oxo-5-(3-pyrrol-1-yl-benzylidene)-4,5-dihydro-thiazol-2-ylamino]-phenyl}-sodium acetate. (Compound no. 83)
2-{4-[L-(3-Benzyloxy-naphthalen-2-ylmethylene)-4-oxo-4,5-dihydro-thiazol-2-ylamino]-phenyl}-sodium butyrate. (Compound no. 84)
(4-{5-[3-(4-Fluoro-benzyloxy)-naphthalen-2-ylmethylene]-4-oxo-4,5-dihydro-thiazol-2-ylamino}-phenyl)-acetic acid. (Compound no. 85)
{4-[5-(9,9a-Dihydro-4aH-fluoren-2-ylmethylene)-4-oxo-4,5-dihydro-thiazol-2-ylamino]-phenyl}-acetic acid. (Compound no. 86)
{4-[4-Oxo-5-(3-[1,2,4]triazol-1-yl-benzylidene)-4,5-dihydro-thiazol-2-ylamino]-phenyl}-acetic acid. (Compound no. 87)
{4-[5-(3-Benzoimidazol-1-yl-benzylidene)-4-oxo-4,5-dihydro-thiazol-2-ylamino]-phenyl}-acetic acid. (Compound no. 88)
3-[4-(5-Naphthalen-2-yl methylene-4-oxo-4,5-dihydro-thiazol-2-ylamino)-phenyl]-sodium propionate. (Compound no. 89)
(4-{4-Oxo-5-[3-(4-pyridin-2-yl-piperazin-1-yl)-benzylidene]-4,5-dihydro-thiazol-2-ylamino}-phenyl)-acetic acid. (Compound no. 90)
{4-[4-Oxo-5-(3-pyrazol-1-yl-benzylidene)-4,5-dihydro-thiazol-2-ylamino]-phenyl}-acetic acid. (Compound no. 91)
{4-[4-Oxo-5-(3-tetrazol-1-yl-benzylidene)-4,5-dihydro-thiazol-2-ylamino]-phenyl}-sodium acetate. (Compound no. 92)
5-(3-Benzyloxy-naphthalen-2-ylmethylene)-2-[4-(5H-tetrazol-5-ylmethyl)-phenylamino]-thiazol-4-one sodium salt. (Compound no. 93)
[4-(5-Biplenyl-4-ylmethylene-4-oxo-4,5-dihydro-thiazol-2-ylamino)-phenyl]-acetic acid. (Compound no. 94)
[4-(5-Biphenyl-4-ylmethylene-4-oxo-4,5-dihydro-thiazol-2-ylamino)-phenyl]-sodium acetate. (Compound no. 95)
3-{4-[5-(3-Benzyloxy-naphthalen-2-ylmethylene)-4-oxo-4,5-dihydro-thiazol-2-lamino]-phenyl}-sodium propionate. (Compound no. 96)
[5-(5-Biphenyl-4-ylmethylene-4-oxo-4,5-dihydro-thiazol-2-ylamino)-indol-1-yl]-sodium acetate. (Compound no. 97)
5-(6-Methoxy-naphthalen-2-ylmethylene)-2-[4-(5H-tetrazol-5-ylmethyl)-phenylamino]-thiazol-4-one sodium salt. (Compound no. 98)
{5-[5-(6-Methoxy-naphthalen-2-ylmethylene)-4-oxo-4,5-dihydro-thiazol-2-ylamino]-indol-1-yl}-sodium acetate. (Compound no. 99)
[4-(5-Biphenyl-4-ylmethylene-4-oxo-4,5-dihydro-oxazol-2-ylamino)-phenyl]-acetic acid. (Compound no. 100)
[4-(5-Biphenyl-4-ylmethylene-4-oxo-4,5-dihydro-oxazol-2-ylamino)-phenyl]-sodium acetate. (Compound no. 101)
N-(2-{4-[4-Oxo-5-(4-phenyl-cyclohexa-1,5-dienylmethylene)-4,5-dihydro-thiazol-2-ylamino]-phenyl}-acetyl)-benzenesulfonamide sodium salt. (Compound no. 102)
N-(2-{4-[4-Oxo-5-(4-pyrazol-1-yl-benzylidene)-4,5-dihydro-thiazol-2-ylamino]-phenyl}-acetyl)-benzenesulfonamide. (Compound no. 103)
N-(2-{4-[5-(3-Benzyloxy-naphthalen-2-ylmethylene)-4-oxo-4,5-dihydro-thiazol-2-ylamino]-phenyl}-acetyl)-4-methyl-benzenesulfonamide sodium salt. (Compound no. 104)
N-{2-[4-(5-Biphenyl-4-ylmethylene-4-oxo-4,5-dihydro-thiazol-2-ylamino)-phenyl]-acetyl}-4-chloro-benzenesulfonamide sodium salts. (Compound no. 105)
4-Chloro-N-(2-{4-[5-(6-methoxy-naphthalen-2-ylmethylene)-4-oxo-4,5-dihydro-thiazol-2-ylamino]-phenyl}-acetyl)-benzenesulfonamide sodium salt. (Compound no. 106)
4-Chloro-N-(2-{4-[4-oxo-5-(4-pyrazol-1-yl-benzylidene)-4,5-dihydro-thiazol-2-ylamino]-phenyl}-acetyl)-benzenesulfonamide sodium salt. (Compound no. 107)
N-(2-{4-[5-(3,5-Di-tert-butyl-4-hydroxy-benzylidene)-4-oxo-4,5-dihydro-thiazol-2-ylamino]-phenyl}-acetyl)-4-methyl-benzenesulfonamide. (Compound no. 108)
[4-(4-Oxo-5-1,1′;4′,1″]terphenyl-ylmethylene-4,5-dihydro-thiazol-2-ylamino)-phenyl]-acetic acid. (Compound no. 109)
{4-[5-(6-Bromo-naphthalen-2-ylmethylene)-4-oxo-4,5-dihydro-thiazol-2-ylamino]-phenyl}-sodium acetate. (Compound no. 110)
[4-(5-Naphthalen-2-ylmethylene-4-oxo-4,5-dihydro-thiazol-2-ylamino)-phenyl]-oxo-acetic acid. (Compound No. 111)
4-Methyl-N-{2-[4-(5-naphthalen-2-ylmethylene-4-oxo-4,5-dihydro-thiazol-2-ylamino)-phenyl]-acetyl}-benzenesulfonamide sodium salt. (Compound no. 112)
(4-{5-[3-(4-Fluoro-benzyloxy)-naphthalen-2-ylmethylene-4-oxo-4,5-dihydro-thiazol-2-ylamino)-phenyl]-acetyl}-sodium acetate. (Compound no. 113)
C-{4-[5-(3-Benzyloxy-naphthalen-2-ylmethylene)-4-oxo-4,5-dihydro-thiazol-2-ylamino]-phenyl}-N-methyl-methanesulfonamide. (Compound no. 114)
{4-[5-(2-Benzyloxy-benzylidene)-4-oxo-4,5-dihydro-thiazol-2-ylamino]-phenyl}-sodium acetate. (Compound no. 115)
{4-[5-(3-Methoxy-naphthalen-2-ylmethylene)-4-oxo-4,5-dihydro-thiazol-2-ylamino]-phenyl}-sodium acetate. (Compound no. 116)
{-[5-(3-Ethoxy-naphthalen-2-ylmethylene)-4-oxo-4,5-dihydro-thiazol-2-ylamino]-phenyl}-sodium acetate. (Compound no. 117)
N-(2-{4-[5-(2-Benzyloxy-benzylidene)-4-oxo-4,5-dihydro-thiazol-2-ylamino]-phenyl}-acetyl)-4-methyl-benzenesulfonamide sodium salt. (Compound no. 118)
{4-[5-(2-Benzyloxy-4-pyrrol-1-yl-benzylidene)-4-oxo-4,5-dihydro-thiazol-2-ylamino]-phenyl}-sodium acetate. (Compound no. 119)
(4-{5-[2-(2′-Cyano-biphenyl-4-ylmethoxy)-benzylidene]-4-oxo-4,5-dihydro-thiazol-2-ylamino}-phenyl)-sodium acetate. (Compound no. 120)
[4-(5-Benzo[1,3]dioxol-5-ylmethylene-4-oxo-4,5-dihydro-thiazol-2-ylamino)-phenyl]-acetic acid. (Compound no. 121)
{4-[5-(2-Benzyloxy-4-tetrazol-1-yl-benzylidene)-4-oxo-4,5-dihydro-thiazol-2-ylamino]-phenyl}-sodium acetate. (Compound no. 122)
{4-[5-(2,3-Dihydro-benzo[1,4]dioxin-6-ylmethylene)-4-oxo-4,5-dihydro-triazol-2-ylamino]-phenyl}-acetic acid. (Compound no. 123)
{4-[4-Oxo-5-(1-phenyl-5-pyrrol-1-yl-1H-pyrazol-3-ylmethylene)-4,5-dihydro-thiazol-2-ylamino]-phenyl}-acetic acid. (Compound no. 124)
N-(2-{4-[5-(2,3-Dihydro-benzo[1,4]dioxin-6-ylmethylene)-4-oxo-4,5-dihydro-thiazol-2-ylamino]-phenyl}-acetyl)-4-methyl-benzenesulfonamide. (Compound no. 125)
N-(2-{4-[5-(2,3-Dihydro-benzo[1,4]dioxin-6-ylmethylene)-4-oxo-4,5-dihydro-thiazol-2-ylamino]-phenyl}-acetyl)-4-methyl-benzenesulfonamide. (Compound no. 126)
N-(2-{4-[5-(2-Benzyloxy-4-tetrazol-1-yl-benzylidene)-4-oxo-4,5-dihydro-thiazol-2-ylamino]-phenyl}-acetyl)-4-methyl-benzenesulfonamide sodium salt. (Compound no. 127)
[2-(5-Naphthalen-2-ylmethylene-4-oxo-4,5-dihydro-thiazol-2-ylamino)-thiazol-4-yl]-sodium acetate. (Compound no. 128)
{4-[4-Oxo-5-(3-phenethyloxy-naphthalen-2-ylmethylene)-4,5-dihydro-thiazol-2-ylamino]-phenyl}-sodium acetate. (Compound no. 129)
[2-(5-Biphenyl-4-ylmethylene-4-oxo-4,5-dihydro-thiazol-2-ylamino)-thiazol-4-yl]-acetic acid. (Compound-no. 130)
N-(2-{4-[5-(3-Benzyloxy-4-tetrazol-1-yl-benzylidene)-4-oxo-4,5-dihydro-thiazol-2-ylamino]-phenyl}-acetyl)-4-methyl-benzenesulfonamide sodium salt (Compound no. 131)
{4-[5-(5-Methyl-1-phenyl-1H-pyrazol-3-ylmethylene)-4-oxo-4,5-dihydro-thiazol-2-ylamino]-phenyl}-acetic acid. (Compound no. 132)
{2-[5-(3-Benzyloxy-naphthalen-2-ylmethylene)-4-oxo-4,5-dihydro-thiazol-2-ylamino]-thiazol-4-yl}-acetic acid. (Compound no. 133)
{2-[5-(5-Methyl-1-phenyl-1H-pyrazol-3-ylmethylene)-4-oxo-4,5-dihydro-thiazol-2-ylamino]-thiazol-4-yl}-acetic acid. (Compound no. 134)
{4-[5-(3-Cyclopentylmethoxy-naphthalen-2-ylmethylene)-4-oxo-4,5-dihydro-thiazol-2-ylamino]-phenyl}-sodium acetate. (Compound no. 135)
2-{4-[5-(3-Benzyloxy-naphthalen-2-ylmethylene)-4-oxo-4,5-dihydro-thiazol-2-ylamino]-phenyl}-3-phenyl-sodium propionate. (Compound no. 136)
{4-[5-(4-Fluoro-benzylidene)-4-oxo-4,5-dihydro-thiazol-2-ylamino]-phenyl}-acetic acid. (Compound no. 137)
{4-[4-Oxo-5-(3-propoxy-naphthalen-2-ylmethylene)-4,5-dihydro-thiazol-2-ylamino]-phenyl}-acetic acid. (Compound no. 138)
{4-[5-(1-Isopropyl-1,2,3,4-tetrahydro-quiolin-6-ylmethylene)-4-oxo-4,5-dihydro-thiazol-2-ylamino]-phenyl}-acetic acid. (Compound no. 139)
{4-[5-(3,5-Dipropoxy-naphthalen-2-ylmethylene)-4-oxo-4,5-dihydro-thiazol-2-ylamino]-phenyl}-acetic acid. (Compound no. 140)
{4-[5-(1-Methyl-1,2,3,4-tetrahydro-quinolin-6-ylmethylene)-4-oxo-4,5-dihydro-thiazol-2-ylamino]-phenyl}-acetic acid. (Compound no. 141)
(4-{4-Oxo-5-[3-(tetrahydro-furan-2-ylmethoxy)-naphthalen-2-ylmethylene]-4,5-dihydro-thiazol-2-ylamino}-phenyl)-acetic acid. (Compound no. 142)
4-[5-(6-Ethoxy-naphthalen-2-ylmethylene)-4-oxo-4,5-dihydro-thiazol-2-ylamino]-phenyl}-sodium acetate. (Compound no. 143)
{4-[5-(3,5-Diethoxy-naphthalen-2-ylmethylene)-4-oxo-4,5-dihydro-thiazol-2-ylamino]-phenyl}-sodium acetate. (Compound no. 144)
{4-[4-Oxo-5-(2-phenyl-thiazol-4-ylmethylene)-4,5-dihydro-thiazol-2-ylamino]-phenyl}-sodium acetate. (compound no. 145)
{4-[5-(1-Methoxy-naphthalen-2-ylmethylene)-4-oxo-4,5-dihydro-thiazol-2-ylamino]-phenyl}-acetic acid. (Compound no. 146)
{4-[4-Oxo-5-(4-tetrazol-1-yl-benzylidene)-4,5-dihydro-thiazol-2-ylamino]-phenyl}-sodium acetate. (Compound no. 147)
{4-[4-Oxo-5-(4-pyrrol-1-yl-benzylidene)-4,5-dihydro-thiazol-2-ylamino]-phenyl}-sodium acetate. (Compound no. 148)
{4-[5-(4-Benzyloxy-biphenyl-3-ylmethylene)-4-oxo-4,5-dihydro-thiazol-2-ylamino]-phenyl}-sodium acetate. (Compound no. 149)
{4-[5-(1-Ethoxy-naphthalen-2-ylmethylene)-4-oxo-4,5-dihydro-triazol-2-ylamino]-phenyl}-sodium acetate. (Compound no. 150)
{4-[5-(1-Benzyloxy-naphthalen-2-methylene)-4-oxo-4,5-dihydro-thiazol-2-ylamino]-phenyl}-sodium acetate. (Compound no. 151)
{4-[5-(4′-Methoxy-7-biphenyl-4-ylmethylene)-4-oxo-4,5-dihydro-thiazol-2-ylamino]-phenyl}-sodium acetate. (Compound no. 152)
[4-(5-[2,2′]Bithiophenyl-5-ylmethylene-4-oxo-4,5-dihydro-thiazol-2-ylamino)-phenyl]-sodium acetate. (Compound no. 153)
3-{4-[5-(1-Hydroxy-naphthalen-2-ylmethylene)-4-oxo-4,5-dihydro-thiazol-2-ylamino]-phenyl}-sodium propionate. (Compound no. 154)
2-{4-[5-(1-Methoxy-naphthalen-2-ylmethylene)-4-oxo-4,5-dihydro-thiazol-2-ylamino]-phenyl}-sodium butyroate. (Compound no. 155)
{5-[5-(1-Methoxy-1-naphthalen-2-ylmethylene)-4-oxo-4,5-dihydro-thiazol-2-ylamino]-indol-1-yl}-sodium acetate. (Compound no. 156)
(4-{5-[5-Bromo-2-(2′-cyano-biphenyl-4-ylmethoxy)-benzylidene]-4-oxo-4,5-dihydro-thiazol-2-ylamino}-phenyl)-sodium acetate. (Compound no. 157)
N-(2-{4-[5-(4′-Methoxy-biphenyl-4-ylmethylene)-4-oxo-4,5-dihydro-thiazol-2-ylmethyl]-phenyl}-acetyl)-4-methyl-benzenesulfonamide sodium salt. (Compound no. 158)
{5-[5-(4′-Methoxy-biphenyl-4-ylmethylene)-4-oxo-4,5-dihydro-thiazol-2-ylmethyl]-indol-1-yl}-sodium acetate. (Compound no. 159)
{4-[5-(2,3-Dihydro-benzofuran-5-ylmethylene)-4-oxo-4,5-dihydro-thiazol-2-ylamino]-phenyl}-sodium acetate. (Compound no. 160)
4-{4-[2-(4-Carboxymethyl-phenylamino)-4-oxo-4H-thiazol-5-ylidenemethyl]-phenyl}-1-methyl-pyridinium; chloride sodium salt. (Compound no. 161)
(4-{5-[3-(4-Nitro-benzyloxy)-naphthalen-2-ylmethylene]-4-oxo-4,5-dihydro-thiazol-2-ylamino}-phenyl)-acetic acid. (Compound no. 162)
{4-[5-(6-Carboxymethoxy-naphthalen-2-ylmethylene)-4-oxo-4,5-dihydro-thiazol-2-ylamino]-phenyl}-acetic acid. (Compound no. 163)
{4-[5-(3,7-Dimethoxy-naphthalen-2-ylmethylene)-4-oxo-4,5-dihydro-thiazol-2-ylamino]-phenyl}-sodium acetate. (Compound no. 164)
{4-[5-(6-Fluoro-naphthalen-2-ylmethylene)-4-oxo-4,5-dihydro-triazol-2-ylamino]-phenyl}-sodium acetate. (Compound no. 165)
{4-[5-(3,5-Dimethoxy-naphthalen-2-ylmethylene)-4-oxo-4,5-dihydro-thiazol-2-ylamino]-phenyl}-sodium acetate. (Compound no. 166)
(4-{5-[3-(4-Methyl-benzyloxy)-naphthalen-2-ylmethylene]-4-oxo-4,5-dihydro-thiazol-2-ylamino}-phenyl)-sodium acetate. (Compound no. 167)
4-Methyl-N-[2-(4-{5-[3-(4-ethyl-benzyloxy)-naphthalen-2-ylmethylene]-4-oxo-4,5-dihydro-thiazol-2-ylamino}-phenyl)-acetyl]-benzenesulfonamide sodium salt. (Compound no. 168)
(4-{-Oxo-5-[3-(thiophen-2-ylmethoxy)-naphthalen-2-ylmethylene]-4,5-dihydro-thiazol-2-ylamino}-phenyl)-sodium acetate. (Compound no. 169)
{4-[5-(H-Indol-5-ylmethylene)-4-oxo-4,5-dihydro-thiazol-2-ylamino]-phenyl}-acetic acid. (Compound no. 170)
[4-(5-Naphthalen-2-ylmethylene-4-oxo-4,5-dihydro-thiazol-2-ylamino)-phenyl]-acetic acid ethyl ester. (Compound no. 171)
3-[4-(5-Biphenyl-4-ylmethylene-4-oxo-4,5-dihydro-thiazol-2-ylamino)-phenyl]-sodium propionate. (Compound no. 172)
4-Methyl-N-[2-(4-{4-oxo-5-[4-(4-pyridin-2-yl-piperazin-1-yl)-benzylidene]-4,5-dihydro-thiazol-2-ylamino}-phenyl)-acetyl]-benzenesulfonamide sodium salt. (Compound no. 173)
{4-[5-(4′-Methyl-biphenyl-4-ylmethylene)-4-oxo-4,5-dihydro-thiazol-2-ylamino]-phenyl}-sodium acetate. (Compound no. 174)
{4-[5-(6-Isopropyl-4-oxo-4H-chromen-3-ylmethylene)-4-oxo-4,5-dihydro-triazol-2-ylamino]-phenyl}-sodium acetate. (Compound no. 175)
N-(2-{4-[5-(3-Methoxy-naphthalen-2-yl)methylene)-4-oxo-4,5-dihydro-thiazol-2-ylamino]-phenyl}-acetyl)-4-methyl-benzenesulfonamide sodium salt. (Compound no. 176)
3-{-4-[5-(3-Methoxy-naphthalen-2-ylmethylene)-4-oxo-4,5-dihydro-thiazol-2-ylamino]-phenyl}-sodium propionate. (Compound no. 177)
{4-[5-(2-Fluoro-biphenyl-4-ylmethylene)-4-oxo-4,5-dihydro-thiazol-2-ylamino]-phenyl}-sodium acetate. (Compound no. 178)
{4-[5-(3-Carboxymethoxy-naphthalen-2-ylmethylene)-4-oxo-4,5-dihydro-thiazol-2-ylamino]-phenyl}-sodium acetate. (Compound no. 179)
(4-{5-[3-(2-Methoxy-benzyloxy)-naphthalen-2-ylmethylene]-4-oxo-4,5-dihydro-thiazol-2-ylamino}-phenyl)-sodium acetate. (Compound no. 180)
(4-{5-[3-(3-Methoxy-benzyloxy)-naphthalen-2-ylmethylene]-4-oxo-4-4,5-dihydro-thiazol-2-ylamino}-phenyl)-sodium acetate. (Compound no. 181)
(4-{5-[3-(4-Chloro-benzyloxy)-naphthalen-2-ylmethylene]-4-oxo-4,5-dihydro-thiazol-2-ylamino}-phenyl)-sodium acetate. (Compound no. 182)
3-(4-{5-[3-(4-Methyl-benzyloxy)-naphthalen-2-ylmethylene]-4-oxo-4,5-dihydro-thiazol-2-ylamino}-phenyl)-sodium propionate. (Compound no. 183)
3-(4-{5-[3-(4-Chloro-benzyloxy)-naphthalen-2-ylmethylene]-4-oxo-4,5-dihydro-thiazol-2-ylamino}-phenyl)-sodium propionate. (Compound no. 184)
{4-[5-(6-Hydroxy-naphthalen-2-ylmethylene)-4-oxo-4,5-dihydro-thiazol-2-ylamino]-phenyl}-acetic acid. (Compound no. 185)
[4-(5-Biphenyl-3-ylmethylene-4-oxo-4,5-dihydro-thiazol-2-ylamino)-phenyl]-sodium acetate. (Compound no. 186)
N-(2-{4-[5-(2-Fluoro-biphenyl-4-ylmethylene)-4-oxo-4,5-dihydro-thiazol-2-ylamino]-phenyl}-acetyl)-4-methyl-benzenesulfonamide sodium salt. (Compound no. 187)
{4-[5-(3,7-Diethoxy-naphthalen-2-ylmethylene)-4-oxo-4,5-dihydro-thiazol-2-ylamino]-phenyl}sodium acetate. (Compound no. 188)
{4-[5-(4′-Fluoro-biphenyl-4-ylmethylene)-4-oxo-4,5-dihydro-thiazol-2-ylamino]-phenyl}-sodium acetate. (Compound no. 189)
{4-[—(4-Morpholin-4-yl-benzylidene)-4-oxo-4,5-dihydro-thiazol-2-ylamino]-phenyl}-sodium acetate. (Compound no. 190)
4′-[2-(4-Carboxymethyl-phenylamino)-4-oxo-4H-thiazol-5-ylidenemethyl)-]-biphenyl-4-carboxylic acid. (Compound no. 191)
{4-[4-Oxo-5-(5-phenyl-thiophen-2-ylmethylene)-4,5-dihydro-thiazol-2-ylamino]-phenyl}-sodium acetate. (Compound no. 192)
[4-(5-Benzo[b]thiophen-3-ylmethylene-4-oxo-4,5-dihydro-thiazol-2-ylamino)-phenyl]-sodium acetate. (Compound no. 193)
{4-[5-(3′-Methoxy-biphenyl-4-ylmethylene)-4-oxo-4,5-dihydro-thiazol-2-ylamino]-phenyl}-sodium acetate. (Compound no. 194)
{4-[5-(4′-Ethoxy-biphenyl-4-ylmethylene)-4-oxo-4,5-dihydro-thiazol-2-ylamino]-phenyl}-sodium acetate. (Compound no. 195)
{4-[4-Oxo-5-(4′-pyrrol-1-yl-biphenyl-4-ylmethylene)-4,5-dihydro-triazol-2-ylamino]-phenyl}-sodium acetate. (Compound no. 196)
(4-{5-[3-(2-Fluoro-benzyloxy)-naphthalen-2-ylmethylene]-4-oxo-4,5-dihydro-thiazol-2-ylamino}-phenyl)-sodium acetate. (Compound no. 197)
{4-[5-(4′-Hydroxymethyl-biphenyl-4-ylmethylene)-4-oxo-4,5-dihydro-thiazol-2-ylamino]-phenyl}-sodium acetate. (Compound no. 198)
{4-[5-(4′-Hydroxy-biphenyl-4-ylmethylene)-4-oxo-4,5-dihydro-thiazol-2-ylamino]-phenyl}-acetic acid. (Compound no. 199)
5-(5-Biphenyl-4-ylmethylene-4-oxo-4,5-dihydro-thiazol-2-ylamino)-2-carboxymethyl-benzoic acid. (Compound no. 200)
{4-[5-(4′-Carboxymethoxy-biphenyl-4-ylmethylene)-4-oxo-4,5-dihydro-thiazol-2-ylamino]-phenyl}-acetic acid. (Compound no. 201)
[4-(1-Carboxymethyl-5-naphthalen-2-yl-1H-[1,2,4]triazol-3-ylamino)-phenyl]-acetic acid. (Compound no. 202)
{4-[5-(3,8-Dimethoxy-naphthalen-2-ylmethylene)-4-oxo-4,5-dihydro-thiazol-2-ylamino]-phenyl}-acetic acid. (Compound no. 203)
4-[4-(5-Biphenyl-4-ylmethylene-4-oxo-4,5-dihydro-thiazol-2-ylamino)-phenyl]-sodium butyroate. (Compound no. 204)
3-{4-[5-(6-Fluoro-naphthalen-2-ylmethylene)-4-oxo-4,5-dihydro-thiazol-2-ylamino]-phenyl}-sodium propionate. (Compound no. 205)
[4-(5-Biphenyl-4-ylmethylene-4-oxo-4,5-dihydro-thiazol-2-ylamino)-2-fluoro-phenyl]-sodium acetate. (Compound no. 206)
{4-[5-(2′-Methoxy-biphenyl-4-ylmethylene)-4-oxo-4,5-dihydro-thiazol-2-ylamino]-phenyl}-sodium acetate, (Compound no. 207)
{4-[5-(4′-Difluoromethyl-biphenyl-4-ylmethylene)-4-oxo-4,5-dihydro-thiazol-2-ylamino]-phenyl}-sodium acetate. (Compound no. 208)
{4-[5-(3′,5′-Difluoro-biphenyl-4-ylmethylene)-4-oxo-4,5-dihydro-thiazol-2-ylamino]-phenyl}-sodium acetate. (Compound no. 209)
{4-[4-Oxo-5-(4′-sulfamoyl-biphenyl-4-ylmethylene)-4,5-dihydro-thiazol-2-ylamino]-phenyl}-acetic acid. (Compound no. 210)
{4-[5-(3-Methyl-4-pyrrol-1-yl-benzylidene)-4-oxo-4,5-dihydro-thiazol-2-ylamino]-phenyl}-sodium acetate. (Compound no. 211)
N-(2-{4-[5-(4′Hydroxy-biphenyl-4-ylmethylene)-4-oxo-4,5-dihydro-thiazol-2-ylamino]-phenyl}-acetyl)-4-methyl-benzenesulfonamide di-sodium salt (Compound no. 212)
[3-(4-Oxo-2-{4-[2-oxo-2-(toluene-4-sulfonylamino)-ethyl]-phenylamino}-4H-thiazol-5-ylidenemethyl)-naphthalen-2-yloxy]-acetic acid. (Compound no. 213)
N-(2-{4-[5-(4′-Ethoxy-biphenyl-4-ylmethylene)-4-oxo-4,5-dihydro-thiazol-2-ylamino]-phenyl}-acetyl)-4-methyl-benzenesulfonamide sodium salt. (Compound no. 214)
N-(2-{4-[5-(4′-Fluoro-biphenyl-4-ylmethylene)-4-oxo-4,5-dihydro-thiazol-2-ylamino]-phenyl}-acetyl)-4-methyl-benzenesulfonamide sodium salt. (Compound no. 215)
{4-[5-(3-Fluoro-4′-methoxy-biphenyl-4-ylmethylene)-4-oxo-4,5-dihydro-thiazol-2-ylamino]-phenyl}-sodium acetate. (Compound no. 216)
{4-[5-(3-Benzyloxy-4-tetrazol-1-yl-benzylidene)-4-oxo-4,5-dihydro-thiazol-2-ylamino]-phenyl}-sodium acetate. (Compound no. 217)
Fluoro-{2-fluoro-4-[5-(4′-methoxy-biphenyl-4-ylmethylene)-4-oxo-4,5-dihydro-thiazol-2-ylamino]-phenyl}-acetic acid. (Compound no. 218)
N-{2-[4-(5-Biphenyl-4-ylmethylene-4-oxo-4,5-dihydro-thiazol-2-ylamino)-phenyl]-acetyl}-4-trifluoromethyl-benzenesulfonamide, sodium salt. (Compound no. 219)
{4-[5-(4′-Methylsulfanyl-biphenyl-4-ylmethylene-4-oxo-4,5-dihydro-thiazol-2-ylamino]-phenyl}-sodium acetate. (Compound no. 220)
{4-[5-(2′,4′-Difluoro-biphenyl-4-ylmethylene)-4-oxo-4,5-dihydro-thiazol-2-ylamino]-phenyl}-sodium acetate. (Compound no. 221)
{2-Fluoro-4-[4-oxo-5-(4′-pyrrol-1-yl-biphenyl-4ylmethylene)-4,5-dihydro-thiazol-2-ylamino]-phenyl}-sodium acetate. (Compound no. 222)
Thiophene-2-sulfonic acid {2-[4-(5-biphenyl-4-ylmethylene-4-oxo-4,5-dihydro-thiazol-2-ylamino)-phenyl]-acetyl}-amide, sodium salt. (Compound no. 223)
N-{2-[4-(5-Biphenyl-4-ylmethylene-4-oxo-4,5-dihydro-thiazol-2-ylamino)-phenyl]-acetyl}-4-fluoro-benzenesulfonamide, sodium salt. (Compound no. 224)
N-{2-[4-(5-Biphenyl-4-ylmethylene-4-oxo-4,5-dihydro-thiazol-2-ylamino)-phenyl]-acetyl}-methanesulfonamide, sodium salt. (Compound no. 225)
N-{2-[4-(5-Biphenyl-4-ylmethylene-4-oxo-4,5-dihydro-thiazol-2-ylamino)-phenyl]-acetyl}-4-methoxy-benzenesulfonamide, sodium salt. (Compound no. 226)
{4-[4-Oxo-5-(4′-trifluoromethoxy-biphenyl-4-ylmethylene)-4,5-dihydro-thiazol-2-ylamino]-phenyl}-sodium acetate. (Compound no. 227)
{4-[5-(3-Fluoro-4′-hydroxy-biphenyl-4-ylmethylene)-4-oxo-4,5-dihydro-thiazol-2-ylamino]-phenyl}-sodium acetate. (Compound no. 228)
{4-[5-(3′-Fluoro-4′-methoxy-biphenyl-4-ylmethylene)-4-oxo-4,5-dihydro-thiazol-2-ylamino]-phenyl}-sodium acetate. (Compound no. 229)
{4-[5-(4′-Acetylamino-biphenyl-4-ylmethylene]-4-oxo-4,5-dihydro-thiazol-2-ylamino)-phenyl}-sodium acetate. (Compound no. 230)
{4-[4-Oxo-5-(4′-trifluoromethyl-biphenyl-4-ylmethylene)-4,5-dihydro-thiazol-2-ylamino]-phenyl}-sodium acetate. (Compound no. 231)
{4-[5-(4′-Cyclohexylmethoxy-biphenyl-4-ylmethylene)-4-oxo-4,5-dihydro-thiazol-2-ylamino]-phenyl}-sodium acetate. (Compound no. 232)
{4-[5-(3′-Fluoro-4′-hydroxy-biphenyl-4-ylmethylene)-4-oxo-4,5-dihydro-thiazol-2-ylamino]-phenyl}-acetic acid, disodium salt. (Compound no. 233)
{4-[5-(4′-Dimethylamino-biphenyl-4-ylmethylene)-4-oxo-4,5-dihydro-thiazol-2-ylamino]-phenyl}-sodium acetate. (Compound no. 234)
4-Methyl-N-(2-{4-[5-(4′-methylsulfanyl-biphenyl-4ylmethylene)-4-oxo-4,5-dihydro-thiazol-2-ylamino]-phenyl}-acetyl)-benzenesulfonamide sodium salt. (Compound no. 235)
{2-Fluoro-4-[5-(6-methoxy-naphthalen-2-ylmethylene)-4-oxo-4,5-dihydro-thiazol-2-ylamino]-phenyl}-sodium acetate. (Compound no. 236)
{4-[4-Oxo-5-(4-pyridin-4-yl-benzylidene-4,5-dihydro-thiazol-2-ylamino]-phenyl}-acetic acid. (Compound no. 237)
{4-[5-(3-Fluoro-4′-methylsulfanyl-biphenyl-4-ylmethylene)-4-oxo-4,5-dihydro-thiazol-2-ylamino]-phenyl}-sodium acetate. (Compound no. 238)
N-(2-{4-[5-(3-Fluoro-4′-methylsulfanyl-biphenyl-4-ylmethylene)-4-oxo-4,5-dihydro-thiazol-2-ylamino]-phenyl}-acetyl)-4-trifluoromethyl-benzenesulfonamide sodium salt. (Compound no. 239)
{4-[5-(6-Benzyloxy-naphthalen-2-ylmethylene)-4-oxo-4,5-dihydro-thiazol-2-ylamino]-phenyl}-sodium acetate. (Compound no. 240)
2-[4-(5-Biphenyl-4-ylmethylene-4-oxo-4,5-dihydro-thiazol-2-ylamino)-2-fluoro-phenyl]-3-methoxy-but-2-enoic acid ethyl ester. (Compound no. 241)
{4-[5-(4-Methyl-3-pyrrol-1-yl-benzylidene)-4-oxo-4,5-dihydro-thiazol-2-ylamino]-phenyl}-sodium acetate. (Compound no. 242)
{4-[5-(4′-Cyano-biphenyl-4-ylmethylene)-4-oxo-4,5-dihydro-thiazol-2-ylamino]-phenyl}-acetic acid. (Compound no. 243).
{4-[5-(1-Biphenyl-4-yl-ethylidene)-4-oxo-4,5-dihydro-thiazol-2-ylamino]-phenyl}-acetic acid. (Compound no. 244)
N-{2-[4-(5-Biphenyl-4-ylmethylene-4-oxo-4,5-dihydro-oxazol-2-ylamino)-phenyl]-acetyl}-4-methyl-benzenesulfonamide. (Compound no. 245)
{4-[5-(6-Isopropoxy-5-methoxy-biphenyl-3-ylmethylene)-4-oxo-4,5-dihydro-thiazol-2-ylamino]-phenyl}-acetic acid. (Compound no. 246)
{4-[5-(5-Chloro-2-methoxy-4-pyrrol-1-yl-benzylidene)-4-oxo-4,5-dihydro-thiazol-2-ylamino]-phenyl}-acetic acid. (Compound no. 247)
2-[4-(5-Biphenyl-4-ylmethylene-4-oxo-4,5-dihydro-thiazol-2-ylamino)-phenyl]-3-(4-fluoro-phenyl)-acrylic acid ethyl ester. (Compound no. 248)
N-(2-{4-[5-(1-Biphenyl-4-yl-ethylidene)-4-oxo-4,5-dihydro-thiazol-2-ylamino]-phenyl}-acetyl)-4-methyl-benzenesulfonamide. (Compound no. 249)
[4-(5-Biphenyl-4-ylmethylene-4-oxo-4,5-dihydro-thiazol-2-ylamino)-phenyl]-hydroxyimino-acetic acid ethyl ester. (Compound no. 250)
N-(2-{4-[5-(6-Isopropoxy-5-methoxy-biphenyl-3-yl-methylene)-4-oxo-4,5-dihydro-thiazol-2-ylamino]-phenyl}-acetyl)-4-methyl-benzenesulfonmide (Compound no. 251)
(4-{5-[5-(4-Methoxy-phenyl)-isoxazol-3-ylmethylene]-4-oxo-4,5-dihydro-thiazol-2-ylamino}-phenyl)-sodium acetate. (Compound no. 252)
{4-[5-(2-Fluoro-biphenyl-4-ylmethyl)-4-oxo-4,5-dihydro-oxazol-2-ylamino]-phenyl}-acetic acid. (Compound no. 253)
N-(2-{4-[5-(5-Chloro-2-methoxy-4-pyrrol-1-yl-benzylidene)-4-oxo-4,5-dihydro-thiazol-2-ylamino]-phenyl}-acetyl)-4-methyl-benzenesulfonamide. (Compound no. 254)
{4-[5-(3′,5′-Difluoro-4-methoxy-biphenyl-3-ylmethylene)-4-oxo-4,5-dihydro-thiazol-2-ylamino]-phenyl}-acetic acid. (Compound no. 255)
[4-(5-Biphenyl-4-ylmethylene-4-oxo-4,5-dihydro-thiazol-2-ylamino)-phenyl]-oxo-acetic acid. (Compound no. 256)
1-{4-[5-(2-Fluoro-biphenyl-4-ylmethylene)-4-oxo-4,5-dihydro-thiazol-2-ylamino]-phenyl}-cyclopentanecarboxylic acid sodium salt. (Compound no. 257)
N-(2-{4-[5-(6-Hydroxy-5-methoxy-biphenyl-3-ylmethylene)-4-oxo-4,5-dihydro-thiazol-2-ylamino]-phenyl}-acetyl)-4-methyl-benzenesulfonamide. (Compound no. 258)
4-Methyl-N-(2-{4-[5-(4-methyl-3-pyrrol-yl-benzylidene)-4-oxo-4,5-dihydro-thiazol-2-ylamino]-phenyl}-acetyl)-benzenesulfonamide, sodium salt. (Compound no. 259)
{4-[5-(3′,5′-Difluoro-4-methoxy-biphenyl-3-ylmethylene)-4-oxo-4,5-dihydro-thiazol-2-ylamino]-2-fluoro-phenyl}-acetic acid. (Compound no. 260)
{2-Fluoro-4-[4-oxo-5-(3,2′,4′,5′-tetrafluoro-biphenyl-4-ylmethylene)-4,5-dihydro-thiazol-2-ylamino]phenyl}-acetic acid. (Compound no. 261)
{4-[4-Oxo-(4′-propoxy-biphenyl-4-ylmethylene)-4,5-dihydro-thiazol-2-ylamino]-phenyl}-sodium acetate. (Compound no. 262)
{4-[5-(4′-Cyclopropylmethoxy-biphenyl-4-ylmethylene)-4-oxo-4,5-dihydro-thiazol-2-ylamino]-phenyl}-acetic acid. (Compound no. 263)
{4-[5-(2-Benzyloxy-3,5-diiodo-benzylidene)-4-oxo-4,5-dihydro-thiazol-2-ylamino]-phenyl}-acetic acid. (Compound no. 264)
{4-[5-(2,4′-Dimethoxy-biphenyl-3-ylmethylene)-4-oxo-4,5-dihydro-thiazol-2-ylamino]-2-fluoro-phenyl}-acetic acid. (Compound no. 265)
3-{4-[5-(6-Ethoxycarbonylmethoxy-naphthalen-2-ylmethylene)-4-oxo-4,5-dihydro-thiazol-2-ylamino]-phenyl}-propionic acid. (Compound no. 266)
Bromo-(2-fluoro-4-{5-[1-(4-nitro-phenyl)-ethylidene]-4-oxo-4,5-dihydro-thiazol-2-ylamino}-phenyl)-acetic acid. (Compound no. 267)
{4-[5-(4′-Benzylamino-biphenyl-4-ylmethylene)-4-oxo-4,5-dihydro-thiazol-2-ylamino]-2-fluoro-phenyl}-bromo-acetic acid. (Compound no. 268)
{4-[5-(3,5-Di-tert-butyl-4-hydroxy-benzylidene)-4-oxo-4,5-dihydro-oxazol-2-ylamino]-2-fluoro-phenyl}-acetic acid. (Compound no. 269)
N-(2-{4-[5-(5-Bromo-2-hydroxy-3-methoxy-benzylidene)-4-oxo-4,5-dihydro-thiazol-2-ylamino]-phenyl}-acetyl)-4-dimethoxy-benzenesulfonamide. (Compound no. 270)
2-{2-Fluoro-4-[4-oxo-5-(4-pyrrol-1-yl-benzyl)-4,5-dihydro-thiazol-2-ylamino]-phenyl}-butyric acid. (Compound no. 271)
{4-[5-(4′-Amino-biphenyl-4-ylmethylene)-4-oxo-4,5-dihydro-thiazol-2-ylamino]-phenyl}-acetic acid ethyl ester. (Compound no. 272)
Methoxy-{4-[5-(4′-nitro-biphenyl-4-ylmethylene)-4-oxo-4,5-dihydro-thiazol-2-ylamino]-phenyl}-acetic acid. (Compound no. 273)
4-Methyl-N-[2-(4-{-[4′-(4-nitro-phenylsulfanyl)-biphenyl-4-ylmethylene]-4-oxo-4,5-dihydro-thiazol-2-ylamino}-phenyl)-acetyl]-benzenesulfonamide. (Compound no. 274)
N-[2-(4-{5-[4′-(2-Fluoro-4-nitro-phenylamino)-biphenyl-4-ylmethylene]-4-oxo-4,5-dihydro-thiazol-2-ylamino}-phenyl)-acetyl]-4-methyl-benzenesulfonamide. (Compound no. 275)
[4′-(4-Oxo-2-{4-[2-oxo-2-(toluene-4-sulfonylamino)-ethyl]-phenylamino}-4H-thiazol-5-ylidenemethyl)-biphenyl-4-yl]-acetic acid butyl ester. (Compound no 276)
4-Methyl-N-[2-(4-{5-[4′-(4-nitro-phenoxy)-biphenyl-4-ylmethylene]-4-oxo-4,5-dihydro-thiazol-2-ylamino}-phenyl)-acetyl]-benzenesulfonamide. (Compound no. 277)
4-Methyl-N-(2-{4-[4-oxo-5-(4-tetrazol-1-yl-benzylidene)-4,5-dihydro-oxazol-2-ylamino]-phenyl}-acetyl)-benzenesulfonamide. (Compound no. 278)
N-(2-{4-[5-(4′-Acetyl-biphenyl-4-ylmethylene)-4-oxo-4,5-dihydro-thiazol-2-ylamino]-phenyl}-acetyl)-4-methyl-benzenesulfonamide. (Compound no. 279)
Thiophene-2-sulfonic acid (2-{4-[5-(3-ethoxy-naphthalen-2-ylmethylene)-4-oxo-4,5-dihydro-thiazol-2-ylamino]-phenyl}-acetyl)-amide. (Compound no. 280)
Bromo-{4-[5-(3,5-dipropoxy-naphthalen-2-ylmethylene)-4-oxo-4,5-dihydro-thiazol-2-ylamino]phenyl}-acetic acid. (Compound no. 281)
2-{4-[5-(4′-Amino-3-fluoro-3′-methoxycarbonyl-biphenyl-4-ylmethylene)-4-oxo-4,5-dihydro-thiazol-2-ylamino]-phenyl}-octanedioic acid. (Compound no. 282)
{2-{4-[5-(3-Isopropoxy-naphthalen-2-ylmethylene)-4-oxo-4,5-dihydro-thiazol-2-ylamino]-phenyl}-5-methyl-hexanoic acid. (Compound no. 283)
2-(4-{5-[3-(Biphenyl-4-ylmethoxy)-naphthalen-2-ylmethylene]-4-oxo-4,5-dihydro-thiazol-2-ylamino}-phenyl)-octanedioic acid. (Compound no. 284)
2-{2-Fluoro-4-[5-(5′-fluoro-4-hydroxy-2′-methoxy-biphenyl-3-ylmethylene)-4-oxo-4,5-dihydro-thiazol-2-ylamino]-phenyl}-butyric acid. (Compound no. 285)
2-(2-Fluoro-4-{5-[1-(4 nitro-phenyl)-ethylidene]-4-oxo-4,5-dihydro-oxazol-2-ylamino}-phenyl)-propionic acid. (Compound no. 286)
6-(4′-{2-[4-(Bromo-carboxy-methyl)-3-fluoro-phenylamino]-4-oxo-4H-thiazol-5-ylidenemethyl}-biphenyl-4-ylsulfanyl)-hexanoic acid. (Compound no. 287)
2-{4-[5-(4′-Dimethylamino-biphenyl-4-ylmethylene)-4-oxo-4,5-dihydro-thiazol-2-ylamino]-phenyl}-octanedioic acid. (Compound no. 288)
2-{4-[5-(5-Bromo-2-hydroxy-3-methoxy-benzylidene)-4-oxo-4,5-dihydro-thiazol-2-ylamino]-2-fluoro-phenyl}-5-methyl-hexanoic acid. (Compound no. 289)
2-{2-Fluoro-4-[4-oxo-5-(2′,4′,6′-trimethyl-biphenyl-4-ylmethylene)-4,5-dihydro-thiazol-2-ylamino]-phenyl}-butyric acid. (compound no. 290)
3-Methoxy-2-{4-[5=(4′-methoxy-biphenyl-4-ylmethylene)-4-oxo-4,5-dihydro-thiazol-2-ylamino]-phenyl}-acrylic acid. (Compound no. 291)
Bromo-{4-[5-(5-bromo-2-hydroxy-3-methoxy-benzylidene)-4-oxo-4,5-dihydro-thiazol-2-ylamino]-phenyl}-acetic acid. (Compound no. 292)
2-{2-Fluoro-4-[5-(4′-hydroxy-3′-hydroxymethyl-5′-methoxy-biphenyl-4-ylmethylene)-4-oxo-4,5-dihydro-thiazol-2-ylamino]-phenyl}-pentanoic acid. (Compound no. 293)
2-{4-[5-(5-Bromo-2-hydroxy-3-methoxy-benzylidene)-4-oxo-4,5-dihydro-thiazol-2-ylamino]-phenyl}-octanedioic acid. (Compound no. 294)
4-Amino-4′-{2-[4-(1-carboxy-ethyl)-phenylamino]-4-oxo-4H-thiazol-5-ylidene ethyl}-3′-fluoro-biphenyl-3-carboxylic acid methyl ester. (Compound no. 295)
4-Amino-4′-{2-[4-(bromo-carboxy-methyl)-3-fluoro-phenylamino]-4-oxo-4H-thiazol-5-ylidenemethyl}-3′-fluoro-biphenyl-3-carboxylic acid methyl ester. (Compound no. 296).
4-Amino-4′-{2-[4-(1-carboxy-butyl)-3-fluoro-phenylamino]-4-oxo-4H-thiazol-5-ylidenemethyl}-3′-fluoro-biphenyl-3-carboxylic acid methyl ester. (Compound no. 297)
4-Amino-4′-{2-[4-(carboxy-pyrrol-1-yl-methyl)-phenylamino]-4-oxo-4H-thiazol-5-ylidenemethyl}-3′-fluoro-biphenyl-3-carboxylic acid methyl ester. (Compound no. 298)
N-(2-{4-[5-(3-Ethoxy-naphthalen-2-ylmethylene)-4-oxo-4,5-dihydro-thiazol-2-ylamino]-phenyl}-acetyl)-4-nitro-benzenesulfonamide. (Compound no. 299)
{4-[5-(3-Benzyloxy-naphthalen-2-ylmethyl)-4-oxo-4,5-dihydro-thiazol-2-ylamino]-2-fluoro-phenyl}-acetic acid. (Compound no. 300)
N-(2-{4-[5-(4′-Dimethylamino-biphenyl-4-yl-ethylene)-4-oxo-4,5-dihydro-thiazol-2-ylamino]-phenyl}-acetyl)-4-trifluoromethyl-benzenesulfonamide. (compound no. 301)
{4-[5-(4-Acetyl-biphenyl-4-ylmethylene)-4-oxo-4,5-dihydro-thiazol-2-ylamino]-phenyl}-acetic acid. (Compound no. 302)
[4-[5-(3,5-Dipropoxy-naphthalen-2-yl-ethylene)-4-oxo-4,5-dihydro-thiazol-2-ylamino]-2-(1-hydroxy-ethyl)-phenyl]-acetic acid. (Compound no. 303)
2-{4-[5-(1,4-Diethoxy-naphthalen-2-ylmethylene)-4-oxo-4,5-dihydro-thiazol-2-ylamino]-2-fluoro-phenyl}-pentanoic acid. (Compound no. 304)
N-(2-{4-[5-(1,4-Diethoxy-naphthalen-2-ylmethylene)-4-oxo-4,5-dihydro-thiazol-2-ylamino]-phenyl}-acetyl)-3-fluoro-4-methyl-benzenesulfonamide. (Compound no. 305)
6-(4′-{2-[4-(1-Carboxy-propyl)-3-fluoro-phenylamino]-4-oxo-4H-thiazol-5-ylidenemethyl}-3′-fluoro-biphenyl-4-ylsulfanyl)-hexanoic acid. (Compound no. 306)
2-{4-[4-Oxo-5-(2′,4′,6′-trimethyl-biphenyl-4-ylmethyl)-4,5-dihydro-thiazol-2-ylamino]-phenyl}-propionic acid. (Compound no. 307)
{2-Fluoro-4-[5-(5′-fluoro-4-hydroxy-2′-methoxy-biphenyl-3-ylmethylene)-4-oxo-4,5-dihydro-thiazol-2-ylamino]-phenyl}-hydroxy-acetic acid. (Compound no. 308)
2-(4-{5-[4-(5-Carboxy-pentylsulfanyl)-3-fluoro-biphenyl-4-ylmethylene]-4-oxo-4,5-dihydro-thiazol-2-ylamino}-phenyl)-octanedioic acid. (Compound no. 309)
(4-{5-[3-(Biphenyl-4-ylmethoxy)-naphthalen-2-ylmethylene]-4-oxo-4,5-dihydro-thiazol-2-ylamino}-2-fluoro-phenyl)-bromo-acetic acid. (Compound no. 310)
4-(3-{2-[4-(1-Carboxy-butyl)-3-fluoro-phenylamino]-4-oxo-4H-thiazol-5-ylidenemethyl}-naphthalen-2-yloxymethyl)-benzoic acid ethyl ester. (Compound no. 311)
N-(2-{4-[5-(5-Bromo-2-hydroxy-3-methoxy-benzylidene)-4-oxo-4,5-dihydro-thiazol-2-ylamino]-phenyl}-acetyl)-4-nitro-benzenesulfonamide. (compound no. 312)
2-{4-[S-(4′-Dimethylamino-biphenyl-4-ylmethylene)-4-oxo-4,5-dihydro-thiazol-2-ylamino]-2-fluoro-phenyl}-6-methyl-heptanoic acid. (Compound no. 313)
Ethylsulfanyl-{4-[5-(3-isopropoxy-naphthalen-2-ylmethylene)-4-oxo-4,5-dihydro-thiazol-2-ylamino]-phenyl}-acetic acid. (Compound no. 314)
2-{4-[5-(1,4-Diethoxy-naphthalen-2-ylmethylene)-4-oxo-4,5-dihydro-thiazol-2-ylamino]-2-fluoro-phenyl}-propionic acid. (Compound no. 315)
2-{4-[5-(3,5-Dipropoxy-naphthalen-2-ylmethylene)-4-oxo-4,5-dihydro-thiazol-2-ylamino]-2-fluoro-phenyl}-butyric acid. (Compound no. 316)
6-(4′-{2-[4-(Car boxy-phenylsulfanyl-methyl)-phenylamino]-4-oxo-4H-thiazol-5-ylidenemethyl}-biphenyl-4-ylsulfanyl)-hexanoic acid. (Compound no. 317)
N-(2-{2-Fluoro-4-[4-oxo-5-(2′,4′,6′-trimethyl-biphenyl-4-ylmethylene)-4,5-dihydro-thiazol-2-ylamino]-phenyl}-acetyl)-4-propyl-benzenesulfonamide. (Compound no. 318)
N-(2-{4-[5-(2,4-Dimethoxy-benzylidene)-4-oxo-4,5-dihydro-thiazol-2-ylamino]-2-fluoro-phenyl}-acetyl)-4-methyl-benzenesulfonamide. (Compound no. 319)
N-(2-{4-[5-(4-Chloro-3-nitro-benzylidene)-4-oxo-4,5-dihydro-thiazol-2-ylamino]-phenyl}-acetyl)-4-ethyl-benzenesulfonamide. (Compound no. 320)
N-(2-{4-[5-(3,5-Di-tert-butyl-4-hydroxy-benzylidene)-4-oxo-4,5-dihydro-thiazol-2-ylamino]-phenyl}-acetyl)-4-trifluoromethyl-benzenesulfonamide. (Compounds no. 321)
N-(2-{4-[5-(5-Bromo-2-methoxy-benzylidene)-4-oxo-4,5-dihydro-thiazol-2-ylamino]-phenyl}-acetyl)-4-methyl-benzenesulfonamide. (Compound no. 322)
2-{4-[5-(3-Ethoxy-naphthalen-2-ylmethylene)-4-oxo-4,5-dihydro-thiazol-2-ylamino]-phenyl}-octanedioic acid. (Compound no. 323)
2-(4-{5-[3-(Biphenyl-4-ylmethoxy)-naphthalen-2-ylmethylene]-4-oxo-4,5-dihydro-thiazol-2-ylamino}-2-fluoro-phenyl)-butyric acid. (Compound no. 324)
N-[2-(4-{5-[3-(Biphenyl-4-ylmethoxy)-naphthalen-2-ylmethylene]-4-oxo-4,5-dihydro-thiazol-2-ylamino}-phenyl)-acetyl]-4-methoxy-benzenesulfonamide. (Compound no. 325)
4-(3-{2-[4-(Carboxy-hydroxy-methyl)-3-fluoro-phenylamino]-4-oxo-4H-thiazol-5-ylidenemethyl}-naphthalen-2-yloxymethyl)-benzoic acid ethyl ester. (Compound no. 326)
4-[3-(2-{4-[2-(4-Fluoro-benzenesulfonylamino)-2-oxo-ethyl]-phenylamino}-4-oxo-4H-thiazol-5-ylidenemethyl)-naphthalen-2-yloxymethyl]-benzoic acid ethyl ester. (Compound no. 327)
3-Fluoro-N-[2-(4-{5-[3-(4-fluoro-benzyloxy)-naphthalen-2-ylmethylene]-4-oxo-4,5-dihydro-thiazol-2-ylamino}-phenyl)-acetyl]-4-methyl-benzenesulfonamide. (Compound no. 328)
[6-(4-Oxo-2-{4-[2-oxo-2-(thiophene-2-sulfonylamino)-ethyl]-phenylamino}-4H-triazol-5-ylidenemethyl)-naphthalen-2-yloxy]-acetic acid tert-butyl ester. (Compound no. 329)
N-(2-{4-[5-(4′-Dimethylamino-biphenyl-4-ylmethylene)-4-oxo-4,5-dihydro-thiazol-2-ylamino]-phenyl}-acetyl)-methanesulfonamide. (Compound no. 330)
Bromo-{4-[5-(3,5-dipropoxy-naphthalen-2-ylmethylene)-4-oxo-4,5-dihydro-thiazol-2-ylamino]-2-fluoro-phenyl}-acetic acid. (Compound no. 331)
2-{4-[5-(6-tert-Butoxycarbonylmethoxy-naphthalen-2-ylmethylene)-4-oxo-4,5-dihydro-thiazol-2-ylamino]-2-fluoro-phenyl}-butyric acid. (Compound no. 332)
N-(2-{4-[5-(4′-Hydroxy-3′-hydroxymethyl-5′-methoxy-biphenyl-4-ylmethylene)-4-oxo-4,5-dihydro-thiazol-2-ylamino]-phenyl}-acetyl)-methanesulfonamide. (Compound no. 333)
2-{4-[5-(4′-Hydroxy-3′-hydroxymethyl-5′-methoxy-biphenyl-4-ylmethylene)-4-oxo-4,5-dihydro-thiazol-2-ylamino]-phenyl}-6-methyl-heptanoic acid. (Compound no. 334)
{2-Fluoro-4-[4-oxo-5-(3,4,5-trimethoxy-benzylidene)-4,5-dihydro-thiazol-2-ylamino]-phenyl}-acetic acid. (Compound no. 335)
4-Methyl-N-(2-{4-[4-oxo-5-(3,4,5-trimethoxy-benzylidene)-4,5-dihydro-thiazol-2-ylamino]-phenyl}-acetyl)-benzenesulfonamide. (Compound no. 336)
2-{4-[5-2′,4′,6′-trimethyl-biphenyl-4-ylmethylene)-4-oxo-4,5-dihydro-thiazol-2-ylamino]-phenyl}-octanedioic acid. (Compound no. 337)
2-{4-[5-(2,4-Dimethoxy-benzylidene)-4-oxo-4,5-dihydro-thiazol-2-ylamino]-2-fluoro-phenyl}-propionic acid. (Compound no. 338)
Bromo-{4-[5-(4-chloro-3-nitro-benzylidene)-4-oxo-4,5-dihydro-thiazol-2-ylamino]-2-fluoro-phenyl}-acetic acid. (compound no. 339)
2-{4-[5-(5′-Fluoro-4-hydroxy-2′-methoxy-biphenyl-3-ylmethylene)-4-oxo-4,5-dihydro-thiazol-2-ylamino]-phenyl}-octanedioic acid. (Compound no. 340)
Bromo-{4-[5-(3,4-dipropoxy-benzylidene)-4-oxo-4,5-dihydro-thiazol-2-ylamino]-2-fluoro-phenyl}-acetic acid. (Compound no. 341)
2-{4-[5-(3,5-Dibromo-4-hydroxy-benzylidene)-4-oxo-4,5-dihydro-thiazol-2-ylamino]-2-fluoro-phenyl}-propionic acid. (Compound no. 342)
Bromo-{2-fluoro-4-[4-oxo-5-(2′,4′,6′-methyl-biphenyl-4-ylmethylene)-4,5-dihydro-thiazol-2-ylamino]-phenyl}-acetic acid. (Compound no. 343)
4-Ethyl-N-(2-{4-[5-(2-fluoro-5-nitro-benzylidene)-4-oxo-4,5-dihydro-thiazol-2-ylamino]-phenyl}-acetyl)benzenesulfonamide. (Compound no. 344)
{4-[5-(6-tert-Butoxycarbonylmethoxy-naphthalen-2-ylmethylene)-4-oxo-4,5-dihydro-thiazol-2-ylamino]-2-fluoro-phenyl}-acetic acid. (Compound no. 345)
2-{2-Fluoro-4-[4-oxo-5-(4-trifluoromethyl-benzylidene)-4,5-dihydro-thiazol-2-ylamino]-phenyl}-pentanoic acid. (Compound no. 346)
Thiophene-2-sulfonic acid (2-{4-[5-(3,4-dipropoxy-benzylidene)-4-oxo-4,5-dihydro-thiazol-2-ylamino]-2-fluoro-phenyl}-acetyl)-amide. (Compound no. 347)
2-{4-[5-(4′-Benzylsulfanyl-biphenyl-4-ylmethylene)-4-oxo-4,5-dihydro-thiazol-2-ylamino]-2-fluoro-phenyl}-pentanoic acid. (Compound no. 348)
2-(2-Fluoro-4-{5-[4′-(2-fluoro-4-nitro-phenylamino)-biphenyl-4-ylmethylene]-4-oxo-4,5-dihydro-thiazol-2-ylamino}-phenyl)-butyric acid. (compound no. 349)
{4-[5-(5′-Fluoro-4-hydroxy-2′-methoxy-biphenyl-3-ylmethylene)-4-oxo-4,5-dihydro-thiazol-2-ylamino]-phenyl}-(4-methyl-piperazin-1-yl)-acetic acid. (Compound no. 350)
4′-[2-(4-Carboxymethyl-3-fluoro-phenylamino)-4-oxo-4H-thiazol-5-ylidenemethyl]-biphenyl-4-carboxylic acid butyl ester. (Compound no. 351)
{4-[5-(3,5-Dipropoxy-naphthalen-2-ylmethylene)-4-oxo-4,5-dihydro-oxazol-2-ylamino]-2-fluoro-phenyl}-acetic acid. (Compound no. 352)

### DEFINITIONS

As used throughout the present specification, the following terms have the meanings-indicated.

The term “PTP1B” means protein tyrosine phosphatase enzyme 1B. PTP1B as used herein refers to the enzyme in its wild-type or natural form, or can refer to any isolated or purified form. Further, the term PTP1B means either the enzyme in its full-length form or in a truncated form.

The use of the terms “a” and “an” and “the” and similar referents in the context of describing the invention (especially in the context of the following claims) are to be construed to cover both the singular and the plural, unless otherwise indicated herein or clearly contradicted by context.

In the claims which follow and in the description of the invention, except where the context requires otherwise due to express language or necessary implication, the word “comprise” or variation such as “comprises” or “comprising” is used in an inclusive sense, i.e. to specify the presence of the stated features but not to preclude the presence or addition of further features in various embodiments of the invention.

The term “compound” as used herein refers to any compound encompassed by the generic formula disclosed herein. The compound described herein may contain one or more double bonds and therefore, may exist as stereoisomer, such as double-bond isomers (i.e., geometric isomers). Accordingly, the chemical structures depicted herein encompass all possible stereoisomer of the illustrated compounds including the stereoisomerically pure form (e.g., geometrically pure) and stereoisomeric mixtures. The compounds may also exist in several tautomeric forms including the enol form, the keto form and mixtures thereof. Accordingly, the chemical structures depicted herein encompass all possible tautomeric forms of the illustrated compounds. The compounds may also possess one or more asymmetric centres or planes. Compounds of the present invention containing an asymmetrically substituted atom may be isolated in optically active or racemic forms. It is well known in the art how to prepare, optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present invention. The compound herein described may also exist in the form ester wherever possible like ester of carboxy group etc. Further, it should be understood, when partial structures of the compounds are illustrated, a dash (“-”) indicate the point of attachment of the partial structure to the rest of the molecule.

The term “alkyl” as used herein refers to a monovalent and saturated straight chain (i.e. linear) or cyclic or a branched chain containing from 1 to 8 carbon atoms, and may be unsubstituted or optionally substituted and may contain one or two double or triple bonds. Representative examples of allyl include, but are not limited to methyl, ethyl, n-propyl, isopropyl, n-butyl; isobutyl, sec-butyl, tert-butyl, n-pentyl, isopentyl, cyclohexyl, cyclopentyl and the like. The allyl groups of the present invention may be substituted with 0, 1, 2, 3 or 4 substituents independently selected from the group consisting of cyano, halo, nitro, hydroxy, carboxy, amino, alkyl.

The term “aryl” as used herein refers to an aromatic group for example, which is a 6 to 15 membered monocyclic or bicyclic or tricyclic carbon-containing ring system, which may be unsubstituted or substituted. Aryl groups having an unsaturated or partially saturated ring fused to an aromatic ring can be attached through the saturated or unsaturated part of the group. Representative examples of aryl include, but are not limited to phenyl, biphenyl, naphthyl, dihydronaphthyl, indanyl and the like. The aryl groups of the present invention may be substituted with 0, 1, 2, 3 or 4 substituents independently selected from the group consisting of cyano, halo, nitro, hydroxy, carboxy, amino, alkyl, Oalkyl, CO2alkyl.

The term “heteroaryl” as used herein refers to an aromatic group for example, which is a 5 to 10 membered monocyclic or bicyclic ring system, which has at least one heteroatom and at least one carbon atom containing ring. The term “heteroatom” as used in the specification and claims includes oxygen, sulfur and nitrogen. The heteroaryl group may be attached at any available nitrogen or carbon atom of any ring. Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like. Exemplary bicyclic heteroaryl groups include indolyl, benzothiazolyl, benzodioxolyl, benzoxazolyl, benzothienyl, quinolinyl, isoquinolinyl, benzimidazolyl, cinnolinyl, quinoxalinyl, indazolyl, pyrrolopyridyl, furopyridinyl and the like.

The term “heterocyclyl” refers to a stable, fully saturated or unsaturated nonaromatic cyclic group, for example, which is a 3 to 10 membered monocyclic or bicyclic ring system, which has at least one heteroatom in at least one carbon atom containing ring. Each ring of the heterocyclyl group containing a heteroatom may have 1, 2 or 3 heteroatoms independently selected from nitrogen, oxygen or sulfur atoms. The heterocyclyl group may be attached at any heteroatom or carbon atom of the cycle, which results in the creation of a stable structure. Exemplary monocyclic heterocyclyl groups include aziridinyl, azetidinyl, pyrrolidinyl, pyrazolinyl, imidazolinyl, imidazolidinyl, oxazolidinyl, isoxazolinyl, thiazolidinyl, isothiazolidinyl, tetrahydrofuryl, piperidinyl, piperazinyl, 2-oxopiperazinyl, 2-oxopiperidinyl, 2-oxopyrrolidinyl, 4-piperidonyl, hexahydropyrazine, hexahydropyridazine, hexahydropyrmidine, tetrahydropyranyl, morpliolinyl, thiomorpholinyl, thiomorpholinyl sulfoxide, thiomorpholinyl sulfone, isothiazolidinyl and the like. Exemplary bicyclic heterocyclyl groups include tetrahydroisoquinolinyl, benzopyranyl, indolizinyl, chromonyl, dihydroisoindolyl, dihydroquinazolinyl (such as 3,4-dihydro-4-oxo-quinazolinyl), benzothiopyranyl, dihydrobenzofuryl, dihydrobenzothienyl, dihydrobenzothiopyranyl, dihydrobenzothiopyranyl sulfone, dihydrobenzopyranyl, indolinyl, isoindolinyl, tetrahydroquinolinyl, and the like. The terms “halogen” or “halo” include fluorine, chlorine, bromine, and iodine.

All substituents (R_{L}, R₂, . . . ) described herein may be attached to the main structure at any heteroatom or carbon atom which results in creation of stable compounds.

As used herein above and throughout this application, “nitrogen” and “sulfur” include any oxidized form of nitrogen and sulfur and the quaternized form of any basic nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available staying materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syni, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”); but then is metabolically hydrolyzed to the carboxylic acid, the active entity. Additional examples include peptidyl derivatives of a compound.

The compounds of the present invention can exist as pharmaceutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salts” or “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter) or both. In yet another embodiment, “treating” or “treatment” refers to delaying the onset of the disease or disorder. As used herein, amelioration of the symptoms of a particular disorder by administration of a particular compound or pharmaceutical composition refers to any lessening, whether permanent or temporary, lasting or transient that can be attributed to or associated with administration of the composition.

“therapeutically effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject, when administered to a subject in need thereof. The therapeutic effect may be objective (i.e., measurable by some test or marker) or subjective (i.e., subject gives an indication of or feels an effect). The “therapeutically effective amount” will vary depending on the compound, mode of administration, the disease and its severity and the age, weight, etc., of the subject to be treated.

### Pharmaceutical Compositions

According to another embodiment, the invention is to provide pharmaceutical composition comprising a compound of formula (I) or their pharmaceutically acceptable salt, as defined herein in association with a pharmaceutically-acceptable diluent, carrier or excipient.

While it is possible to administer pharmaceutically effective quantity of compound of general formula (I) either individually or in combination, directly without any formulation, it is common practice to administer the compounds in the form of pharmaceutical dosage forms comprising pharmaceutically acceptable excipient(s) with an active ingredient. The compounds of general formula (I) may be administered orally, parenterally, or by inhalation in dosage unit formulations containing conventional non-toxic pharmaceutically acceptable carriers, adjuvants and vehicles. Oral administration in the form of a tablet, capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques. In addition, there is provided a pharmaceutical formulation comprising a compound of general Formula (I) and a pharmaceutically acceptable carrier. One or more compounds of general Formula (I) may be present in association with one or more non-toxic pharmaceutically acceptable carriers and/or diluents and/or adjuvants and if desired other active ingredients. The pharmaceutical compositions containing compounds of general Formula (I) may be in a form suitable for oral use, for example, as tablets, troches, lozenges; aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for examples corn starch, or alginic acid; binding agents, for example starch gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gun acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial ester derived from fatty acids and a hexitol such as polyoxyethylene sorbitol monooleate, or condensation products of ethylene oxide with partial ester derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and ester or partial ester derived from fatty acids and hexitol, anhydrides, for example sorbitan monooleate, and condensation products of the said partial ester with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water Ringer's solution and. isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general formula (I) may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Compounds of general formula (I) may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dose is appropriately decided by its form of preparation, method of administration, purpose of use and age, body weight and symptom of the patient to be treated and it is not constant.

In another embodiment of the present invention there is provided a process for the preparation of the compounds of the present invention. Representative methods for synthesizing compounds of the invention are mentioned below in the following non-limiting scheme. The starting materials can be obtained from commercial sources or can be prepared by well-established literature methods known to those of ordinary skill in the art. It is to be understood that the selection of a particular synthetic method depends on the nature of the substituents required for the desired final compound. The order of synthetic steps can be altered and known variations of the conditions and processes of the preparative procedures can be used to prepare these compounds.

Accordingly, compounds of formula (I) of the present invention can be prepared as described in the schemes below.

In one embodiment of the invention, compound of formula (I) are prepared as shown in Scheme 1.

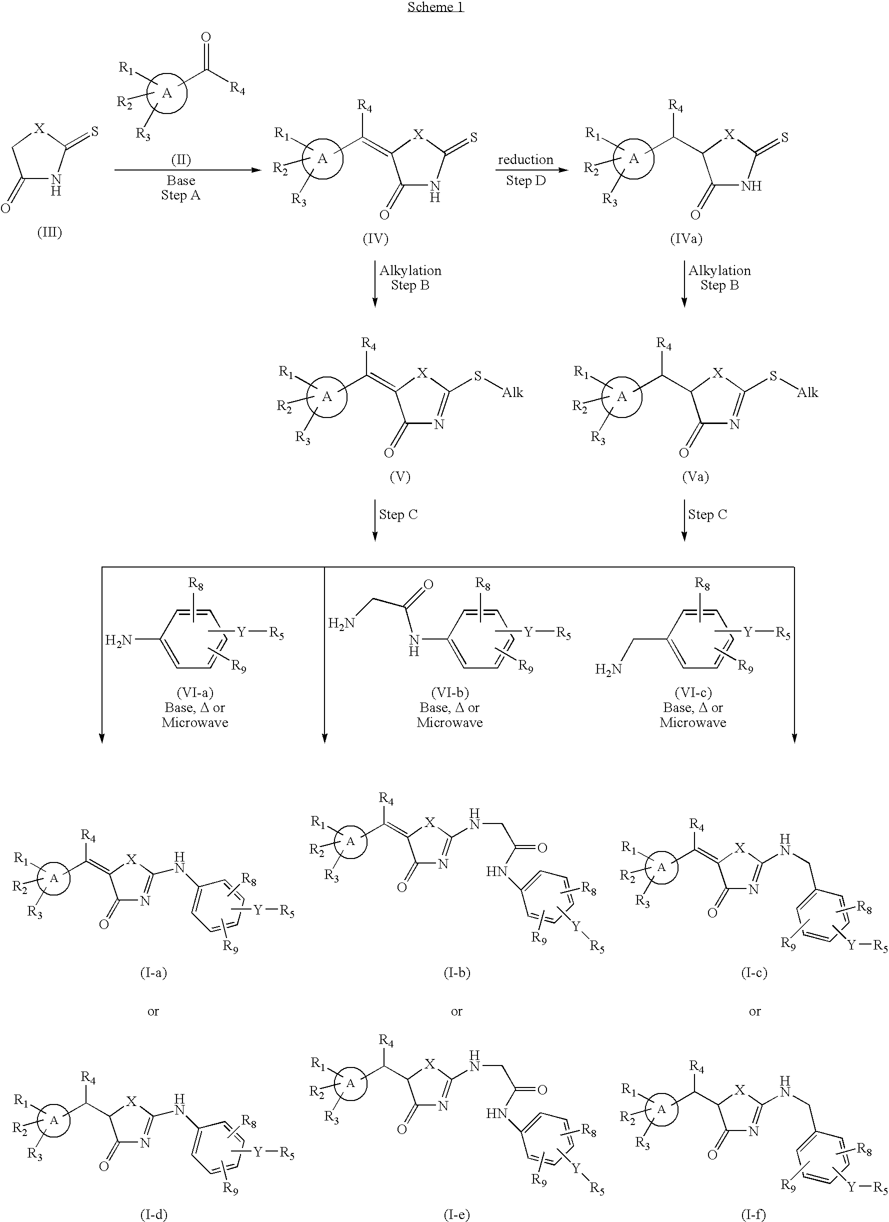

Where X=O or S, Alk=Alkyl

Y, R₁, R₂, R₃, R₄, R₅, R₈, R₉, are as defined hereinabove

As shown in Scheme 1, an appropriate aldehyde or ketone of formula (II) is reacted with a compound of formula (III) in presence of a base such as alkali metal hydroxide like sodium hydroxide and potassium hydroxide, piperidine, sodium acetate ammonium acetate etc. to provide compounds of formula (IV). The compounds of formula (IV) can be treated with a base such as Hunig's base or triethyl amine and alkyl halide preferably methyl iodide to afford compounds of formula (V). Compounds of formula (V) may be further treated with a suitable amine of formula (VI-a), (VI-b), or (VI-c) and a base such as potassium tertiary butoxide, triethylamine or hunigs base in solvents such as tertiary butanol, ethanol, n-propanol, isopropaniol etc. under reflux conditions to afford compounds of respective formula (I a-c). Furthermore, exocyclic double bond of compound of formula (IV) could be reduced to single bond of compound of formula (IVa), which could subsequently be alkylated to afford compound of formula (Va). Compounds of formula (Va) may be further treated with a suitable amine of formula (VI-a), (VI-b), or (VI-c) and a base such as potassium tertiary butoxide, triethylamine and hunigs base in solvents such as tertiary butanol, ethanol, n-propanol; isopropanol etc. under reflux conditions to afford compounds of respective formula (I d-f). The bases that can be used for the conversion of compound (V) and (Va) to compounds of formula (I a-f) can be selected from the group comprising of alkali metal alkoxides such as sodium or potassium alkoxide, alkali metal hydroxides such as sodium or potassium hydroxide, alkali metal hydrides such as sodium hydride, triethylamine, Hunig's base and the like. In an alternate embodiment, the conversion of compound (V) and (Va) to (I a-f) can also be effected by microwave irradiation. The other compounds of formula I having different L other than stated above can also be prepared by reacting formula (V) or V (a) with appropriate compound of formula (VI).

Compounds of formula (III) where X=S, (i.e. Rhodanine) used in the above scheme can be either obtained commercially or synthesized following the method as mentioned in Organic Synthesis., Collective Volume. III, p. 763.

Compounds of formula (III) where X=O, used in the above scheme can be synthesized according to the procedure described in Ushenko, N. K. and Gorizdra, T. E., Ukrain.Khim.zhur., 16, 545 (1950).

Aldehydes (II) and Amines (VI) used in scheme 1 were either commercially available or synthesized from reduction of substituted ester followed by oxidation of corresponding alcohol.

The invention is explained in detail in the examples given below which are provided by way of illustration only and therefore should not be construed to limit the scope of the invention.

### PREPARATORY EXAMPLES

### Example 1

### (4-{4-Oxo-5-[1-phenyl-methylidene]-4,5-dihydro-thiazol-2-ylamino}-phenyl)-acetic acid (1) (Compound No. 1)

### Step A: Preparation of 5-[1-Phenyl-methylidene]-2-thioxo-thiazolidin-4-one. (IV)

A mixture of benzaldehyde (II) (1 g, 9 m mol), rhodanine (1.2 g, 9 m mol) (III), and sodium acetate (2.2 g, 27 m mol) in acetic acid (10 ml) was heated under reflux temperature for 5-6 h. Subsequently the reaction mixture was cooled to room temperature, poured into water (20 ml) and the solid obtained was filtered, washed with cold methanol (5 ml) and dried to obtain 1.47 g of the title compound (IV) as a solid.

### Step B: Preparation of 2-Methyl-5-[1-phenyl-methylidene]-thiazol-4-one (V)

1.4 g (6.3 m mol) of the product (IV) from step A, was dissolved in ethanol (10 W) and stirred at 5-10° C. To the solution Hunig's base (2.2 ml, 12 m mol) was added, followed by iodomethane (0.5 ml, 7.5 m mol) and the reaction mixture stirred at room temperature for 4-5 hrs. The reaction mixture was concentrated under vacuum, water (20 ml) was added to the residue and the solid obtained was filtered and washed with ether (10 ml) to afford 1:26g of the title compound (V) as a solid.

### Step C: Preparation of (4-{4-Oxo-5-[1-phenyl-methylidene]-4,5-dihydro-thiazol-2-ylamino}-phenyl)-acetic acid (1)

A mixture of 1.2 g (5.1 m mol) of the product (V) from step B, p-amino phenyl acetic acid ethyl ester (0.9 g, 5.1 m mol) and potassium tertiary butoxide (1.2 g, 10 m mol) in tert butanol (10 ml) was heated under reflux for 6-7 hrs. The reaction mixture was concentrated under vacuo, residue was suspended in water (20 ml), and obtained solid was taken in methanol (10 ml) to which 1N NaOH solution (4.8 ml, 4.8 m mol) was added at 0-5° C. The reaction mixture was stirred at room temperature for 2 h and then concentrated under vacuo. To the residue, water (3 ml) was added and washed with diethyl ether (5 ml). Finally, the aqueous layer was acidified with 2 N HCl to afford 0.41 g of the hydrolyzed product as a solid (1).

Alternatively, Step C could be performed under microwave condition according to the following procedure.

A mixture of the product (V) from step B (1.2 g, 5.1 m mol), p-Amino phenyl acetic acid ethyl ester (0.9 g, 5.1 m mol), potassium tertiary butoxide (1.2 g, 10 m mol) in tert butanol (10 ml) was heated in a microwave synthesizer (CEM Discover®) at 100 watts for 30 min. The reaction mixture was concentrated under vacuo, residue was suspended in water (10 ml) and solid obtained was hydrolyzed as per the above-mentioned procedure to obtain 0.28 g of the title compound (1) as a solid.

¹H NMR (400 MHz, DMSO-d6) δ 3.58 (2H, s), 7.00-7.02 (1H, d), 7.28-7.33 (2H, t), 7.41-7.55 (4H, m), 7.62-7.64 (1H, d), 7.72 (1H, s), 7.95 (1H, s), 11.70 (1H, s), 12.17-12.50 (1H, bs). MS, m/z: 337 (M−1)⁻.

### Example No 2

### [4-(5-Benzyl-4-oxo-4,5-dihydro-thiazol-2-ylamino)-phenyl]-acetic acid

### Step D:—Preparation of 5-Benzyl-2-thioxo-thiazolidin-4-one (IVa)

To 1.77 g (5.9 mmol) of the product from example 124 (step A) was added a mixture of 6 ml of THF and 25 ml of water followed by 1N NaOH (3 ml) and cooled to 10° C. 1 ml of catalyst was prepared by dissolving Dimethylglyoxime (232 mg), cobaltous chloride.6H₂O (12 mg) in dimethyl formamide (5 ml), and added to the reaction mixture. The reaction mixture was stirred for 15 min, to which a solution of sodium borohydride (300 mg) and 1N NaOH (1 ml) diluted with water (3.5 ml) was added. The reaction mixture was stirred for 4 h and then acetone (2.6 ml) was added to quench any remaining sodium borohydride. After stilling for half an hour, water was added to reaction mixture and acidified with acetic acid. The solid so obtained was filtered and dissolved in ethyl acetate (20 ml). The organic layer was successively washed with water (10 ml) and brine (10 ml), dried over sodium sulphate and concentrated to yield 1 g of the title compound as a solid.

IVa was subsequently transformed into product (Example 2) following the Step B and Step C given in the example (1).

Compounds were prepared using the schemes noted above and the methods and procedures described in Example 1 or Example 2. Following table (Table-1) shows the confirmation data for some of the compounds of the present invention.

TABLE 1

| Comp. | | |
|---|---|---|
| NO | NMR Data | Mass |
| 1 | 3.59-3.60 (2H, d) 7.04 (1H, m) 7.30-7.34 (2H, t) 7.56-7.62 (3H, m) 7.72-7.73 (1H, m) | 387 (M − 1) |
| | 7.75 (1H, s) 7.88-7.93 (1H, m) 7.97-8.01 (2H, t) 8.07-8.11 (1H, m) 8.20 (1H, s) | |
| | 12.41-12.43 (1H, bs) | |
| 2 | DMSO-d6: d 3.58 (2H, s) 5.16-5.21 (2H, s) 7.03 (1H, m) 7.21-7.25 (2H, t) | 434 (M⁺ − 1) |
| | 7.27-7.31 (2H, t) 7.46-7.53 (2H, d) 7.85 (1, s) 7.89-7.93 (1H, d) 8.04 (1H, s) 12.35 (1H, s) | |
| 3 | 3.58 (2H, s) 5.35.35 (2H, d) 7.01-7.03 (1H, d) 3.58 (2H, s) 7.44-7.45 (2H, m) | 493 (M⁺ − 1) |
| | 7.48-7.59 (4H, m)7.72-7.74 (1H, d) 7.79-7.90 (2H, m) 7.99 (1H, s) 8.06-8.09 (1H, d) | |
| | 11.56 (1H, s) 12.34 (1H, s). | |
| 4 | 2.53 (3H, s) 3.58 (2H, s) 6.99-7.01 (1H, d) 7.28-7.36 (3H, m) 7.42-7.45 (2H, m) | 383 (M⁺ − 1) |
| | 7.55-7.58 (2H, m) 7.67-7.71 (1H, m) 11.36 (1H, s) 12.25 (1H, s). | |
| 5 | 3.58 (2H, s) 7.00-7.02 (1H, d) 7.28-7.33 (2H, t) 7.41-7.55 (4H, m) 7.62-7.64 (1H, d) | 337 (M − 1)⁻ |
| | 7.72 (1H, s) 7.95 (1H, s) 11.70 (1H, s) 12.17-12.50 (1H, bs). | |
| 6 | 3.57 (2H, s) 3.99 (3H, s) 7.19-7.33 (3H, m) 7.56-7.62 (2H, m) 7.66-7.76 (3H, m) | 417 (M⁺ − 1) |
| | 8.09-8.11 (1H, m) 8.16-8.28 (2H, m) 12.29-12.40 (1H, m) | |
| 7 | 3.57-3.59 (2H, m) 3.88-3.91 (3H, s) 7.01-7.03 (1H, m) 7.18-7.23 (1H, m) | 417 (M⁺ − 1) |
| | 7.26-7.33 (3H, m) 7.68-7.72 (2H, m) 7.88-7.97 (2H, m) 8.80-8.11 (1H, m). | |
| 8 | 3.59 (2H, m) 7.18-7.36 (5H, m) 8.74 (2H, m) 7.58 (1H, m) 7.74 (1H, m) 7.85-7.90 (2H, m) | 376 (M⁺ − 1) |
| | 11.99-12.26 (1H, m). | |
| 9 | 3.59-3.60 (2H, s) 7.02-7.04 (1H, m) 7.29-7.34 (2H, t) 7.37-7.41 (1H, m) 7.46-7.52 (2H, m) | 413 (M⁺ − 1) |
| | 7.61-7.63 (1H, d) 7.69-7.78 (5H, m) 7.80-7.83 (1H, d) 7.87-7.89 (1H, m) 11.64 (1H, s) | |
| | 12.39 (1H, s) | |
| 10 | 1.38-1.40 (6h, d) 3.57 (2H, s) 4.84 (1H, m) 7.03 (1H, d) 7.27-7.31 (2H, m) | 445 (M⁺ − 1) |
| | 7.40-7.49 (3H, m) 7.71 (1H, m) 7.83 (3H, m) 8.03-8.04 (1H, d) 11.36 (1H, s) 12.23 (1H, s). | |
| 11 | DMSO-d6: d 1.39-1.43 (9H, d) 3.57 (2H, s) 5.14-5.21 (2H, d) 7.00-7.01 (1H, m) | 492 (M⁺ + 1) |
| | 7.21-7.29 (4H, m) 7.40-7.48 (1H, m) 7.67-7.74 (2H, m) 7.87-7.98 (2H, m) 12.24 (1H, bs). | |
| 12 | DMSO-d6: d 3.59-3.63 (2H, d) 7.07-7.09 (1H, d) 7.32-7.34 (2H, t) 7.62-7.69 (1H, m) | 388 (M⁺ − 1) |
| | 7.77-7.79 (2H, m) 7.83-7.92 (2H, m) 7.94-8.07 (2H, m) 8.43-8.49 (1H, m) 11.04 (1H, s) | |
| | 12.34 (1H, s). | |
| 14 | 3.61-3.62 (2H, s) 5.31-5.35 (2H, d) 6.91-6.97 (1H, m) 7.09 (1H, m) 7.36-7.44 (5H, m) | 493 (M⁺ − 1) |
| | 7.50-7.59 (4H, m) 7.63-7.71 (1H, m) 7.78-7.98 (3H, m) 8.06-8.09 (1H, d) 11.36 (1H, s) | |
| | 12.41 (1H, s). | |
| 15 | DMSO-d6: d 3.59 (2H, s) 4.93-4.95 (2H, d) 7.18-7.31 (3H, m) 7.35 (1H, s) | 435 (M⁺ − 1) |
| | 7.39-7.42 (1H, d) 7.73 (1H, s) 7.80-7.81 (1H, d) 7.84-7.86 (1H, d) 7.90-7.92 (1H, d) 8.03 (1H, s) | |
| | 9.20 (1H, s) 12.36-12.37 (1H, bs). | |
| 16 | 3.43-3.87 (2H, bs) 6.97-6.99 (1H, d) 7.10-7.12 (1H, d) 7.35-7.41 (1H, q) | 387 (M⁺ − 1) |
| | 7.54-7.62 (3H, m) 7.70-7.75 (1H, m) 7.79 (1H, s) 7.89-7.93 (1H, q) 7.98-8.00 (2H, m) | |
| | 8.07-8.10 (1H, s) 11.73 (1H, s). | |
| 17 | 3.59 (2H, s) 7.04 (1H, m) 7.29-7.34 (2H, m) 7.58-7.64 (2H, m) 7.67-7.72 (4H, m) | 438 (M⁺ − 1) |
| | 7.76-7.85 (3H, m) 7.96-8.02 (1H, m) 12.43-12.46 (1H, m) | |
| 18 | 3.54-3.58 (2H, m) 6.29-6.31 (1H, m) 7.01-7.16 (1H, m) 7.29-7.38 (2H, m) | 402 (M⁺ − 1) |
| | 7.45-7.58 (2H, m) 7.66-7.72 (2H, m) 7.67-7.87 (7H, m) | |
| 19 | 3.58-3.59 (2H, d) 7.02-7.04 (1H, d) 7.29-7.33 (2H, t) 7.70-7.77 (2H, m) 7.81-7.86 (1H, m) | 405 (M⁺ − 1) |
| | 8.14-8.16 (1H, d) 8.22-8.24 (1H, d). | |
| 21 | 1.38-1.46 (6H, d) 3.60-3.64 (2H, s) 4.83-4.88 (1H, m) 6.92-6.97 (2H, d) 7.09 (1H, m) | 445 (M⁺ − 1) |
| | 7.32-7.52 (5H, m) 7.79-7.86 (2H, m) 7.94 (1H, s) 8.03-8.05 (1H, d) 11.36 (1H, s) | |
| | 12.56 (1H, s). | |
| 22 | 4.45-4.68 (2H, d) 5.33 (2H, s) 7.31 (1H, d) 7.41-7.44 (4H, m) 7.53-7.58 (4H, m) | 538 (M⁺ − 1) |
| | 7.70-7.72 (2H, d) 7.82-7.85 (1H, d) 7.90-7.95 (3H, m) 7.99-8.00 (1H, d) 10.08 (1H, s) | |
| | 10.59 (1H, s) 12.74 (1H, s). | |
| 23 | 3.60 (2H, s) 5.15-5.23 (2H, s) 6.95-6.97 (1H, m) 7.07-7.09 (1H, d) 7.21-7.26 (2H, m) | 436 (M⁺ + 1) |
| | 7.33-7.36 (1H, m) 7.46-7.47 (1H, m) 7.67 (1H, s) 7.72-7.77 (1H, m) 7.88 (2H, s) | |
| | 11.50-11.53 (1H, bs). | |
| 24 | 1.14-1.20 (6H, t) 3.22-3.28 (2H, d) 3.94-4.01 (2H, q) 7.01 (1H, m) 7.32-7.35 (2H, t) | 481 (M⁺ + 1) |
| | 7.54-7.63 (2H, m) 7.73-7.75 (1H, d) 7.79 (1H, s) 7.89 (1H, s) 7.92-7.95 (1H, t) 7.98-8.00 (1H, d) | |
| | 8.07-8.09 (1H, d) 8.20 (1H, s) 11.65 (1H, s). | |
| 25 | 1.31-1.33 (3H, d) 2.33-2.37 (2H, s) 6.90-6.98 (1H, m) 7.19-7.24 (4H, t) 7.57-7.67 (6H, m) | 540 (M⁺ − 1) |
| | 7.73-7.78 (2H, m) 7.87-7.93 (1H, m) 7.99-8.01 (2H, t) 8.06-8.14 (2H, m). | |
| 26 | 2.39 (3H, s) 3.55 (2H, s) 5.31-5.35 (2H, d) 6.97-7.20 (2H, m) 7.35-7.44 (8H, m) | 646 (M⁺ − 1) |
| | 7.49-7.60 (4H, m) 7.68-7.70 (2H, d) 7.77-7.85 (2H, m) 8.06-8.09 (2H, d) 11.63 (1H, s) | |
| | 12.28 (1H, s). | |
| 27 | DMSO-d6: δ 2.37-2.38 (3H, s) 3.56-3.57 (2H, s) 6.99 (1H, s) 7.13 (2H, t), | 566 (M⁺ − 1) |
| | 7.40-7.41 (3H, m) 7.46-7.50 (2H, m) 7.60-7.62 (1H, m) 7.69-7.71 (4H, m) 7.78-7.81 (4H, m) | |
| | 7.87-7.88 (1H, m) 11.62 (NH, s) 12.32-12.41 (1H, s). | |
| 28 | DMSO-d6: δ 2.27-2.30 (3H, s) 3.13-3.17 (2H, s) 6.68-5.70 (1H, d) 6.94-6.96 (1H, d) | 566 (M − 23)⁻ |
| | 7.02-7.04 (2H, d) 7.13-7.18 (2H, t) 7.22-7.25 (1H, d) 7.33-7.39 (1H, m) 7.43-7.49 (2H, m) | |
| | 7.51-7.53 (2H, m) 7.59-7.62 (2H, m) 7.65-7.69 (3H, t) 7.72-7.78 (1H, m). | |
| 30 | 2.87 (3H, s) 3.60 (2H, s) 7.37-7.47 (6H, m) 7.97-8.07 (8H, m). | 530 (M⁺ − 1) |
| 29 | 2.87 (3H, s) 3.60 (2H, s) 7.02-7.04 (1H, s) 7.16-7.24 (2H, m) 7.30-7.43 (4H, m) | |
| | 7.59-7.65 (1H, m) 7.65-7.76 (2H, m) 7.83-8.02 (4H, m), 11.62 (1H, s), 12.52 (1H, s). | |
| 32 | 3.58 (2H, s) 6.95 (1H, m) 7.28-7.34 (4H, m) 7.41 (1H, m) 7.66 (1H, m) 7.79-7.86 (2H, m) | 403 (M⁺ − 1) |
| | 7.95-7.97 (1H, d) 8.03-8.09 (1H, m) 10.59 (1H, s) 11.60 (1H, s) 12.38 (1H, s). | |
| 33 | 4.23 (2H, s) 5.31-5.35 (2H, d) 6.96-7.02 (1H, d) 7.20-7.39 (6H, m) 7.44-7.59 (4H, m) | 517 (M⁺ − 1) |
| | 7.70-7.72 (1H, d) 7.79-7.91 (2H, m) 7.98 (1H, s) 8.05-8.06 (1H, d) | |
| | 11.56 (1H, s) 12.50 (1H, s). | |
| 34 | 6.96-6.98 (1H, d) 7.64 (1H, s) 7.78-7.86 (4H, dd) 8.18-8.20 (2H, d) | 406 (M⁺ − 1) |
| 36 | 3.57-3.63 (10H, bs) 7.00 (1H, s) 7.28-7.33 (2H, m) 7.50-7.52 (2H, d) 7.57-7.59 (2H, d) | 450 (M⁺ − 1) |
| | 7.65-7.74 (2H, m) 12.37 (1H, bs). | |
| 41 | 3.89 (3H, s) 4.32 (2H, s) 7.05-7.07 (2H, d) 7.20-7.26 (1H, m) 7.31-7.40 (3H, m) | 441 (M⁺ − 1) |
| | 7.51-7.56 (1H, d) 7.75-7.78 (1H, d) 7.82-7.97 (3H, m) 8.03-8.12 (1H, m) 11.64 (1H, s) | |
| | 12.18 (1H, s). | |
| 42 | 2.54-2.59 (2H, m) 2.81-2.89 (2H, m) 7.02 (1H, m) 7.27-7.32 (2H, t) 7.57-7.63 (2H, m) | 401 (M⁺ − 1) |
| | 7.69-7.75 (1H, q) 7.79-7.88 (1H, d) 7.92-7.94 (1H, d) 7.97-8.02 (2H, m) 8.06-8.10 (1H, t) | |
| | 11.62 (1H, bs) 12.19-12.22 (1H, bs). | |
| 43 | 2.55-2.58 (2H, t) 2.81-2.83 (2H, t) 5.10-5.23 (2H, t) 6.98-7.00 (1H, d) 7.19-7.23 (1H, t) | 448 (M⁺ − 1) |
| | 7.26-7.27 (3H, d) 7.45-7.52 (1H, dd) 7.69 (1H, s) 7.76 (1H, s) 7.85-7.88 (2H, d) | |
| | 7.91-7.95 (1H, t) 8.04 (1H, s) 12.36 (1H, s). | |
| 44 | 2.59-2.60 (3H, 1d) 4.34 (2H, s) 5.16-5.24 (2H, m) 6.97-7.07 (2H, m) 7.21-7.27 (3H, m) | 483 (M⁺ − 1) |
| | 7.39-7.41 (2H, d) 7.47-7.49 (1H, m) 7.68 (1H, s) 7.86-7.89 (2H, m) 12.22 (1H, s) | |
| | 13.06 (1H, bs). | |
| 45 | 3.56-3.57 (2H, m) 6.97-6.99 (1H, m) 7.26-7.29 (2H, m) 7.63 (1H, m) 7.69-7.71 (1H, m) | 405 (M⁺ − 1) |
| | 7.76-7.78 (1H, d) 7.86-7.88 (1H, m) 7.99-8.02 (1H, d) 8.08-8.10 (1H, m) 10.11 (1H, m). | |
| 46 | 2.54-2.59 (2H, m) 2.80-2.86 (2H, m) 3.87-3.91 (3H, d) 6.98-7.00 (1H, m) | 433 (M⁺ + 1) |
| | 7.18-7.20 (1H, m) 7.24-7.27 (2H, m) 7.34-7.39 (1H, m) 7.64-7.70 (2H, m) 7.87-7.91 (2H, m) | |
| | 7.94-7.96 (1H, m) 8.01-8.10 (1H, m) 12.18 (1H, m). | |
| 47 | 5.02 (2H, s) 5.31-5.35 (2H, d) 7.28-7.32 (1H, m) 7.38-7.48 (7H, m) 7.50-7.59 (4H, m) | 532 (M⁺ − 1) |
| | 7.77-7.79 (1H, m) 7.84-786 (1H, d) 7.92-7.94 (1H, m) 7.99 (1H, s) 8.07-8.13 (1H, d) | |
| | 11.56 (1H, s) 12.50 (1H, s). | |
| 48 | 0.806-0.870 (3H, q) 1.63-1.70 (1H, m) 1.91-2.02 (1H, m) 3.41-3.44 (1H, t) | 521 (M⁺ − 1) |
| | 5.31-5.35 (2H, d) 7.04 (1H, m) 7.30-7.34 (4H, m) 7.42-7.45 (2H, t) 7.49-7.59 (4H, m) | |
| | 7.72-7.74 (1H, d) 7.79-7.83 (1H, m) 7.85-7.89 (1H, m) 7.92-7.98 (1H, s) 8.06-8.09 (1H, d) | |
| | 11.60 (1H, s) 12.38 (1H, s). | |
| 49 | 2.54-2.57 (2H, t) 2.81-2.84 (2H, t) 5.31-5.35 (2H, t) 6.99 (1H, m) 7.21-7.29 (2H, m) | 507 (M⁺ − 1) |
| | 7.34-7.39 (1H, m) 7.44-7.45 (3H, m) 7.52-7.56 (3H, m) 7.59 (1H, s) 7.68-7.70 (1H, d) | |
| | 7.79-7.94 (2H, m) 7.98-7.99 (1H, s) 8.06-8.09 (1H, d) 11.60 (1H, s) 12.19 (1H, s). | |
| 51 | 3.59-3.60 (2H, d) 6.25-6.32 (2H, m) 7.32-7.34 (4H, m) 7.41-7.42 (1H, m) 7.45 (1H, s) | 402 (M⁺ − 1) |
| | 7.50 (1H, m) 7.52 (1H, m) 7.63 (1H, m) 7.71-7.73 (2H, m) 7.84-7.86 (1H, m) 8.32 (1H, s) | |
| | 12.38-12.40 (1H, s) | |
| 52 | 3.43 (3H, s) 3.70-3.72 (2H, d) 6.87 (1H, s) 7.57-7.66 (3H, m) 7.71-7.75 (2H, m) | 436 (M⁺ − 1) |
| | 7.94 (2H, m) 8.06-8.11 (4H, m) 8.23-8.27 (2H, d) | |
| 53 | DMSO-d6: δ 1.36-1.40 (3H, t) 3.70-3.72 (1H, m) 7.05-7.07 (1H, d) 7.33-7.37 (2H, t) | 401 (M⁺ − 1) |
| | 7.57-7.63 (3H, m) 7.73-7.75 (2H, d) 7.80 (1H, s) 7.91-7.93 (1H, d) 7.97-8.01 (2H, t) | |
| | 8.07-8.09 (1H, s) 11.66 (NH, s) 12.36-12.47 (1H, bs). | |
| 54 | DMSO-d6: δ 1.38-1.40 (3H, s) 3.70 (1H, s) 7.05-7.06 (1H, d) 7.30-7.48 (5H, m) | 427 (M⁺ − 1) |
| | 7.61-7.63 (1H, d) 7.69-7.78 (4H, m) 7.80-7.82 (2H, d) 7.87-7.89 (1H, d) 11.63 (NH, s) | |
| | 12.39-12.50 (1H, bs). | |
| 55 | DMSO-d6: δ 2.57-2.67 (1H, M) 2.94-3.01 (1H, m) 3.12-3.24 (1H, m) 7.06 (1H, d) | 470.9 (M⁺ − 1) |
| | 7.33-7.41 (3H, m) 7.46-7.48 (2H, m) 7.61-7.63 (2H, d) 7.70-7.82 (5H, m) 7.87-7.88 (1H, d) | |
| | 11.71 (NH, s) 12.20-12.50 (2H, bs). | |
| 56 | 3.57 (2H, s) 3.74 (3H, s) 3.84 (3H, s) 6.81 (1H, s) 6.95-6.98 (1H, s) 7.04 (1H, s) | 413 (M⁺ − 1) |
| | 7.30-7.32 (2H, d) 7.66-7.72 (2H, m) 9.22 (OH, s) 12.20-12.38 (1H, bs). | |
| 58 | 1.34 (3H, s) 1.40-1.43 (15H, d) 2.53-2.55 (2H, t) 2.79-2.81 (2H, t) 7.27-7.29 (3H, m) | 479.2 (M⁺ − 1) |
| | 7.42 (1H, s) 7.67-7.70 (3H, d) 11.45 (1H, s) 12.16-12.24 (1H, bs). | |
| 60 | 3.57-3.59 (2H, s) 6.45 (2H, s) 6.71 (1H, s) 7.01 (1H, s) 7.15 (1H, s) 7.24-7.29 (2H, d) | 481 (M − 1)⁻ |
| | 7.37-7.42 (2H, t) 7.69-7.71 (1H, d) 7.80 (2H, m) | |
| 61 | 2.65-2.69 (3H, d) 3.49-3.56 (2H, d) 6.26-6.32 (2H, m) 6.85-6.87 (1H, d) 7.16-7.19 (1H, d) | 416 (M⁺ − 1) |
| | 7.27-7.29 (1H, d) 7.41-7.47 (3H, dd) 7.52-7.76 (4H, m) 11.12 (1H, s) 12.26 (1H, s). | |
| 62 | 3.57-3.60 (2H, d) 6.45 (1H, m) 6.54 (1H, m) 7.00 (1H, m) 7.18 (1H, s) 7.27-7.31 (2H, t) | 404 (M⁺ + 1) |
| | 7.34 (1H, d) 7.40-7.45 (2H, m) 7.53-7.55 (1H, m) 7.58-7.61 (2H, m) 7.66-7.71 (1H, d) | |
| | 11.51 (1H, s) 12.28-12.33 (1H, s). | |
| 63 | 2.58 (3H, m) 3.88-3.91 (3H, m) 4.34 (2H, s) 6.94-7.00 (1H, m) 7.09 (1H, m) | 468 (M⁺ + 1) |
| | 7.19-7.27 (1H, m) 7.40-7.41 (3H, m) 7.74-7.78 (1H, m) 7.88-7.95 (3H, m) 8.02-8.03 (1H, m). | |
| 65 | 2.58 (3H, s) 4.34 (2H, s) 6.94-6.99 (1H, m) 7.08 (1H, m) 7.39-7.44 (2H, m) | 510 (M + Na)⁺ |
| | 7.57-7.72 (3H, m) 7.79-7.81 (4H, m) 7.96-8.01 (1H, m). | |
| 66 | 3.60 (1H, s) 7.02-7.04 (1H, d) 7.29-7.34 (2H, t) 7.39-7.43 (1H, m) 7.45-7.47 (3H, m) | 413 (M − Br)⁻ |
| | 7.60-7.62 (1H, d) 7.69-7.77 (4H, m) 7.80-7.82 (1H, d) 7.87-7.89 (1H, d) 11.72 (1H, s) | |
| | 12.41 (1H, bs). | |
| 67 | DMSO-d6: δ 3.10-3.12 (2H, t) 3.58-3.63 (1H, q) 6.99-7.01 (1H, s) 7.27-7.31 (2H, t) | 470.9 (M⁺ − 1) |
| | 7.37-7.43 (1H, m) 7.46-7.52 (3H, m) 7.60-7.62 (1H, d) 11.67 (NH, s) | |
| | 12.66-12.76 (2H, bs). | |
| 68 | DMSO-d6: δ 2.40 (4H, s) 3.60 (4H, s) 7.08 (1H, bs) 7.34-7.52 (4H, bm) 7.61-7.66 (1h, d) | 498.1 (M⁺ − 1) |
| | 7.70-7.72 (3H, d) 7.75-7.82 (4H, m) 7.89-7.95 (2H, t) 11.77 (NH, s). | |
| 69 | DMSO-d6: δ 2.27-2.31 (3H, d) 3.12-3.17 (2H, d) 3.86-3.89 (3H, d) 6.68-6.70 (1H, d) | 570 (M⁺ + Na) |
| | 6.94-6.96 (1H, d) 7.02-7.04 (1H, d) 7.09-7.19 (3H, m) 7.23-7.27 (1H, m) 7.32 (1H, s) | |
| | 7.56-7.65 (3H, m) 7.75-7.78 (1H, m) 7.84-7.86 (2H, d) 7.94 (1H, s). | |
| 70 | 4.02 (2H, s) 7.02-7.04 (1H, m) 7.31-7.38 (2H, m) 7.58-7.61 (2H, m) 7.73-7.80 (2H, m) | 411 (M⁺ − 1) |
| | 7.89-7.94 (1H, m) 7.97-8.01 (2H, m) 8.06-8.11 (1H, d) 8.20-8.27 (1H, s) 11.68 (1H, s) | |
| | 12.49 (1H, s). | |
| 71 | 3.58 (2H, bs) 6.91-6.98 (2H, m) 7.28-7.34 (2H, m) 7.50-7.60 (2H, m) 7.68-7.76 (2H, m) | 405 (M⁺ − 1) |
| | 7.85 (1H, m) 8.77 (1H, m) | |
| 72 | 3.6 (2H, s) 7.01-7.03 (1H, d) 7.29-7.35 (2H, q) 7.72-7.74 (3H, t) 7.84-7.85 (2H, d) | 405 (M⁺ − 1) |
| | 7.91-7.93 (1H, d) 11.71 (1H, s) 12.42 (1H, bs). | |
| 73 | 1.321.36 (6H, d) 3.91 (2H, s) 4.78-4.84 (1H, d) 7.03-7.05 (1H, d) 7.15-7.22 (1H, m) | 445 (M⁺ − 1) |
| | 7.30-7.34 (3H, t) 7.52-7.55 (1H, d) 7.66-7.75 (2H, m) 7.84-7.94 (2H, m) 8.01-8.12 (1H, d) | |
| | 11.61 (1H, s) 12.37 (1H, s). | |
| 75 | DMS0-d6: δ 3.16 (1H, s) 6.68-6.70 (1H, d) 7.02-7.04 (1H, d) 7.09-711 (1H, d) | 413 (M − Na)⁻ |
| | 7.18 (1H, s) 7.21-7.24 (1H, t) 7.32-7.38 (1H, m) 7.43-7.47 (1H, m) 7.49-7.53 (1H, d) | |
| | 7.60-7.62 (1H, d) 7.65-7.70 (3H, m) 7.72-7.77 (1H, m) | |
| 76 | DMSO-d6: d 2.26 (4H, bs) 3.39 (1H, s) 3.55 (4H, s) 6.70-6.72 (1H, d) 7.18 (2H, m) | 520 (M − 1) |
| | 7.27-7.29 (1H, d) 7.32-7.36 (2H, m) 7.43-7.53 (3H, m) 7.61 (1H.,) 7.65-7.77 (4H, m). | |
| 77 | DMSO-d6: d 0.83-0.86 (3H, m) 1.67-1.69 (1H, m) 1.98 (1H, m) 3.41-3.43 (2H, m) | 441 (M⁺ − 1) |
| | 7.06 (1H, m) 7.33-7.37 (2H, t) 7.39-7.41 (1H, m) 7.44-7.48 (2H, m) 7.61-7.63 (1H, d) | |
| | 7.70-7.78 (5H, m) 7.80-7.83 (1H, d) 7.87-7.89 (1H, d) 11.63 (NH, s) 12.42-12.49 (1H, bs). | |
| 78 | 3.38 (2H, m) 6.75-6.77 (1H, d) 7.05-7.07 (1H, d) 7.12-7.14 (2H, d) 7.21 (1H, s) | 413 (M⁺ − Na) |
| | 7.27-7.29 (1H, m) 7.34-7.39 (1H, m) 7.45-7.48 (1H, t) 7.52-7.54 (1H, d) 7.61-7.63 (1H, d) | |
| | 7.65-7.69 (2H, m) 7.71-7.73 (1H, m) 7.74-7.78 (1H, m). | |
| 79 | 3.44 (2H, s) 5.29-5.32 (2H, d) 6.97-6.81 (1H, d) 7.07-7.09 (1H, d) 7.13-7.15 (1H, d) | 493 (M⁺ − Na) |
| | 7.33-7.35 (3H, m) 7.37-7.48 (4H, m) 7.53-7.56 (2H, t) 7.68 (1H, s) 7.73-7.79 (2H, m) | |
| | 7.85-7.88 (1H, t). | |
| 80 | 3.13-3.16 (2H, d) 6.69-6.71 (1H, d) 7.02-7.04 (1H, d) 7.09-7.11 (1H, d) 7.23-7.25 (1H, d) | 387 (M⁺ − Na) |
| | 7.31-7.35 (1H, d) 7.46-7.53 (2H, m) 7.61-7.70 (1H, dd) 7.83-7.87 (2H, m) | |
| | 7.88-7.96 (1H, m) 8.02 (1H, s). | |
| 82 | 4.45 (2H, s) 5.30-5.35 (2H, d) 6.31-6.37 (1H, d) 7.28-7.30 (3H, m) 7.37-7.39 (1H, d) | 532 (M⁺ − Na) |
| | 7.42-7.47 (3H, m) 7.50-7.57 (4H, m) 7.75-7.77 (1H, d) 7.83 (2H, d) 7.89-7.93 (1H, m) | |
| | 8.00-8.03 (1H, m) | |
| 83 | 3.10-3.12 (2H, d) 6.25-6.30 (2H, d) 6.66-6.68 (2H, d) 7.00 (1H, m) 7.02-7.09 (2H, m) | 402 (M⁺ − Na) |
| | 7.22 (2H, m) 7.31-7.33 (2H, m) 7.36-7.43 (2H, m) 7.62 (1H, m). | |
| 84 | 0.80-0.85 (3H, q) 1.53-1.59 (1H, m) 1.88-1.94 (1H, m) 3.14 (1H, m) 5.29-5.31 (2H, d) | 521 (M⁺ − Na) |
| | 6.73-6.75 (1H, d) 7.08-7.10 (1H, d) 7.14-7.16 (1H, d) 7.24-7.28 (1H, m) 7.35-7.40 (2H, m) | |
| | 7.43-7.46 (4H, m) 7.53-7.56 (2H, m) 7.63 (1H, s) 7.69-7.78 (1H, m) 7.84-7.87 (1H, d) | |
| | 8.02 (1H, s) | |
| 85 | 3.58 (2H, s) 5.29-5.33 (1H, d) 7.01 (1H, m) 7.27-7.33 (4H, m) 7.44 (1H, m) | 511 (M⁺ − 1) |
| | 7.51-7.60 (4H, m) 7.72-7.74 (1H, d) 7.79-7.91 (2H, m) 7.95 (1H, s) 8.06 (1H, s) 11.63 (1H, s) | |
| | 12.38 (1H, s). | |
| 86 | 3.59-3.61 (2H, d) 3.98-4.04 (2H, d) 7.03-7.05 (1H, d) 7.30-7.34 (2H, t) 7.36-7.43 (2H, m) | 425 (M⁺ − 1) |
| | 7.56-7.60 (1H, t) 7.63-7.69 (1H, q) 7.73-7.75 (2H, d) 7.82-7.85 (1H, d) 7.92-7.93 (1H, d) | |
| | 7.98-8.02 (1H, t) 11.59 (1H, s) 12.38 (1H, s) | |
| 87 | 3.54-3.57 (2H, d) 6.95-6.97 (1H, d) 7.25-7.27 (2H, d) 7.57-7.69 (3H, m) 7.77-7.79 (1H, d) | 404 (M⁺ − 1) |
| | 7.94-8.04 (2H, dd) 8.25-8.28 (1H, d) 9.31-9.39 (1H, d). | |
| 88 | 3.59 (2H, s) 7.03-7.05 (1H, d) 7.30-7.34 (2H, t) 7.50-7.58 (2H, m) 7.76-7.79 (2H, t) | 453 (M⁺ − 1) |
| | 7.82-7.85 (2H, m) 7.90-7.94 (3H, t) 7.98-8.00 (1H, d) 9.47-9.55 (1H, d) 12.08 (1H, s). | |
| 89 | 2.11-2.17 (2H, q) 2.67-2.74 (2H, q) 6.08-6.70 (1H, d) 6.81-6.83 (1H, d) 7.09-7.13 (1h, t) | 401 (M⁺ − Na) |
| | 7.31 (1H, s) 7.46-7.48 (1H, t) 7.50-7.53 (1H, m) 7.60-7.62 (1H, d) 7.83-7.96 (4H, m) | |
| | 8.02 (1H, s). | |
| 90 | 3.39-3.47 (4H, m) 3.59 (2H, d) 3.65 (4H, m) 6.69 (1H, m) 6.94 (1H, m) 7.01 (1H, m) | 498 (M⁺ − 1) |
| | 7.06-7.14 (2H, m) 7.28-7.31 (2H, t) 7.38-7.40 (1H, d) 7.49-7.52 (1H, d) 7.54-7.56 (1H, d) | |
| | 7.61-7.64 (1H, m) 7.71-7.75 (1H, t) 8.13 (1H, s) 11.47 (1H, s) 12.31 (1H, s). | |
| 91 | 3.59 (2H, d) 6.58-6.61 (1H, d) 7.02-7.04 (1H, d) 7.29-7.34 (2H, t) 7.64-7.68 (2H, t) | 403 (M⁺ − 1) |
| | 7.72-7.81 (3H, q) 7.96-7.98 (1H, d) 8.03-8.06 (1H, d) 8.54-8.62 (1H, d) 11.65 (1H, s) | |
| | 12.38-12.44 (1H, bs) | |
| 92 | 3.12-3.16 (2H, s) 6.66 (2H, m) 6.87 (1H, s) 7.03 (1H, m) 7.07-7.09 (3H, m) | 405 (M⁺ − Na) |
| | 7.19-7.29 (2H, d) 7.62-7.67 (2H, d) 7.72 (1H, m) 7.79 (1H, s). | |
| 93 | 3.98 (2H, s) 5.30-5.34 (2H, d) 6.87-6.89 (1H, d) 7.19-7.21 (2H, d) 7.32-7.39 (1H, m) | 517 (M⁺ − Na) |
| | 7.42-7.49 (3H, m) 7.51-7.55 (4H, m) 7.76-7.83 (1H, m) 7.88-7.92 (2H, m) 7.93-7.98 (1H, m) | |
| | 8.04 (1H, d). | |
| 94 | 3.01-3.07 (1H, dd) 3.51 (2H, s) 3.61-3.62 (1H, dd) 4.74-4.77 (1H, m) 6.87-6.88 (1H, d) | 415 (M⁺ − 1) |
| | 7.19-7.21 (1H, d) 7.24-7.26 (1H, d) 7.31-7.38 (3H, m) 7.43-7.46 (2H, m) 7.58-7.60 (2H, d) | |
| | 7.63-7.65 (3H, m) 11.14 (1H, s) 12.14 (1H, s). | |
| 95 | 3.01-3.07 (1H, dd) 3.43 (2h, s) 3.59-3.64 (1H, dd) 4.74-4.77 (1H, m) 6.58-6.60 (1H, d) | 415 (M⁺ − Na) |
| | 6.98-7.00 (1H, d) 7.24-7.26 (1H, d) 7.30-7.33 (2H, m) 7.40-7.44 (3H, m) 7.50-7.52 (2H, d) | |
| | 7.60-7.64 (3H, m). | |
| 96 | 2.16-2.20 (2H, t) 2.64-2.74 (2H, t) 5.29-5.31 (2H, d) 6.72-6.74 (1H, d) 6.98-7.00 (1H, d) | 507 (M⁺ − Na) |
| | 7.06-7.07 (1H, d) 7.22-7.24 (1H, d) 7.35-7.40 (2H, m) 7.43-7.46 (4H, m) 7.53-7.56 (2H, t) | |
| | 7.63-7.68 (1H, d) 7.72-7.78 (2H, m) 7.85-7.87 (1H, d). | |
| 97 | 4.38 (2H, s) 6.26-6.29 (1H, d) 7.17-7.22 (2H, d) 7.33-7.39 (2H, m) 7.45-7.49 (2H, m) | 452 (M⁺ − Na) |
| | 7.51-7.53 (3H, d) 7.63-7.65 (2H, d) 7.68-7.71 (2H, d) 7.74-7.76 (1H, d). | |
| 98 | 3.86 (3H, s) 3.94 (2H, s) 6.70-6.72 (2H, d) 7.00-7.02 (1H, d) 7.08-7.11 (2H, dd) | 441 (M⁺ − Na) |
| | 7.32 (1H, s) 7.54-7.56 (1H, dd) 7.78-7.80 (2H, d) 7.83-7.86 (2H, d). | |
| 100 | 3.58 (1H, s) 6.78 (1H, s) 7.31-7.33 (2H, d) 7.39-7.42 (2H, t) 7.48-7.50 (2H, t) | 397 (M⁺ − 1) |
| | 7.65-7.67 (2H, d) 7.75-7.81 (4H, m) 7.96-7.98 (2H, d) 11.78 (1H, s) 12.26 (1H, s). | |
| 101 | 3.30 (2H, s) 6.01 (1H, s) 7.03-7.05 (2H, d) 7.18-7.20 (2H, d) 7.34-7.38 (1H, t) | 399 (M⁺ − Na) |
| | 7.45-7.49 (2H, t) 7.69-7.72 (4H, t) 7.80-7.82 (2H, d). | |
| 102 | DMSO-d6: d 3.15-3.19 (2H, s) 6.71-6.73 (1H, bs) 7.04-7.06 (2H, d) 7.25 (1H, d) | 552 (M⁺ − Na) |
| | 7.34-7.35 (4H, m) 7.43-7.47 (2H, m) 7.52-7.54 (2H, m) 7.62-7.79 (7H, m) 11.63 (1H, s) | |
| | 12.42-12.49 (1H, bs). | |
| 103 | DMSO-d6: d 3.21 (2H, s) 6.55-6.58 (1H, d) 6.82-6.86 (2H, d) 7.10-7.12 (2H, bs) | 542 (M⁺ − Na) |
| | 7.35 (3H, s) 7.43-7.44 (1H, b) 7.58-7.60 (2H, d) 7.70 (3H, s) 7.76-7.79 (1H, d) | |
| | 7.89-8.00 (1H, dd) 8.49-8.59 (1H, d). | |
| 104 | DMSO-d6: d 2.23-2.32 (3H, s) 3.13-3.16 (2H, d) 5.29-5.31 (2H, s) 6.68-6.70 (1H, d) | 646 (M⁺ − Na) |
| | 6.94-6.96 (1H, d) 7.01-7.03 (1H, d) 7.11-7.13 (2H, t) 7.22-7.27 (1H, m) 7.35-7.38 (2H, m) | |
| | 7.42-7.46 (4H, t) 7.53-7.60 (4H, m) 7.64 (1H, s) 7.69-7.78 (2H, m) 7.84-7.87 (1H, m). | |
| 105 | DMSO-d6: d 3.14-3.18 (2H, s) 6.69-6.71 (1H, d) 6.95-6.97 (1H, m) 7.02-7.04 (1H, d) | 586 (M⁺ − Na) |
| | 7.20 (1H, m) 7.27 (1H, m) 7.34-7.39 (1H, d) 7.42-7.43 (2H, m) 7.45-7.47 (2H, m) | |
| | 7.51-7.53 (2H, d) 7.60-7.62 (1H, d) 7.65-7.72 (5H, m) 7.74-7.78 (1H, m). | |
| 106 | DMSO-d6: d 3.21 (2H, s) 3.87-3.90 (3H, d) 6.85-6.86 (1H, bs) 7.11-7.13 (3H, t) | 590 (M⁺ − Na) |
| | 7.30 (1H, s) 7.39-7.41 (2H, d) 7.55-7.57 (2H, d) 7.66-7.72 (3H, t) 7.82-7.84 (1H, d) | |
| | 7.89-7.94 (1H, d) | |
| 107 | DMSO-d6: d 3.23 (2H, s) 6.57-6.60 (1H, d) 6.91 (1H, s) 7.17 (2H, s) 7.39-7.41 (2H, d) | 576 (M⁺ − Na) |
| | 7.62-7.64 (2H, d) 7.70-7.72 (3H, t) 7.77-7.81 (2H, d) 7.93-7.95 (1H, d) 8.01-8.04 (1H, d) | |
| | 8.52-8.61 (1H, d) | |
| 108 | DMSO-d6: d 1.23 (3H, s) 1.34-1.43 (15H, d) 2.36 (3H, s) 3.46 (2H, s) 6.99 (1H, bs) | 618 (M⁺ − 1) |
| | 7.18-7.20 (2H, d) 7.26-7.29 (1H, d) 7.33-7.37 (2H, t) 7.42 (1H, s) 7.68-7.69 (2H, t) | |
| | 7.72-7.74 (2H, d) 11.45 (1H, s) 12.19-12.33 (1H, bs). | |
| 109 | 3.58-3.59 (2H, d) 7.01 (1H, bs) 7.29-7.30 (2H, m) 7.40 (1H, bs) 7.47-7.51 (2H, m) | 489 (M − 1)⁻ |
| | 7.62-7.64 (2h, d) 7.72-7.74 (3H, m) 7.79-7.82 (4H, m) 7.87-7.88 (2H, m) 7.93-7.95 (1H, d) | |
| | 12.33-12.51 (1H, bs). | |
| 110 | 3.60 (2H, s), 7.01-7.03 (1H, m), 7.08-7.10 (2H, d), 7.22-7.24 (1H, m), | 467 (M − 1)⁻ |
| | 7.29-7.33 (1H, m), 7.56-7.58 (1H, m), 7.63-7.67 (1H, m), 7.84-7.91 (2H, m), | |
| | 7.93-7.96 (1H, s), 8.124 (1H, d), 11.64 (1H, s) | |
| 111 | 7.59-7.66 (3H, m), 7.69-7.70 (1H, d), 7.71-7.72 (1H, d), 7.95 (1H, s), | 403 (M + 1) |
| | 7.97-8.02 (2H, m), 8.04-8.07 (3H, m), 8.20 (1H, s), 12.68 (1H, s). | |
| 112 | 2.27 (3H, s), 3.16-3.19 (2H, s), 6.71-6.73 (1H, d), 6.96-6.98 (1H, d), 7.04-7.06 (1H, d), | 564 (M + 1) |
| | 7.15-7.19 (2H, d), 7.24-7.26 (1H, d), 7.33-7.38 (1H, d), 7.46-7.48 (1H, q), | |
| | 7.51-7.54 (1H, t), 7.60-7.63 (2H, q), 7.84-7.87 (2H, q), 7.89-7.93 (1H, d), 7.94-7.98 (1H, m), | |
| | 11.60 (1H, s). | |
| 113 | 3.44 (2H, s), 5.27-5.29 (2H, d), 6.79-6.81 (1H, d), 7.06-7.14 (2H, dd), | 511 (M − 23)⁻ |
| | 7.25-7.34 (3H, m), 7.40-7.41 (1H, m), 7.44-7.48 (2H, m), 7.59-7.64 (2H, m), 7.70-7.78 (2H, m), | |
| | 7.85-8.03 (2H, m), 11.63 (1H, s). | |
| 114 | 2.55-2.58 (3H, d), 4.33 (2H, s), 5.31-5.35 (2H, d), 6.94-6.98 (1H, m), | 542 (M − 1) |
| | 7.38-7.39 (2H, dd), 7.43-7.45 (3H, dd), 7.53-7.60 (5H, m), 7.78-7.85 (3H, m), | |
| | 7.92-7.99 (1H, m), 8.06-8.10 (1H, m), 11.5 (1H, s), 11.73-11.82 (1H, s) | |
| 115 | 3.14-3.16 (2H, s), 5.18-5.20 (2H, s), 6.66-6.68 (1H, d), 6.93-6.97 (1H, t), | 443 (M − 23) |
| | 7.01-7.05 (1H, t), 7.07-7.09 (2H, d), 7.16-7.24 (2H, m), 7.33-7.36 (1H, t), | |
| | 7.40-7.43 (3H, t), 7.46-7.49 (2H, d), 7.54-7.56 (1H, s), 11.64 (1H, s) | |
| 116 | 3.12-3.14 (2H, d), 3.95-3.97 (3H, d), 6.68-6.70 (1H, d), 7.02-7.04 (1H, d), | 417 (M − 23) |
| | 7.08-7.10 (1H, d), 7.23-7.28 (1H, m), 7.29-7.37 (1H, m), 7.39-7.47 (1H, m), | |
| | 7.57--7.64 (1H, m), 7.73-7.75 (1H, m), 7.78-7.81 (1H, m), 7.85-7.87 (1H, m), 7.99 (1H, s), | |
| | 11.63 (1H, s) | |
| 117 | 1.45-1.48 (3H, t), 3.59 (2H, s), 4.23-4.25 (2H, q), 6.68-6.70 (1H, dd), | 431 (M − 23) |
| | 7.02-7.04 (1H, dd), 7.08-7.10 (2H, d), 7.23-7.26 (1H, m), 7.28-7.30 (1H, m), | |
| | 7.34-7.39 (1H, m), 7.41--7.44 (1H, m), 7.65-7.75 (1H, m), 7.77-7.82 (1H, m), | |
| | 7.83-7.85 (1H, m), 11.63 (1H, s) | |
| 118 | 2.28-2.30 (3H, s), 3.11-3.15 (2H, s), 5.18-5.21 (2H, s), 6.65-6.67 (1H, d), | 620 (M + 1) |
| | 6.92-6.95 (2H, m), 6.99-7.01 (1H, d), 7.05-7.06 (1H, m), 7.09-7.14 (2H, m), 7.17-7.22 (2H, m), | |
| | 7.35-7.36 (1H, d), 7.40-7.44 (3H, m), 7.46-7.48 (2H, d), 7.54-7.57 (2H, d), 7.59-7.61 (1H, s), | |
| | 11.64 (1H, s) | |
| 119 | 3.11-3.18 (2H, s), 5.30-5.33 (2H, s), 6.24-6.27 (2H, m), 6.40-6.41 (1H, d), | 508 (M − 23) |
| | 6.64-6.67 (1H, d), 6.94-6.98 (1H, m), 7.00-7.02 (2H, m), 7.19-7.24 (2H, m), | |
| | 7.36-7.37 (2H, m), 7.41-7.46 (4H, m), 7.48-7.51 (2H, m), 11.64 (1H, s) | |
| 120 | 3.38-3.39 (2H, s), 5.27-5.30 (2H, s), 6.64-6.66 (1H, d), 6.96-7.02 (2H, q), | 544 (M − 23) |
| | 7.06-7.09 (2H, m), 7.12 (1H, s), 7.17-7.26 (1H, m), 7.41-7.48 (1H, m), 7.57-7.61 (2H, d), | |
| | 7.63-7.64 (4H, s), 7.68-7.70 (1H, d), 7.78-7.82 (1H, T), 7.95-7.97 (1H, d), 11.64 (1H, s) | |
| 121 | 3.58 (2H, s), 6.09-6.14 (2H, d), 6.98-6.99 (1H, d), 7.05 (1H, s), 7.09-7.12 (1H, d), | 381 (M − 1) |
| | 7.16-7.19 (1H, d), 7.27-7.29 (2H, d), 7.61 (1H, s), 7.66-7.67 (1H, d), 11.54 (1H, s), | |
| | 12.20-12.45 (1H, bs). | |
| 123 | 3.58 (2H, bs), 4.25-4.31 (4H, m), 6.96-7.02 (3H, t), 7.13 (1H, s), 7.27-7.31 (2H, m), | 395.1 (M − 1) |
| | 7.49 (1H, s), 7.67-7.69 (1H, d), 11.55 (1H, s), 11.76-12.29 (1H, bs). | |
| 124 | 3.51-3.55 (2H, s), 6.32-6.33 (2H, d), 6.83 (1H, s), 6.91-6.98 (3H, m), | 468 (M − 1) |
| | 7.10-7.16 (2H, m), 7.19-7.25 (2H, m), 7.36-7.41 (3H, m), 7.52-7.54 (1H, d), 7.91 (1H, s), | |
| | 11.4 (1H, s), 12.5 (1H, bs) | |
| 125 | 2.38 (3H, s), 3.54 (2H, s), 4.25-4.31 (4H, m), 6.96-7.03 (3H, m), 7.13-7.21 (3H, m), | 548 (M − 1) |
| | 7.39-7.41 (2H, d), 7.53 (1H, s), 7.63-7.68 (1H, t), 7.78-7.79 (2H, bs), 11.52 (1H, s), | |
| | 12.19-12.46 (1H, bs). | |
| 126 | 2.37 (3H, s), 3.51 (2H, s), 6.09-6.14 (2H, d), 6.96-7.12 (3H, m), 7.17-7.19 (3H, d), | 536 (M + 1) |
| | 7.37-7.38 (2H, d), 7.57 (1H, s), 7.66 (1H, s), 7.76 (2H, bs), 11.54 (1H, s), | |
| | 12.24-12.48 (1H, bs). | |
| 128 | 3.75 (2H, s), 7.25 (1H, s), 7.62-7.64 (2H, t), 7.72-7.78 (1H, t), 7.85 (1H, s), | 394 (M − 1) |
| | 7.94-7.99 (1H, m), 8.06-8.09 (2H, d), 8.24 (1H, s), 12.4-12.54 (1H, s), 12.72 (1H, s) | |
| 129 | 3.15 (2H, s), 3.28 (2H, t), 4.32 (2H, t), 6.70-6.98 (1H, d), 7.03-7.05 (1H, dd), | 507 (M − 23) |
| | 7.08-7.10 (1H, d), 7.22-7.27 (2H, m), 7.32-7.37 (4H, m), 7.40-7.44 (2H, m), 7.65 (1H, s), | |
| | 7.71-7.74 (2H, m), 7.78-7.84 (1H, m), 7.98 (1H, s), 11.63 (1H, s) | |
| 130 | 3.70-3.74 (2H, s), 7.25 (1H, s), 7.40-7.44 (1H, t), 7.49-7.53 (2H, t), | 420 (M − 1) |
| | 7.69-7.79 (6H, m), 7.85-7.90 (1H, t), 12.52 (1H, s), 12.70 (1H, s) | |
| 131 | 2.27-2.30 (3H, s), 3.13-3.16 (2H, s), 5.23 (2H, s), 6.69-6.71 (1H, d), 7.03-7.05 (1H, d), | 664 (M − 1) |
| | 7.11-7.15 (2H, d), 7.28-7.21 (1H, s), 7.23-7.25 (2H, d), 7.28 (1H, s), | |
| | 7.30-7.33 (3H, m), 7.34-7.37 (1H, m), 7.39-7.42 (1H, d), 7.48-7.58 (1H, d), 7.60-7.66 (2H, d), | |
| | 7.74-7.76 (1H, d), 8.54 (1H, S), 11.63 (1H, s) | |
| 132 | 2.30-2.39 (3H, s), 3.46 (2H, s), 6.61 (1H, s), 6.86-6.88 (1H, d), 7.17-7.22 (2H, s), | 419 (M + 1) |
| | 7.41-7.42 (1H, s), 7.45-7.50 (3H, s), 7.55-7.59 (2H, d), 7.62-7.64 (1H, d), 11.5 (1H, s), | |
| | 12.3 (1H, bs) | |
| 133 | 3.71 (2H, s), 5.34 (2H, s), 7.24 (1H, s), 7.44-7.60 (8H, m), 7.83 (1H, m), | 500 (M − 1) |
| | 7.99-8.09 (3H, m), 12.49 (1H, s), 12.67-12.70 (1H, s) | |
| 134 | 2.46 (3H, s), 3.68 (2H, s), 6.71 (1H, s), 7.21 (1H, s), 7.46-7.50 (1H, t), 7.57 (1H, s), | 426 (M + 1) |
| | 7.60-7.63 (2H, t), 7.70-7.72 (2H, d), 11.48 (1H, s), 12.3 (1H, s) | |
| 135 | 1.35-1.43 (2H, m), 1.58-1.61 (2H, m), 1.63-1.68 (2H, q), 1.85-1.88 (2H, q), | 485 (M − 23) |
| | 3.10-3.13 (1H, m), 3.15 (2H, s), 4.02-4.05 (2H, t), 6.68-6.70 (1H, d), 7.02-7.04 (1H, d), | |
| | 7.08-7.10 (1H, d), 7.22-7.26 (1H, m), 7.27-7.33 (1H, m), 7.34-7.43 (1H, m), 7.65-7.68 (1H, m), | |
| | 7.71-7.76 (2H, m), 7.79-7.85 (1H, m), 7.99 (1H, s), 11.62 (1H, s) | |
| 136 | 3.26-3.30 (2H, q), 3.32-3.38 (1H, m), 5.29-5.31 (2H, d), 6.66-6.69 (1H, d), | 583 (M − 23) |
| | 7.05-7.10 (2H, m), 7.15-7.17 (4H, m), 7.27-7.31 (1H, m), 7.35-7.40 (2H, m), | |
| | 7.42-7.46 (4H, m), 7.53-7.56 (2H, m), 7.63-7.68 (1H, m), 7.71-7.78 (2H, m), | |
| | 7.85-7.87 (1H, d), 8.02 (1H, s), 11.61 (1H, s) | |
| 137 | 3.57-3.58 (2H, s), 6.98-6.99 (1H, d), 7.26-7.35 (3H, q), 7.37-7.42 (1H, t), | 355 (M − 1) |
| | 7.56-7.60 (2H, m), 7.67-7.69 (2H, m), 11.64 (1H, s), 12.17-12.29 (1H, bs) | |
| 138 | 1.03-1.08 (3H, t), 1.82-1.90 (2H, m), 3.58 (2H, s), 4.10-4.16 (2H, q), 7.02-7.04 (1H, d), | 447 (M + 1) |
| | 7.28-7.30 (1H, d), 7.32-7.34 (1H, s), 7.40-7.42 (2H, m), 7.47-7.49 (1H, m), | |
| | 7.51-7.55 (1H, m), 7.72-7.74 (1H, d), 7.79-7.92 (2H, m), 7.98-8.09 (1H, m), 11.63 (1H, s), | |
| | 12.38 (1H, s) | |
| 139 | 1.13-1.2 (6H, d), 1.80-1.91 (2H, m), 2.60-2.79 (2H, t), 3.10-3.40 (2H, t), 3.57 (2H, s), | 434 (M − 1)⁻ |
| | 4.13-4.19 (1H, m), 6.8 (1H, m), 7.00-7.08 (1H, d), 7.14-7.18 (1H, s), 7.28 (2H, s), | |
| | 7.40-7.50 (1H, d), 7.70 (1H, s), 11.50 (1H, bs), 11.90-12.30 (1H, bs) | |
| 140 | 0.90-1.09 (6H, t), 1.80-1.90 (4H, m), 3.56 (2H, s), 4.09-4.15 (4H, t), 6.90-7.02 (2H, m), | 503 (M − 1) |
| | 7.20-7.30 (2H, m), 7.40-7.49 (2H, dd), 7.60-7.68 (2H, dd), 7.79 (1H, s), | |
| | 7.80-8.03 (1H, t), 11.56 (1H, s), 11.80-12.60 (1H, bs) | |
| 141 | 1.84-1.87 (2H, m), 2.60-2.70 (4H, t), 2.89 (3H, s), 3.50 (2H, s), 6.63-6.66 (1H, m), | 406 (M − 1) |
| | 6.99-7.03 (2H, dd), 7.10 (1H, bs), 7.20 (2H, s), 7.50 (1H, s), 7.70-7.72 (1H, dd), | |
| | 11.30 (1H, s), 12.26 (1H, s) | |
| 142 | 1.74-1.81 (1H, m), 1.87-1.96 (2H, m), 2.04-2.08 (1H, m), 3.58 (2H, s), | 489 (M + 1) |
| | 3.71-3.77 (1H, m), 3.83-3.86 (1H, m), 4.09-4.20 (2H, m), 4.30-4.32 (1H, m), | |
| | 7.01-7.03 (1H, d), 7.27-7.29 (1H, d), 7.31-7.36 (1H, d), 7.42-7.44 (2H, m), 7.51-7.55 (1H, m), | |
| | 7.71-7.73 (1H, d), 7.78-7.85 (2H, m), 7.87-7.93 (1H, m), 8.03-8.07 (1H, m), | |
| | 11.65 (1H, s), 12.37 (1H, s) | |
| 143 | 1.30-1.40 (3H, t), 3.30-3.34 (2H, s), 4.10-4.17 (2H, q), 6.76-6.78 (2H, dd), | 455 (M + 1) |
| | 7.07 (1H, s), 7.14-7.18 (2H, dd), 7.20-7.30 (3H, t), 7.77-7.78 (2H, m), 7.86 (1H, s), | |
| | 11.30 (1H, s) | |
| 144 | 1.43-1.48 (6H, t), 3.58 (2H, s), 4.19-4.26 (4H, q), 6.90-7.03 (2H, m), 7.20-7.30 (2H, m), | 475 (M − 1) |
| | 7.41 (1H, dd), 7.50 (1H, s), 7.72-7.74 (1H, dd), 7.79 (1H, s), 7.95-7.98 (1H, d), | |
| | 8.06 (1H, s), 11.60 (1H, s), 12.38-12.42 (1H, bs) | |
| 145 | 3.12-3.15 (2H, d), 6.70-6.72 (1H, d), 7.00-7.03 (1H, d), 7.08-7.10 (1H, d), | 420 (M − 23) |
| | 7.19-7.24 (1H, t), K 7.48-7.58 (3H, m), 7.62 (1H, s), 7.76 (1H, s), 7.88-7.89 (1H, d), | |
| | 8.05-8.06 (1H, d), 11.62 (1H, s) | |
| 146 | 3.5 (2H, s), 3.90-3.94 (3H, d), 7.00- (1H, dd), 7.20-7.34 (2H, dd), 7.50-7.53 (1H, dd), | 417 (M − 1) |
| | 7.63-7.68 (3H, m), 7.72-7.74 (1H, dd), 7.78-7.80 (1H, dd), 7.90 (1H, s), | |
| | 8.130-8.15 (1H, m), 11.60 (1H, bs), 12.39 (1H, bs) | |
| 147 | 3.23 (2H, s) 6.70-6.71 (1H, m) 7.05-7.12 (2H, m) 7.23 (2H, s) 7.66-7.76 (2H, m) | 405 (M − 23) |
| | 7.89-7.97 (2H, m), 10.11 (1H, s), 11.6 (1H, s). | |
| 148 | 3.25 (2H, s), 6.25-6.26 (1H, t), 6.29 (1H, s), 6.71-6.73 (1H, d), 7.04-7.06 (1H, d), | 402 (M − 23) |
| | 7.10-7.12 (1H, d), 7.17 (1H, s), 7.25-7.27 (1H, d), 7.36-7.37 (1H, t), 7.44-7.45 (1H, t), | |
| | 7.50-7.52 (1H, d), 7.58-7.62 (2H, m), 7.69 (1H, d), 11.59 (1H, s) | |
| 149 | 3.20-3.22 (2H, d), 5.20-5.25 (2H, d), 7.15-7.17 (1H, m), 7.19-7.21 (1H, m), | 519 (M − 23) |
| | 7.27-7.29 (2H, m), 7.32-7.35 (2H, m), 7.37-7.39 (2H, m), 7.41-7.44 (3H, m), | |
| | 7.45-7.49 (3H, m), 7.55 (1H, s), 7.60-7.62 (1H, d), 7.65-7.66 (1H, m), 7.72 (1H, m), 11.64 (1H, s) | |
| 150 | 1.46-1.49 (3H, t), 3.10 (2H, s), 4.00-4.09 (2H, q), | 431 (M − 23) |
| | 6.66-6.68 (1H, dd), 6.80 (1H, s), 7.00 (1H, dd), 7.20-7.22 (1H, dd), | |
| | 7.52-7.54 (2H, m), 7.60 (1H, s), 7.67 (1H, s), 7.70 (1H, s), 7.80-7.85 (1H, dd), | |
| | 8.06-8.10 (1H, m), 11.30 (1H, s) | |
| 151 | 3.15 (2H, s), | 493 (M − 23) |
| | 4.94-4.98 (2H, d), 6.60 (1H, dd), 7.10 (1H, dd), 7.15 (1H, dd), 7.21 (1H, dd), | |
| | 7.43-7.60 (5H, m), 7.63-7.65 (3H, dd), 7.78-7.80 (1H, s), 7.83-7.85 (1H, s), | |
| | 7.91-7.93 (1H, m), 8.15-8.30 (1H, dd), 11.60 (1H, s) | |
| 152 | 3.14 (2H, s), 3.79-3.81 (3H, s), 6.67-6.69 (1H, d), 7.00-7.05 (2H, m), 7.08-7.10 (1H, d), | 443 (M − 23) |
| | 7.16-7.17 (1H, d), 7.21-7.24 (1H, q), 7.45-7.47 (1H, d), 7.50-7.51 (1H, d), 7.54-7.56 (1H, m), | |
| | 7.60-7.62 ((1H, d), 7.64-.7.66 (1H, d), 7.67-7.69 (1H, dd), 7.76-7.78 (1H, m), 11.62 (1H, s) | |
| 153 | 3.15 (2H, s), 6.68-6.70 (1H, d), 7.06-7.07 (1H, m), 7.10-7.12 (2H, d), 7.19-7.21 (1H, t), | 425 (M − 23) |
| | 7.23-7.25 (1H, d), 7.27-7.28 (1H, d), 7.31-7.32 (1H, s), 7.33-7.37 (1H, m), | |
| | 7.48-7.49 (1H, d), 11.60 (1H, s) | |
| 154 | 3.87 (3H, d), 4.17-4.18 (2H, t), 4.21-4.22 (2H, t), 6.71-6.73 (1H, dd), | 455 (M + 1) |
| | 6.90-7.00 (1H, dd), 7.07-7.09 (1H, dd), 7.21-7.23 (1H, dd), 7.40-7.51 (2H, m), 7.60 (1H, s), | |
| | 7.60-7.70 (1H, q), 7.80 (1H, s), 7.92-7.94 (1H, dd), 8.00-8.11 (1H, t), 11.30 (1H, bs) | |
| 155 | 0.70-0.84 (3H, t), 1.44-1.50 (2H, m), 1.86-1.95 (1H, m), 3.87-3.90 (3H, s) | 445 (M − 23) |
| | 6.67-6.69 (1H, dd), 7.08-7.11 (1H, dd), 7.15-7.19 (2H, t), 7.49-7.55 (1H, m), 7.58 (1H, s), | |
| | 7.64-7.66 (1H, s), 7.80 (1H, s), 7.84-7.86 (1H, dd), 7.92-7.94 (1H, dd) | |
| | 8.06-8.11 (1H, t), 11.56 (1H, bs) | |
| 156 | 3.87 (3H, s), 4.36-4.39 (2H, s), 6.21-6.23 (1H, dd), | 456 (M − 23) |
| | 6.60-6.62 (1H, dd), 6.90 (1H, d), 7.13 (1H, s), 7.19 (1H, d), 7.40 (1H, d), 7.50 (1H, d), 7.56 (1H, d), | |
| | 7.60 (2H, s), 7.80-7.82 (1H, t), 8.00-8.11 (1H, d), 11.30 (1H, bs). | |
| 157 | 3.16 (2H, s), 5.29-5.31 (2H, s), 6.67-6.69 (1H, d), 7.02-7.04 (1H, d), | 623 (M − 23) |
| | 7.09-7.15 (2H, dd), 7.20-7.22 (1H, d), 7.37-7.45 (1H, m), 7.49-7.50 (1H, s), | |
| | 7.58-7.65 (6H, m), 7.68-7.70 (1H, d), 7.79-7.82 (1H, t), 7.96-7.98 (1H, d), 11.62 (1H, s). | |
| 158 | 2.26-2.33 (3H, s), 3.13-3.20 (2H, s), 3.77-3.81 (3H, s), 6.68-6.70 (1H, d), | 620 (M + 1) |
| | 6.94-7.04 (4H, t), 7.13-7.16 (3H, t), 7.20-7.23 (1H, d), 7.47-7.49 (1H, d), 7.59-7.67 (6H, q), | |
| | 7.69-7.72 (1H, q), 11.60 (1H, s) | |
| 159 | 3.77-3.81 (3H, s), 4.36 (2H, s), 6.17-6.22 (1H, d), 6.58-6.61 (1H, dd), | 482 (M − 23) |
| | 6.94-6.98 (1H, d), 7.00-7.05 (1H, dd), 7.12-7.18 (3H, m), 7.41-7.50 (2H, m), | |
| | 7.57-7.62 (3H, q), 7.64-7.76 (2H, m) | |
| 160 | 3.21-3.23 (2H, t), 3.28 (2H, s), 4.49-4.55 (2H, t), 6.71-6.73 (1H, d), 6.77-6.79 (1H, d), | 378.9 (M − 23) |
| | 7.03-7.05 (1H, d), 7.09-7.12 (2H, m), 7.18-7.20 (1H, m), 7.27-7.30 (2H, m), 11.63 (1H, s). | |
| 161 | 3.10 (2H, s), 4.28-4.31 (3H, s), 6.67-6.69 (1H, d), 6.87 (1H, s), 7.01-7.03 (1H, m), | 429 (M − NaCl) |
| | 7.07-7.09 (2H, m), 7.23 (1H, m), 7.67-7.69 (1H, d), 7.76-7.80 (1H, m), 8.12-8.14 (1H, d), | |
| | 8.18-8.20 (1H, d), 8.44-8.46 (1H, d), 8.89-8.97 (2H, m), | |
| 162 | 3.59 (2H, s), 5.50-5.54 (2H, d), 7.28-7.29 (1H, m), 7.32-7.37 (1H, m), | 538 (M − 1) |
| | 7.43-7.47 (1H, m), 7.49-7.55 (1H, m), 7.57-7.63 (1H, m), 7.69-7.74 (1H, m), | |
| | 7.78-7.84 (3H, q), 7.91-7.93 (1H, m), 7.94-8.00 (1H, m), 8.02-8.06 (1H, t), | |
| | 8.08-8.15 (1H, m), 8.30-8.32 (2H, m), 11.66 (1H, s), 12.39-12.60 (1H, bs) | |
| 163 | 3.60-3.61 (2H, d), 4.84-4.86 (2H, t), 7.24-7.36 (4H, m), 7.56-7.58 (1H, d), | 461 (M − 1) |
| | 7.69-7.77 (2H, m), 7.86-7.97 (2H, m), | |
| | 8.06-8.08 (1H, s), 8.14 (1H, s), 11.63 (1H, s), 12.40 (1H, bs), 13.14 (1H, bs). | |
| 164 | 3.13 (2H, s), 3.80 (3H, s), 3.91 (3H, s), 6.68-6.70 (1H, d), 7.02-7.04 (1H, d), | 471 (M + 1) |
| | 7.08-7.11 (2H, d), 7.16-7.17 (1H, d), 7.23 (1H, d), 7.30 (1H, s), 7.60 (1H, s), 7.70 (1H, s), | |
| | 7.90 (1H, s), 11.50 (1H, bs) | |
| 165 | 3.40 (2H, s), 6.80-6.82 (1H, d), 7.08-7.10 (1H, d), 7.15-7.16 (1H, d), 7.34-7.36 (1H, d), | 429 (M + 1) |
| | 7.39-7.42 (1H, d), 7.40-7.47 (1H, t), 7.64-7.67 (1H, d), 7.73-7.75 (1H, d), 7.89-7.91 (1H, d), | |
| | 7.96-7.98 (1H, s), 8.04-8.08 (1H, m), 11.50 (1H, bs) | |
| 166 | 3.20 (2H, s), 3.94 (3H, s), 3.98 (3H, s), 6.70-6.72 (1H, d), 7.03 (1H, d), 7.09 (1H, d), | 471 (M + 1) |
| | 7.19-7.21 (1H, t), 7.23-7.25 (1H, t), | |
| | 7.30-7.32 (1H, d), 7.42 (1H, s), 7.50 (1H, s), 7.78 (1H, s), 7.90 (1H, s) | |
| 167 | 2.33 (3H, s), 3.12-3.15 (2H, s), 5.23-5.25 (2H, d), 6.68-6.70 (1H, d), 7.02-7.04 (1H, d), | 507 (M − 23) |
| | 7.08-7.10 (1H, d), 7.22-7.24 (3H, m), 7.25-7.27 (1H, d), 7.40 (1H, m), 7.42-7.46 (4H, m), | |
| | 7.72-7.76 (2H, m), 7.84 (1H, s), 11.60 (1H, s) | |
| 168 | 2.24 (3H, s), 2.30-2.34 (3H, s), 3.14-3.17 (2H, s), 5.22-5.26 (2H, d), 6.69-6.71 (1H, d), | 660 (M − 23) |
| | 6.95-6.97 (1H, d), 7.02-7.04 (1H, d), 7.12-7.15 (2H, t), 7.24-7.25 (4H, d), 7.35-7.43 (5H, m), | |
| | 7.58-7.62 (2H, t), 7.67-7.78 (2H, m), 7.87 (1H, s), 11.60 (1H, s). | |
| 169 | 3.22 (2H, s), 5.48-5.50 (2H, d), 6.71-6.73 (1H, d), 7.03-7.05 (1H, d), 7.08-7.11 (2H, d), | 498 (M − 23) |
| | 7.25-7.29 (1H, d), 7.31-7.32 (1H, m), 7.37-7.40 (1H, m), 7.42-7.44 (1H, m), | |
| | 7.46-7.54 (1H, m), 7.58-7.60 (1H, m), 7.74-7.76 (1H, m), 7.78-7.80 (1H, m), 7.84-7.88 (1H, m), | |
| | 8.02 (1H, s), 11.63 (1H, s) | |
| 170 | 3.55-3.57 (2H, d), 6.53-6.56 (1H, d), 6.98-6.99 (1H, d), 7.25-7.28 (3H, d), | 375.97 (M − 1) |
| | 7.36-7.55 (3H, m), 7.67 (1H, s), 7.74 (1H, s), 11.13-11.21 (1H, d), 11.65 (1H, s), | |
| | 12.08-12.16 (1H, bs) | |
| 171 | 1.18-1.23 (3H, t), 3.68-3.69 (2H, s), 4.08-4.12 (2H, q), 7.04-7.06 (1H, bs), | 415 (M − 1) |
| | 7.31-7.35 (2H, t), 7.56-7.62 (3H, m), 7.74-7.76 (1H, m), 7.80 (1H, s), 7.89-7.93 (1H, t), | |
| | 7.96-8.01 (2H, m), 8.07-8.11 (1H, t), 11.64 (1H, s) | |
| 172 | 2.08-2.18 (2H, t), 2.64-2.73 (2H, t), 6.70-6.72 (1H, d), 6.97-6.99 (1H, d), | 427 (M − 23) |
| | 7.18-7.25 (2H, q), 7.35-7.39 (1H, q), 7.45-7.48 (2H, m), 7.50-7.54 (1H, d), 7.60-7.62 (1H, d), | |
| | 7.66-7.71 (3H, m), 7.72-7.74 (1H, d), 7.76-7.78 (1H, d), 11.60 (1H, s) | |
| 173 | 2.29 (3H, s), 3.12 (2H, s), 3.17 (2H, s), 3.62 (6H, m), 6.66-6.68 (2H, d), | 651 (M − 23) |
| | 6.88-6.90 ((1H, d), 6.92-6.94 (1H, m), 7.01 (3H, m), 7.06-7.09 ((1H, m), 7.13-7.15 (2H, d), | |
| | 7.21-7.23 (1H, m), 7.31-7.33 (1H, d), 7.39-7.41 (1H, m), | |
| | 7.55-7.61 (3H, m), 8.14 (1H, s), 11.64 (1H, s) | |
| 174 | 2.33-2.35 (3H, s), 3.12-3.15 (2H, s), 6.67-6.69 (1H, d), 7.01-7.03 (1H, d), | 427 (M − 23) |
| | 7.08-7.10 (1H, d), 7.16-7.18 (1H, s), 7.21-7.29 (2H, m), 7.49-7.51 (1H, d), | |
| | 7.54-7.55 (2H, m), 7.57-7.62 (1H, t), 7.64-7.69 (2H, m), 7.71-7.75 (1H, m), 11.65 (1H, s) | |
| 175 | 1.22-1.26 (6H, d), 3.04-3.08 (1H, m), 3.12 (2H, s), 6.56-6.61 (1H, m), | 447 (M − 23) |
| | 6.66-6.68 (1H, d), 6.86-6.90 (1H, m), 6.97-7.04 (1H, d), 7.07-7.09 (1H, d), | |
| | 7.21-7.23 (1H, m), 7.53-7.64 (1H, m), 7.70-7.77 (1H, m), 7.93-7.97 (1H, m), 11.64 (1H, s). | |
| 176 | 2.24-2.30 (3H, d), 3.16-3.18 (2H, d), 3.93-3.99 (3H, d), 6.75-6.77 (1H, d), | 570 (M − 23) |
| | 7.05-7.07 (2H, d), 7.11-7.15 (2H, t), 7.26-7.29 (1H, t), 7.34-7.40 (3H, m), 7.58-7.60 (2H, d), | |
| | 7.73-7.78 (1H, m), 7.82-7.84 (1H, d), 7.87-7.89 (1H, d), 8.00 (1H, s), 11.62 (1H, s) | |
| 177 | 2.34-2.40 (2H, m), 2.71-2.78 (2H, m), 3.95-3.97 (3H, d), 6.75-6.77 (1H, d), | 431 (M − 23) |
| | 7.02-7.04 (1H, d),7.08-7.10 (1H, d), 7.26-7.28 (1H, d), 7.30-7.36 (1H, d), 7.39-7.45 (1H, m), | |
| | 7.59-7.66 (1H, d), 7.72-7.74 (1H, m), 7.76-7.81 (1H, m), | |
| | 7.85-7.87 (1H, m), 7.99 (1H, s), 11.63 (1H, s) | |
| 178 | 3.13-3.15 (2H, d), 6.68-6.70 (1H, d), 7.02-7.04 (1H, d), 7.09-7.11 ((2H, d), 7.17 (1H, s) | 431 (M − 23) |
| | 7.22-7.23 (1H, d), 7.32-7.37 (2H, m), 7.39-7.46 (2H, m), 7.48-7.55 (2H, q), | |
| | 7.57-7.61 (1H, d), 11.65-11.68 (1H, bs) | |
| 179 | 3.59 (2H, s), 4.92-4.96 (2H, d), 7.04-7.05 (1H, d), 7.29-7.33 (2H, d), 7.35-7.38 (2H, m), | 461 (M − 1) |
| | 7.50-7.57 (1H, m), 7.73-7.75 (1H, d), 7.79-7.81 (1H, d), 7.86 (1H, s), 7.91-7.95 (1H, m), | |
| | 8.04-8.06 (1H, d), 11.64 (1H, s), 12.44 (1H, s), 13.12 (1H, s) | |
| 180 | 3.17-3.19 (2H, d), 3.88 (3H, s), 5.24-5.27 (2H, d), 6.70-6.72 (1H, d), 7.02-7.05 (2H, m), | 547 (M + 1) |
| | 7.10-7.12 (2H, d), 7.23-7.30 (1H, m), 7.38-7.42 (3H, m), 7.46-7.50 (2H, m), | |
| | 7.62-7.67 (1H, d), 7.74-7.81 (2H, m), 7.85 (1H, s), 11.60 (1H, s) | |
| 181 | 3.15-3.16 (2H, s), 3.81 (3H, s), 5.27-5.30 (2H, d), 6.67-6.69 (1H, d), 6.92-6.93 (1H, d), | 423 (M − 23) |
| | 7.02-7.04 (1H, d), 7.09-7.10 (2H, d), 7.14-7.15 (1H, d), 7.24-7.30 (2H, m), | |
| | 7.33-7.37 (2H, m), 7.41-7.46 (2H, m), 7.74-7.79 (2H, m), 7.86-7.88 (1H, d), 11.61 (1H, s) | |
| 182 | 3.13-3.16 (2H, s), 5.30-5.32 (2H, d), 6.69-6.71 (1H, d), 7.03-7.05 (1H, d), | 526.9 (M − 23) |
| | 7.09-7.11 (1H, d), 7.22-7.24 (1H, d), 7.28-7.30 (1H, m), 7.38-7.42 (2H, m), | |
| | 7.49-7.52 (2H, m), 7.72-7.87 (2H, q), 7.85-7.87 (1H, s), 7.44 (1H, s), | |
| | 7.55-7.57 (2H, t), 11.63 (1H, s) | |
| 183 | 2.18-2.24 (2H, t) 2.34 (3H, s), 2.67-2.73 (2H, t), 5.23-5.25 (2H, d), 6.72-6.74 (1H, d), | 521 (M − 23)⁻ |
| | 6.99-7.01 (1H, d), 7.06-7.08 (1H, d), 7.23-7.25 (3H, d), 7.28-7.30 (1H, d), 7.61-7.66 (1H, d), | |
| | 7.72-7.74 (1H, d), 7.76-7.78 (1H, d), 7.84-7.87 (1H, d), 11.60 (1H, s) | |
| 184 | 2.10-2.17 (2H, t), 2.67-2.73 (2H, t), 5.29-5.31 (2H, d), 6.70-6.72 (1H, d), | 543 (M − 23)⁻ |
| | 6.98-6.99 (1H, d), 7.05-7.07 (1H, d), 7.21-7.23 (1H, d), 7.26-7.30 (1H, m), 7.35-7.44 (3H, d), | |
| | 7.50 (2H, s), 7.54-7.56 (2H, d), 7.73-7.77 (2H, m), 7.84-7.86 (1H, d), 11.60 (1H, s) | |
| 185 | 3.58-3.60 (2H, d), 7.02-7.04 (1H, d), 7.11 (1H, s), 7.17-7.18 (1H, d), 7.29-7.32 (2H, d), | 403 (M − 1) |
| | 7.48-7.50 (1H, d), 7.61-7.63 (1H, m), 7.72-7.88 (4H, m), 10-07-10-13 (1H, bs), | |
| | 11.64 (1H, s), 12.19-12.33 (1H, bs) | |
| 186 | 3.13 (2H, s), 6.67-6.69 (1H, d), 7.01-7.-03 (1H, d), 7.07-7.09 (1H, d), | 413 (M − 23)⁻ |
| | 7.22-7.24 (1H, s), 7.28 (1H, s), 7.34-7.56 (4H, m), 7.63-7.65 (2H, m), 7.69-7.73 (2H, t), | |
| | 7.81 (1H, s), 11.61 (1H, s) | |
| 187 | 2.26 (3H, s), 3.16 (2H, s), 6.67-6.69 (1H, d), 6.94-6.96 (2H, d), 7.08-7.13 (2H, d0, | 584 (M − 23)⁻ |
| | 7.16 (1H, s), 7.22-7.24 (1H, d), 7.32 (1H, s), 7.35-7.36 (1H, m), 7.38-7.40 (1H, m), | |
| | 7.44-7.48 (3H, m), 7.53-7.55 (2H, m), 7.57-7.63 (2H, m), 11.61 (1H, s) | |
| 188 | 1.31-1.33 (3H, t), 1.38-1.47 (3H, t), 3.19 (2H, s), 4.06-4.09 (2H, q), 4.13-4.17 (2H, q), | 499 (M + 1) |
| | 6.70-6.72 (1H, d), 7.01-7.05 (1H, m), 7.09-7.12 (1H, d), 7.24-7.27 (3H, t), 7.59 (1H, s), | |
| | 7.60-7.65 (1H, m), 7.70 (1H, s), 7.90 (1H, s), 11.50 (1H, bs) | |
| 189 | 3.13 (2H, s), 6.67-6.69 (1H, d), 7.01-7.-03 (1H, d), 7.08-7.10 (1H, d), 7.17 (1H, s) | 433 (M + 1) |
| | 7.21-7.24 (1H, m), 7.28-7.32 (2H, m), 7.51-7.53 (1H, d), 7.59-7.61 (1H, d), | |
| | 7.67-7.69 (2H, d), 7.70-7.74 (1H, m), 7.76-7.80 (1H, m), 11.61 (1H, s) | |
| 190 | 3.10-3.14 (4H, m), 3.17-3.18 (2H, s), 3.70-3.75 (4H, m), 6.65-6.67 (1H, d), | 424 (M + 1) |
| | 6.94-6.96 (1H, d), 6.98-7.00 (1H, d), 7.01-7.02 (1H, d), 7.03-7.09 (1H, t), 7.12 (1H, s), | |
| | 7.20-7.22 (1H, d), 7.29-7.31 (1H, d), 7.38-7.40 (1H, d), 11.65 (1H, s) | |
| 191 | 3.60 (2H, s), 7.03 (1H, bs), 7.29-7.31 (2H, m), 7.64-7.68 (2H, m), 7.71-7.75 (2H, m), | 457 (M − 1) |
| | 7.77-7.85 (1H, m), 7.87-7.91 (2H, m), 7.94-7.96 (2H, m), 8.01-8.06 (1H, m), | |
| | 11.60 (1H, s), 12.55-12.62 (2H, bs) | |
| 192 | 3.16 (2H, s), 6.68-6.70 (1H, d), 7.10-7.12 (1H, d), 7.21-7.29 (2H, m), | 419 (M − 23) |
| | 7.38-7.43 (3H, m), 7.65-7.70 (3H, m), 7.74-7.95 (1H, d), 8.32 (1H, s), 11.63 (1H, s) | |
| 193 | 3.36 (2H, s), 6.76-6.78 (1H, d), 7.12-7.14 (1H, d), 7.30-7.32 (1H, m), | 393 (M − 23) |
| | 7.40-7.43 (2H, m), 7.47-7.52 (2H, m), 7.57-7.61 (1H, d), 7.95-7.99 (1H, m), 8.03-8.06 (1H, m), | |
| | 11.63 (1H, s) | |
| 194 | 3.16-3.18 (2H, d), 3.81-3.84 (3H, d), 6.68-6.70 (1H, d), 6.90-6.93 (1H, m), | 443 (M − 23) |
| | 7.01-7.04 (1H, d), 7.18-7.20 (1H, d), 7.22-7.24 (2H, m), 7.29-7.31 (1H, m), 7.34-7.36 (1H, m), | |
| | 7.37-7.40 (1H, m), 7.51-7.53 (1H, d), 7.59-7.61 (1H, d), 7.69--771 (1H, d), | |
| | 7.66-7.78 (1H, d), 11.63 (1H, s) | |
| 195 | 1.32-1.37 (3H, t), 3.14-3.20 (2H, s), 4.03-4.10 (2H, q), 6.68-6.70 (1H, d), | 457 (M − 23) |
| | 6.98-7.04 (3H, q), 7.10-7.12 (1H, d), 7.16 (1H, d), 7.21-7.23 (1H, d), 7.47-7.49 (1H, d), | |
| | 7.56-7.60 (2H, t), 7.62-7.67 (2H, t), 7.70-7.72 (1H, d), 11.6 (1H, s) | |
| 196 | 3.14-3.18 (2H, s), 6.28-6.29 (2H, d), 6.69-6.71 (1H, d), 7.03--7.05 (1H, d), | 478 (M − 23) |
| | 7.10-7.12 (1H, d), 7.19 (1H, s), 7.22-7.25 (1H, m), 7.43-7.44 (1H, t), 7.46 (1H, t), | |
| | 7.53-7.55 (1H, d), 7.61-7.63 (1H, d), 7.66-7.70 (2H, t), 7.73-7.77 (3H, d), 7.81-7.85 (1H, t), | |
| | 11.60 (1H, s) | |
| 197 | 3.12-3.15 (2H, s), 5.32-5.35 (2H, d), 6.68-6.70 (1H, d), 7.02-7.04 (1H, d), | 511 (M − 23) |
| | 7.08-7.10 (1H, d), 7.21--7.23 (1H, d), 7.27-7.34 (3H, q), 7.21-7.24 (1H, d), 7.37-7.42 (1H, m), | |
| | 7.44-7.46 (1H, s), 7.48-7.49 (1H, s), 7.53-7.56 (1H, d), 7.63-7.68 (1H, m), | |
| | 7.75-7.78 (1H, t), 7.85 (1H, s), 11.64 (1H, s) | |
| 198 | 3.12-3.15 (2H, s), 4.52-4.54 (2H, s), 5.23 (1H, bs), 6.67-6.69 (1H, d), | 443 (M − Na) |
| | 7.01-7.03 (1H, d), 7.08--7.10 (1H, d), 7.17 (1H, s), 7.21-7.24 (1H, d), 7.37-7.40 (1H, m), | |
| | 7.42 (1H, s), 7.50-7.52 (1H, d), 7.59-7.63 (2H, m), 7.68-7.70 (2H, m), 7.75-7.77 (1H, d), | |
| | 11.64 (1H, s) | |
| 199 | 3.59-3.60 (2H, s), 6.84-6.89 (2H, q), 7.02-7.04 (1H, d), 7.30-7.34 (2H, t), 7.54--7.56 | 429 (M − 1) |
| | (2H, dd), 7.60-7.63 (1H, m), 7.66-7.70 (1H, d), 7.72-7.74 (3H, d), 7.78-7.80 (1H, d), | |
| | 11.64 (1H, s), 12.16-12.62 (1H, bs) | |
| 200 | 3.95 (2H, s), 7.22 (1H, m), 7.37-7.39 (4H, m), 7.40-7.50 (4H, m), 7.60-7.63 (2H, d), | 457 (M − 1) |
| | 7.76-7.78 (1H, m), 7.90-7.94 (1H, m), 11.77 (1H, bs), 12.54-12.57 (1H, bs), | |
| | 13.01-13.09 (1H, bs) | |
| 201 | 3.09-3.12 (2H, d), 4.04-4.08 (2H, d), 6.66-6.68 (1H, d), 6.82-6.87 (2H, m), | 487 (M − 1) |
| | 6.99-7.01 (1H, d), 7.07-7.09 (1H, d), 7.14 (1H, s), 7.21-7.23 (1H, m), 7.45-7.47 (2H, d), | |
| | 7.51-7.53 (1H, d), 7.56-7.61 (2H, m), 7.63-7.69 (1H, m), 11.65-11.68 (1H, s) | |
| 202 | 3.54 (2H, s), 5.03 (2H, s), 7.129-7.150 (2H, d), 7.506-7.526 (1H, d), | 401 (M − 1) |
| | 7.627-7.645 (2H, m), 7.818-7.839 (1H, d), 8.011-8.052 (2H, t), | |
| | 8.080-8.101 (1H, d), 8.304 (1H, s), 9.308 (1H, s), 11.6 (1H, s) 12.27 (2H, bs) | |
| 203 | 3.96 (2H, s), 3.98-4.01 (6H, d), 6.90-6.92 (1H, d), 7.03-7.05 (1H, d), | 447 (M − 1) |
| | 7.29-7.34 (3H, m), 7.39-7.50 (5H, m), 11.71 (1H, s), 12.60 (1H, bs) | |
| 204 | 1.68-1.76 (2H, m), 1.88-1.93 (2H, q), 2.45-2.50 (2H, bs), 6.73-6.75 (1H, d), | 441 (M − 23) |
| | 6.96-6.98 (1H, d), 7.04-7.06 (1H, d), 7.19 (1H, S), 7.25-7.27 (1H, d), 7.33-7.39 (1H, m), | |
| | 7.45-7.50 (2H, m), 7.51-7.53 (1H, t), 7.60-7.62 (1H, d), 7.66-7.70 (3H, t), 7.72-7.78 (1H, t), | |
| | 11.60 (1H, s) | |
| 205 | 2.10-2.11 (2H, q), 2.66-2.75 (2H, q), 6.72-6.74 (1H, d), 6.98-7.01 (1H, d), | 419 (M − 23) |
| | 7.07-7.09 (1H, d), 7.22-7.24 (1H, d), 7.30-7.34 (1H, d), 7.64-7.66 (1H, d), 7.69-7.74 (1H, m), | |
| | 7.87-7.90 (1H, d), 7.94-8.00 (2H, m), 8.03-8.06 (1H, m), 11.60 (1H, s) | |
| 206 | 3.14-3.16 (2H, s), 6.48-6.56 (1H, m), 7.31-7.39 (2H, m), 7.44-7.49 (3H, q), | 431 (M − 23) |
| | 7.53-7.55 (1H, d), 7.61-7.63 (1H, d), 7.66-7.68 (1H, d), 7.70-7.76 (4H, m), 11.60 (1H, s) | |
| 207 | 3.162 (2H, s), 3.797 (3H, s), 6.679-6.700 (1H, d)6.995-7.042 (2H, m), | 443 (M − 23) |
| | 7.064-7.139 (2H, m), 7.168 (1H, s), 7.221-7.241 (1H, m), 7.278-7.313 (1H, m), | |
| | 7.329-7.374 (1H, m), 7.449-7.505 (3H, q), 7.556-7.579 (1H, s), 11.6 (1H, s) | |
| 208 | 3.598-3.607 (2H, s), 6.956-6.978 (1H, m), 7.094-7.116 (1H, d), 7.306-7.353 (1H, t) | 487 (M + 1) |
| | 7.583-7.643 (2H, m), 7.676-7.686 (2H, m), 7.712-7.754 (1H, m), 7.776-7.801 (1H, m), | |
| | 7.827-7.879 (2H, m), 7.914-7.949 (2H, m), 7.975-7.997 (1H, m), 11.64 (1H, s) | |
| 209 | 3.152 (2H, s), 6.67-6.697 (1H, d), 7.021-7.042 (1H, d), | 449 (M − 23) |
| | 7.089-7.109 (1H, d), 7.181 (1H, s), 7.207-7.252 (2H, m), 7.438-7.455 (1H, d), | |
| | 7.529-7.550 (2H, d)7.611-7.632 (1H, d), 7.769-7.790 (1H, d), 7.844-7.865 (1H, d), 11.65 (1H.s) | |
| 210 | 3.60 (2H, s), 7.036 (1H, bs), 7.301-7.351 (1H, m), 7.425-7.447 (2H, d), | 492 (M − 1) |
| | 7.650-7.670 (1H, d), 7.713-7.728 (1H, d), 7.750-7.802 (2H, m), | |
| | 7.874-7.901 (2H, d), 7.926 (2H, s), 7.943-7.987 (3H, m), 11.65 (1H, s), 12.41 (1H, s) | |
| 211 | 2.15-2.22 (3H, s), 3.13-3.15 (2H, s), 6.19-6.22 (1H, d), 6.67-6.69 (1H, d), | 416 (M − 23) |
| | 6.91-6.97 (2H, d), 7.02-7.04 (1H, d), 7.08-7.10 (1H, d), 7.14 (1H, s), 7.20-7.26 (2H, m), | |
| | 7.30-7.39 (2H, m), 7.44-7.49 (1H, m), 11.50 (1H, s) | |
| 212 | 2.286-2.304 (3H, s), 3.171-3.179 (2H, s), 6.175-6.230 (2H, m), 6.673-6.694 (1H, d), | 582 (M − 1) |
| | 6.933-6.953 (1H, d), 7.016-7.036 (1H, d)7.099 (1H, s), 7.137-7.158 (2H, d), | |
| | 7.195-7.277 (3H, m), 7.319-7.339 (1H, d), 7.433-7.454 (2H, m), 7.486-7.534 (1H, m), | |
| | 7.581-7.615 (2H, t), 11.61 (1H, s) | |
| 214 | 1.323-1.373 (3H, t), 2.258-2.276 (3H, d), 3.173-3.185 (2H, s), 4.033-4.087 (2H, q), | 610 (M − 23) |
| | 6.676-6.697 (1H, d), 6.942 (1H, s), 6.963-6.981 (2H, d), 7.003-7.038 (2H, d), 7.131 (1Hs), | |
| | 7.150-7.182 (2H, d), 7.225-7.244 (1H, t), 7.452-7.473 (1H, d), 7.536-7.581 (2H, q), | |
| | 7.591-7.618 (2H, d), 7.639-7.657 (1H, t), 7.703-7.725 (1H, d), 11.61 (1H, s) | |
| 215 | 3H (3H, d), 3.109-3.191 (2H, t), 6.680-6.700 (1H, d)7.023-7.043 (1H, d), | 606 (M − 1)⁻ |
| | 7.131-7.178 (3H, t), 7.225-7.327 (3H, m), 7.509-7.547 (1H, t), 7.566-7.612 (3H, q), | |
| | 7.634-7.686 (2H, t), 7.700-7.800 (3H, m), 11.65 (1H, s) | |
| 216 | 3.128-3.189 (2H, d), 3.794-3.815 (3H, d), 6.695-6.675 (1H, d), | 461 (M − 23) |
| | 7.004-7.053 (2H, dd), 7.080-7.109 (1H, d), | |
| | 7.214-7.234 (1H, d), 7.296 (1H, s), 7.36 (1H, s), 7.492, (1H, s), 7.525-7.575 (1H, q), | |
| | 7.649-7.670 (2H, d), 7.715-7.743 (1H, t), 11.65 (1H, bs) | |
| 217 | 3.141-3.161 (2H, d), 5.242-5.309 (2H, d), 6.698-6.718 (1H, d), 7.035-7.055 (1H, d), | 512 (M − 23) |
| | 7.112-7.132 (1H, d), 7.212-7.276 (3H, m), 7.314-7.342 (4H, t), | |
| | 7.370-7.432 (2H, m), 7.494 (1H, s), 9.779-9.806 (1H, s), 11.6 (1H, bs) | |
| 218 | 3.738-3.802 (3H, s), 6.281-6.341 (1H, t), 6.930-6.948 (2H, d), 7.198-7.280 (1H, m) | 479 (M − 1)⁻ |
| | 7.365-7.483 (1H, m), 7.600 (2H, s), 7.688-7.705 (3H, d), 7.794-7.824 (3H, m), | |
| | 11.65-11.68 (1H, s), 12.56 (1H, s) | |
| 219 | 3.346 (2H, s), 6.731-6.750 (2H, d), 7.047-7.065 (1H, d), | 620 (M − 23) |
| | 7.115-7.133 (2H, d), 7.202 (1H, s), 7.274-7.391 (3H, m), 7.434-7.540 (4H, m), | |
| | 7.608-7.783 (5H, m), 11.63 (1H, s) | |
| 220 | 2.539 (3H, s), 3.121 (2H, s), 6.664-6.684 (1H, d), 7.022-7.022 (1H, d), | 459 (M − 23) |
| | 7.070-7.090 (1H, d), 7.163 (1H, s), 7.213-7.235 (1H, d)7.318-7.366 (2H, m), | |
| | 7.499-7.519 (1H, d), 7.585-7.617 (2H, m), 7.638-7.700 (2H, d), 7.746-7.766 (1H, d), 11.63 (1H, s) | |
| 221 | 3.140 (2H, s), 6.670-6.690 (1H, d), 7.016-7.037 (1H, d), 7.081-7.101 (1H, d), | 449 (M − 23) |
| | 7.165-7.245 (3H, m), 7.537-7.575 (3H, d), | |
| | 7.592-7.616 (1H, m), 7.641 (1H, s), 7.664 (1H, bs), 11.63 (1H, s) | |
| 222 | 3.12-3.14 (2H, d), 6.28-6.29 (2H, s), 6.96-7.15 (2H, m), 7.21-7.29 (1H, d), | 496.24 (M − 23) |
| | 7.44-7.46 (2H, t), 7.54-7.56 (1H, d), 7.61-7.70 (3H, m), 7.76-7.81 (3H, d), | |
| | 7.81-7.89 (2H, t), 11.60 (1H, s) | |
| 223 | 3.338 (2H, s), 6.695-6.715 (1H, d), 6.928-6.938 (1H, t), 6.949-6.990 (1H, d), | 558 (M − 23) |
| | 7.046-7.066 (1H, d), 7.188 (1H, s), 7.238-7.256 (1H, d)7.330-7.382 (2H, m), | |
| | 7.434-7.472 (2H, m), 7.519-7.539 (2H, m), 7.605-7.625 (1H, d), 7.625-7.688 (3H, m), | |
| | 7.746-7.780 (1H, m), 11.63 (1H, s) | |
| 224 | 3.164 (2H, s), 6.686-6.706 (1H, d), 6.942-6.962 (1H, d), 7.019-7.039 (1H, d), | 570 (M − 23) |
| | 7.142-7.186 (2H, m), 7.226-7.254 (1H, m), 7.328-7.365 (1H, m), 7.434-7.471 (2H, m), | |
| | 7.500-7.536 (2H, m), 7.604-7.624 (1H, d), 7.624-7.679 (3H, m), | |
| | 7.727-7.780 (3H, m), 11.63 (1H, s) | |
| 225 | 2.682-2.715 (3H, d), 3.164-3.214 (2H, d), 6.714-6.734 (1H, d), 7.021-7.041 (1H, d), | 490 (M − 23) |
| | 7.094-7.115 (1H, d), 7.186 (1H, s), 7.254-7.266 (1H, m), 7.326-7.363 (1H, m), | |
| | 7.432-7.477 (2H, m), 7.500-7.541 (2H, m), 7.606-7.626 (1H, d), 7.660-7.713 (2H, m), | |
| | 7.741-7.70 (1H, m), 11.62 (1H, s) | |
| 226 | 3.13-3.17 (2H, d), 3.73-3.76 (3H, d), 6.70-6.72 (1H, d), 6.86-6.88 (2H, d), | 582 (M − 23) |
| | 6.99 (1H, bs), 7.03-7.05 (1H, d), 7.21 (1H, bs), 7.28-7.34 (1H, m), 7.34-7.39 (1H, m), | |
| | 7.43-7.47 (2H, m), 7.50-7.54 (2H, m), 7.61-7.67 (4H, m), 7.68-7.79 (2H, m), 11.63 (1H, s) | |
| 227 | 3.12-3.15 (2H, s), 6.67-6.69 (1H, d), 7.02-7.04 (1H, d), 7.08-7.10 (1H, d), | 497 (M − 23) |
| | 7.18 (1H, s), 7.21-7.25 (1H, t), 7.42-7.47 (2H, t), 7.53-7.55 (1H, d), 7.61-7.63 (1H, d), | |
| | 7.70-7.72 (1H, d), 7.77-7.79 (2H, d), 7.85-7.87 (1H, d), 11.65-11.68 (1H, s) | |
| 228 | 3.50 (2H, s), 6.86-6.87 (2H, m), 7.01-7.21 (1H, m), 7.30-7.34 (2H, m), | 447 (M − 1) |
| | 7.49-7.52 (2H, m), 7.61-7.80 (5H, m), 9.80 (1H, s), 11.63 (1H, s), 12.39 (1H, s) | |
| 229 | 3.12-3.15 (2H, d), 3.87-3.89 (3H, d), 6.67-6.69 (1H, d), 6.97-7.01 (1H, d), | 461 (M − 23) |
| | 7.08-7.10 (1H, d), 7.16 (1H, s), 7.21-7.28 (2H, t), 7.47-7.50 (2H, t), 7.54-7.59 (1H, t), | |
| | 7.67-7.69 (2H, t), 7.74-7.76 (1H, d), 8.54 (1H, s) | |
| 230 | 2.06-2.07 (3H, s), 3.13-3.16 (2H, d), 6.68-6.70 (1H, d), 7.02-7.04 (1H, d), | 470.10 (M − 23) |
| | 7.09-7.11 (1H, d), 7.17 (1H, s), 7.22-7.23 (1H, d), 7.49-7.51 (1H, d), 7.57-7.71 (6H, m), | |
| | 7.73-7.75 (1H, d), 10.06-10.08 (1H, s), 11.58 (1H, s) | |
| 231 | 3.13-3.19 (2H, s), 6.68-6.70 (1H, d), 7.02-7.04 (1H, d), 7.09-7.11 (1H, d), | 481 (M − 23) |
| | 7.19-7.22 (1H, s), 7.24-7.26 (1H, t), 7.56-7.58 (1H, d), 7.64-7.66 (1H, d), 7.77-7.79 (3H, t), | |
| | 7.81-7.84 (1H, s), 7.86-7.90 (1H, d), 7.96-7.98 (1H, d), 11.61 (1H, s) | |
| 232 | 1.03-1.09 (2H, m), 1.15-1.27 (3H, m), 1.53-1.80 (6H, m), 3.11-3.19 (2H, s), | 525 (M − 23) |
| | 3.80-3.84 (2H, t), 6.67-6.69 (1H, d), 6.84 (1H, m), 6.98-7.00 (1H, d), 7.02-7.04 (1H, m), | |
| | 7.089-7.11 (1H, d), 7.16 (1H, s), 7.21-7.22 (1H, m), 7.47-7.49 (1H, d), 7.54-7.57 (2H, d), | |
| | 7.62-7.64 (1H, d), 7.69-7.75 (1H, m), 7.79-7.81 (1H, m), 11.61 (1H, s) | |
| 233 | 3.19-3.21 (2H, s), 6.27 (1H, m), 6.64-6.69 (1H, d), 6.99-7.03 (2H, m), | 447 (M − 1⁾⁻ |
| | 7.09-7.15 (3H, m), 7.21-7.23 (1H, d), 7.31-7.33 (1H, d), 7.39-7.45 (2H, m), 7.48-7.52 (1H, m), | |
| | 11.60 (1H, s) | |
| 234 | 2.94-2.96 (3H, d), 3.13-3.16 (2H, d), 3.33-3.35 (3H, d), 6.68-6.70 (1H, d), | 456 (M − 23) |
| | 6.78-6.83 (2H, t), 7.02-7.04 (1H, d), 7.09-7.11 (1H, d), 7.15 (1H, s), 7.21-7.24 (1H, d), | |
| | 7.44-7.46 (1H, d), 7.52-7.54 (2H, d), 7.59-7.62 (2H, t), 7.68-7.70 (1H, d), 11.61 (1H, s) | |
| 235 | 2.34 (3H, s), 3.23-3.25 (2H, s), 6.77-6.77 (1H, d), 7.09-7.11 (1H, d), 7.17-7.19 (1H, s), | 666 (M − 23) |
| | 7.24-7.29 (3H, m), 7.44-7.46 (2H, t), 7.55-7.59 (4H, t), 7.65-7.72 (3H, q), 7.81-7.83 (1H, d), | |
| | 8.31 (1H, s), 11.63 (1H, s) | |
| 236 | 3.14-3.17 (2H, d), 3.87-3.89 (3H, s), 6.49-6.57 (1H, m), 6.97-7.07 (1H, m), | 435 (M − 23) |
| | 7.12-7.20 (1H, m), 7.27-7.39 (2H, m), 7.57-7.65 (2H, m), 7.75-7.80 (1H, d), 7.96 (1H, s), | |
| | 8.53 (1H, s), 11.62 (1H, s) | |
| 237 | 3.60 (2H, s), 7.30-7.35 (1H, s), 7.69-7.75 (1H, d), 7.78-7.86 (2H, d), 8.13-8.15 (2H, d), | 416 (M + 1) |
| | 8.24-8.26 (2H, d), 8.89-8.90 (3H, d), 9.35 (1H, S), 9.57 (1H, s), 11.65 (1H, s), | |
| | 12.03 (1H, bs) | |
| 238 | 2.53 (3H, s), 3.16 (2H, s), 6.68-6.70 (1H, d), 7.03-7.05 (1H, d), 7.10-7.12 (1H, d), | 477 (M − 23) |
| | 7.21-7.23 (1H, d), 7.30-7.37 (3H, m), 7.48-7.54 (2H, m), 7.61-7.65 (2H, d), | |
| | 7.73-7.75 (1H, d), 11.63 (1H, s) | |
| 239 | 2.51 (3H, s), 3.20 (2H, s), 6.99-7.01 (1H, d), 7.19-7.25 (1H, m), 7.31-7.39 (2H, m), | 684 (M − 23) |
| | 7.49-7.53 (1H, m), 7.68-7.70 (4H, m), 7.72-7.78 (3H, m), | |
| | 7.89-7.92 (2H, m), 8.12 (2H, m), 11.63 (1H, s) | |
| 240 | 3.18 (2H, s), 5.22-5.24 (2H, s), 6.69-6.71 (1H, d), 7.03-7.05 (1H, d), 7.10-7.12 (1H, d), | 493 (M − 23) |
| | 7.19-7.21 (1H, d), 7.23-7.25 (1H, d), 7.28-7.30 (1H, s), 7.32-7.34 (2H, d), | |
| | 7.36-7.44 (3H, d), 7.50-7.54 (1H, d), 7.56-7.58 (1H, d), 7.76-7.89 (3H, m), 11.60 (1H, s) | |
| 241 | 1.07-1.27 (3H, t), 1.71-1.73 (3H, d), 2.21-2.23 (3H, s), 4.20-4.24 (2H, q), | 515 (M − 1) |
| | 6.84-6.99 (1H, m), 7.30-7.34 (1H, t), 7.38-7.50 (3H, t), 7.62-7.64 (1H, d), 7.73-7.83 (5H, m), | |
| | 7.82-7.83 (2H, d), 11.89 (1H, s) | |
| 242 | 2.11-2.19 (3H, s), 3.13-3.20 (2H, s), 6.20-6.25 (2H, m), 6.66-6.68 (1H, d), | 416 (M − 23) |
| | 6.89-6.92 (1H, t), 6.98-7.04 (1H, m), 7.07-7.10 (2H, d), 7.15-7.22 (1H, m), 7.28 (1H, s), | |
| | 7.34-7.43 (2H, m), 7.44-7.47 (1H, m), 11.62 (1H, s) | |
| 243 | 3.63-3.71 (2H, s), 7.01-7.029 (1H, d), 7.29-7.33 (2H, t), 7.64-7.68 (3H, t), | 438 (M− 1) |
| | 7.75-7.77 (1H, d), 7.84-8.02 (6H, m), 11.9 (1H, bs), 12.32 (1H, s) | |
| 244 | 2.67-2.72 (2H, s), 3.44-3.56 (3H, d), 6.83-6.87 (1H, m), 7.05-7.11 (1H, m), | 427 (M − 1) |
| | 7.17-7.19 (1H, d), 7.27-7.33 (2H, d), 7.39 (1H, m), 7.44-7.51 (3H, q), 7.63-7.96 (4H, m), | |
| | 11.65 (1H, s), 12.07-12.33 (1H, bs) | |
| 245 | 2.34-2.39 (3H, s) 3.54 (2H, s) 6.79 (1H, s) 7.20-7.26 (2H, d) 7.39-7.43 (4H, m), | 550 (M − 1) |
| | 7.49-7.53 (2H, t), 7.61-7.63 (2H, d) 7.77-7.82 (6H, m) 7.96-7.98 (1H, d), 11.70 (1H, s), | |
| | 12.31-12.37 (1H, s) | |
| 246 | 0.92-0.95 (6H, d), 3.82-3.89 (2H, s), 3.91 (3H, s), 4.07-4.14 (1H, m), | 501 (M − 1) |
| | 7.01-7.05 (1H, s), 7.22 (1H, s), 7.26-7.28 (1H, s), 7.31-7.35 (2H, d), 7.38-7.39 (2H, d), | |
| | 7.41-7.45 (2H, d), 7.47-7.49 (2H, t), 7.76 (1H, s), 11.55 (1H, s), 12.37-12.39 (1H, s) | |
| 247 | 3.50-3.67 (2H, s), 3.79-4.04 (3H, d), 6.28-6.31 (1H, d), 6.83-6.90 (1H, s), | 466 (M − 1) |
| | 7.01-7.09 (1H, d), 7.13-7.20 (2H, q), 7.29-7.39 (2H, q), 7.43-7.49 (1H, s), 7.63-7.71 (1H, s), | |
| | 7.73-7.79 (1H, s), 7.83 (1H, s), 11.62 (1H, s), 12.36-12.55 (1H, s) | |
| 248 | 1.15-1.26 (3H, t), 4.11-4.24 (2H, q), 5.23 (1H, s), 6.35-6.39 (1H, t), 6.66-6.68 (1H, d), | 565 (M − 1) |
| | 6.91-6.96 (1H, m), 7.01-7.15 (1H, m), 7.37-7.39 (2H, t), 7.44-7.51 (5H, t), | |
| | 7.56-7.65 (4H, q), 7.68-7.79 (2H, m), 11.72 (1H, s) | |
| 249 | 2.33 (3H, s), 3.15 (3H, s), 3.45 (2H, s), 6.51-6.53 (1H, d), 6.70-6.72 (1H, dd), | 580 (M − 1) |
| | 6.80-6.82 (1H, d), 7.01-7.06 (1H, m), 7.13-7.16 (1H, m), 7.17-7.20 (1H, m), 7.29-7.36 (4H, m), | |
| | 7.39-7.43 (2H, m), 7.53-7.62 (2H, m), 7.69-7.71 (3H, m), 11.63 (1H, s), 12.29 (1H, s) | |
| 250 | 1.25-1.34 (3H, t), 3.43-3.70 (2H, q), 7.38-7.42 (2H, m), 7.44-7.53 (4H, m), | 470 (M − 1) |
| | 7.62-7.65 (2H, d), 7.66-7.72 (2H, d), 7.75-7.77 (3H, d), 7.86-7.90 (1H, d), 11.94 (1H, s), | |
| | 12.52-12.66 (1H, s) | |
| 251 | 0.89-0.95 (6H, d), 2.38 (3H, s), 3.52 (2H, s), 3.81-3.91 (3H, d), | 654 (M − 1) |
| | 4.11-4.13 (1H, m), 6.97-7.05 (1H, d), 7.15-7.25 (3H, m), | |
| | 7.37-7.43 (4H, d), 7.45-7.47 (2H, m), 7.54-7.56 (2H, d), 7.68 (2H, s), | |
| | 7.76-7.77 (2H, d), 11.54 (H, s), 12.28-12.30 (1H, s) | |
| 252 | 3.14-3.17 (2H, s), 3.83 (3H, s), 6.67-6.69 (1H, d), | 436 (M + 1)⁺ |
| | 6.89-6.94 (1H, d), 6.96-6.99 (1H, d), 6.03-6.08 (2H, d), 6.10-6.14 (2H, d), | |
| | 7.19-7.21 (1H, d), 7.77-7.80 (1H, d), 7.84-7.86 (1H, d), | |
| | 11.63 (1H, s) | |
| 253 | 3.29 (2H, s), 3.60 (2H, s), 5.19 (1H, q), 7.23-7.25 (3H, m), 7.30-7.33 (1H, m), | 417 (M − 1) |
| | 7.38-7.42 (1H, m), 7.46-7.49 (4H, m), 7.52-7.54 (3H, m), 11.08 (1H, s), 12.2 (1H, s) | |
| 254 | 2.29-2.3 (3H, d), 3.13-3.15 (2H, s), 3.42 (3H, s), 6.17-6.29 (2H, s) | 622 (M + 1) |
| | 6.48 (1H, s), 6.80 (1H, s), 6.91-6.97 (1H, d), 7.06-7.12 (2H, t), | |
| | 7.19-7.24 (1H, s), 7.30-7.44 (2H, d), 7.54-7.60 (1H, s), | |
| | 7.62-7.69 (2H, s), 7.81 (1H, s), 8.07 (1H, s), 12.20-12.60 (2H, s), | |
| 255 | 3.29-3.41 (2H, s), 3.87 (3H, s), 6.95 (1H, s), 7.25 (4H, s), | 479 (M − 1) |
| | 7.43 (2H, s), 7.61 (1H, s), 7.74-7.95 (3H, t), 11.60 (1H, s), | |
| | 11.95-12.15 (1H, s) | |
| 256 | 7.27 (1H, s), 7.40-7.42 (1H, d), 7.47-7.51 (3H, q), 7.62-7.64 (1H, d), 7.67-7.70 (2H, d), | 427 (M − 1) |
| | 7.72-7.77 (2H, m), 7.80 (2H, m), 7.86-7.88 (1H, d), 7.98-8.00 (1H, m), 12.04 (1H, s), | |
| | 12.68 (1H, s) | |
| 257 | 1.06-1.24 (5H, m), 1.37-1.43 (1H, m), 1.55-1.60 (2, m), 6.59-6.60 (1H, d), | 485 (M − 1) |
| | 7.09-7.13 (2H, s), 7.32-7.40 (3H, dd), 7.43-7.48 (4H, m), 7.49-7.51 (2H, d), 7.55-7.64 (1H, d), | |
| | 11.60 (1H, s) | |
| 258 | 2.33-2.38 (3H, s), 3.52-3.57 (2H, s), 3.94 (3H, s), 7.04 (1H, s), | 612 (M − 1) |
| | 7.20 (2H, s), 7.26-7.30 (3H, d), 7.32-7.37 (3H, d), | |
| | 7.39-7.44 (3H, t), 7.57-7.59 (1H, d), 7.64-7.66 (1H, t), 7.68-7.73 (1H, d), | |
| | 7.75-7.77 (1H, d), 9.50-9.53 (1H, d), 11.49 (1H, s), 12.31 (1H, s) | |
| 259 | 2.15-2.25 (3H, s), 2.30 (3H, s), 3.14-3.23 (2H, s), 6.20-6.25 (2H, td), 6.66-6.68 (1H, d), | 569 (M − [symbol] |
| | 6.91-6.92 (1H, t), 6.96-6.98 (1H, d), 6.99-7.02 (2H, m), | |
| | 7.11-7.16 (2H, d), 7.21 (1H, s), 7.28 (1H, s), 7.33-7.41 (2H, m), 7.43-7.47 (1H, m), | |
| | 7.58-7.62 (2H, d), 11.60 (1H, s) | |
| 260 | 3.55-3.59 (2H, s), 3.91-3.97 (3H, s), 6.86-6.95 (1H, s), | 497 (M [symbol] |
| | 7.18-7.29 (3H, m), 7.42-7.45 (2H, q), 7.64-7.74 (1H, d), | |
| | 7.80-7.89 (2H, m)7.96-7.99 (1H, s), 11.74 (1H, s), | |
| | 12.49-12.62 (1H, s) | |
| 261 | 3.55-3.64 (2H, s), 6.86-6.90 (1H, s)7.36-7.43 (1H, d) | 503 (M [symbol] |
| | 7.57-7.66 (4H, m), 7.75-7.85 (3H, m), 11.9 (1H, s), | |
| | 12.68-12.71 (1H, s) | |
| 262 | 1.32-1.37 (3H, t), 2.42-2.44 (2H, m) 3.14-3.20 (2H, s), 4.03-4.10 (2H, q), | |
| | 6.68-6.70 (1H, d), 6.98-7.04 (3H, q), 7.10-7.12 (1H, d), 7.16 (1H, d), 7.21-7.23 (1H, d), | |
| | 7.47-7.49 (1H, d), 7.56-7.60 (2H, t), 7.62-7.67 (2H, t), 7.70-7.72 (1H, d), 11.6 (1H, s) | |
| 263 | 0.33-0.58 (4H, d), 1.14-1.23 (1H, m), 3.55-3.57 (2H, s), | 483 (M [symbol] |
| | 3.84-4.03 (2H, d), 6.99-7.04 (2H, m), 7.26-7.28 (2H, d), | |
| | 7.54-7.56 (2H, d), 7.62-7.70 (4H, m), 7.72-7.85 (2H, m), 7.88-7.92 (1H, m), 11.60 (1H, s), 12.30 (1H, s) | |
| 264 | 3.57-3.58 (2H, s), 4.82-4.86 (2H, s), 6.93-6.95 (1H, d), | 696 (M [symbol] |
| | 7.26-7.31 (2H, m), 7.397.46 (4H, m), 7.507.58 (3H, m), 7.637.67 (2H, d), 11.62 (1H, s), | |
| | 11.95-12.46 (1H, bs) | |
| 265 | 3.54 (2H, s), 3.73 (3H, s), 3.91-3.95 (3H, s), 6.99 (1H, s), | 475 (M [symbol] |
| | 7.09 (2H, s), 7.26 (3H, s), 7.46 (1H, s), 7.53-7.61 (3H, t) | 503 (M [symbol] |
| | 7.68-7.74 (1H, s), 7.89 (1H, s), 11.6 (1H, s), 12.06-12.41 (1H, s) | |

[symbol]

indicates data missing or illegible when filed

### Method of Inhibiting PTP 1B Enzyme

In another embodiment of the present invention there is provided a method of inhibiting PTP1B enzyme, by an effective amount of one or more of compound, represented by the formula (I), or their pharmaceutically acceptable salts.

The compounds of the present invention possess the ability to inhibit an enzyme PTP1B and thereby useful in the treatment of diseases such as mediated by an enzyme PTP1B

Inhibitory activity of compounds for this application is demonstrated using the following PTP1B enzyme inhibition assay.

### Principle:

Protein tyrosine phosphatase 1B (PTP1B) is an intracellular enzyme which functions by removing the phosphate groups from phosphorylated tyrosine residues of cellular proteins. PTP1B has been implicated as a negative regulator of insulin action and therefore is of therapeutic interest. The ability of the test compounds to inhibit the activity of PTP1B is determined using an enzyme inhibition assay.

The assay is performed using two independent substrates of PTP1B namely, p-nitrophenyl phosphate (pNpp) and the phosphorylated Epidermal growth factor receptor peptide (EGFR).

In the assay performed with pNpp, the action of PTP1B results in the release of p-nitrophenol, which is yellow in colour and can be measured at 410 nm. In the case of the assay using EGFR, action of PTP1B results in the release of free phosphate group. This is trapped using a reagent containing ammonium molybdate and malachite green resulting in a colored complex whose absorbance can be measured at 620 nm.

### Materials:

Dimethyl sulfoxide (DMSO) (Sigma)

Dithiothreitol (DTT) (Gibco)

Ethylenediamine tetra acetic acid (EDTA) (Gibco)

Igepal® CA-630 (Sigma)

1M N-(2-Hydroxyethyl)piperazine-N′-(2-ethanesulfonicacid); 4-(2-Hydroxyethyl)piperazine-1-ethanesulfonic acid (HEPES) (Hyclone) p-Nitrophenyl phosphate (pNp) (SRL

Recombinant human Protein-tyrosine phosphatase1B (PTP1B) (amino acid 1-322 Biomol, USA)

### Stock Solutions:

1M HFPES (Store at 4° C.)

10×EDTA 20 mM in 100 mM HEPES (Store aliquot at −20° C.)

10×DTT 20 mM in 100 mM IEPES (Store aliquot at −20° C.)

10% Igepal® CA-630 in 100 mM HEPES (Store at 4° C.)

### Prepare Fresh Daily:

100 mM HEPES

1% Igepal® CA-630

1× Assay buffer 50 mM HEPES (pH 7.4)
1 mM EDTA
1 mM DTT
0.05% Igepal® CA-630

### Enzyme Dilution:

Dilute recombinant Human PTP1B 1:150 in 1× Assay buffer for 10× stock,

### Substrate Preparation:

Prepare 10× stock of pNpp (50 mM) in 1× Assay buffer

### Compound Preparation:

Prepare 100× stock in 100% DMSO, dilute 1:10 with 1× Assay buffer to get 10× stock in 10% DMSO.

### Procedure:

PTP1B inhibition assay was performed using recombinant human PTP1B. PTP1B (0.016 μg/well) was incubated in a buffer containing 50 mM HEPES, 1 mM DTT, 1 mM EDTA and 0.05% Igepal® CA-630 with or without inhibitor (10× stock, final DMSO concentration is 1%) for 30 mins at 25° C. This was followed by addition of p-Nitrophenyl phosphate (pNpp) substrate (5 mM). The final volume of the reaction mixture is 50 μL and experiment was done in 96 well half area plate. The incubation was continued for 30 mins at 25° C. The conversion of pNpp to p-Nitrophenol (pNp) was measured at 410 nm in Spectramaxplus Spectrophotometer (Molecular Devices, USA).

$$\% Inhibition was expressed as$$

$$\% Inhibition = \frac{OD ( control ) - OD ( test )}{OD ( control )} \times 100$$

The representative compounds according to the present invention are exemplified hereinafter.

A good number of them have been subjected to in-vitro test according to the above procedure, the results of which are given below in Table 2:

TABLE 2
(In Vitro results):

| Comp. | |
|---|---|
| NO | Activity Score |
| 1 | (+++) |
| 2 | (+++) |
| 3 | (+++) |
| 5 | (+) |
| 7 | (+++) |
| 8 | (+) |
| 9 | (+++) |
| 10 | (++) |
| 11 | (++) |
| 13 | (++) |
| 14 | (++) |
| 16 | (+) |
| 17 | (++) |
| 18 | (+++) |
| 19 | (+++) |
| 21 | (+++) |
| 22 | (+++) |
| 23 | (+++) |
| 26 | (+) |
| 28 | (+++) |
| 32 | (+++) |
| 33 | (+) |
| 34 | (+++) |
| 36 | (+) |
| 37 | (+++) |
| 43 | (+++) |
| 44 | (+) |
| 45 | (+++) |
| 47 | (+++) |
| 48 | (+++) |
| 49 | (++) |
| 50 | (+++) |
| 51 | (+++) |
| 53 | (++) |
| 54 | (+++) |
| 55 | (++) |
| 56 | (+) |
| 59 | (+++) |
| 60 | (++) |
| 61 | (+) |
| 62 | (+++) |
| 67 | (++) |
| 69 | (+++) |
| 71 | (+++) |
| 72 | (+) |
| 78 | (+++) |
| 79 | (+++) |
| 80 | (+++) |
| 81 | (+++) |
| 82 | (+++) |
| 83 | (+++) |
| 84 | (+++) |
| 85 | (+++) |
| 87 | (+) |
| 88 | (+++) |
| 89 | (+) |
| 90 | (+++) |
| 91 | (++) |
| 92 | (++) |
| 94 | (+) |
| 95 | (+) |
| 96 | (+++) |
| 97 | (+++) |
| 99 | (+++) |
| 100 | (++) |
| 102 | (+++) |
| 103 | (+) |
| 104 | (++) |
| 113 | (+++) |
| 116 | (+++) |
| 117 | (+++) |
| 119 | (+++) |
| 120 | (+++) |
| 122 | (+++) |
| 123 | (+++) |
| 125 | (+++) |
| 126 | (++) |
| 128 | (+++) |
| 129 | (+++) |
| 130 | (++) |
| 131 | (+++) |
| 133 | (+++) |
| 134 | (++) |
| 142 | (++) |
| 143 | (+++) |
| 144 | (++) |
| 146 | (+++) |
| 152 | (+++) |
| 158 | (+++) |
| 159 | (+++) |
| 160 | (+) |
| 163 | (+++) |
| 164 | (++) |
| 165 | (+++) |
| 166 | (+++) |
| 169 | (+++) |
| 170 | (+++) |
| 172 | (+++) |
| 173 | (+++) |
| 174 | (+++) |
| 175 | (+++) |
| 177 | (+++) |
| 178 | (+++) |
| 179 | (+++) |
| 181 | (+) |
| 189 | (+++) |
| 191 | (+++) |
| 194 | (+++) |
| 195 | (+++) |
| 196 | (+++) |
| 198 | (+++) |
| 199 | (+++) |
| 200 | (+++) |
| 201 | (+++) |
| 205 | (+++) |
| 206 | (+++) |
| 207 | (+++) |
| 209 | (+++) |
| 210 | (+++) |
| 212 | (++) |
| 213 | (++) |
| 214 | (+++) |
| 215 | (+++) |
| 216 | (+++) |
| 217 | (+) |
| 219 | (+++) |
| 220 | (+++) |
| 221 | (+++) |
| 222 | (+++) |
| 223 | (++) |
| 224 | (++) |
| 225 | (+) |
| 226 | (+++) |
| 227 | (+) |
| 228 | (+++) |
| 229 | (+++) |
| 230 | (+++) |
| 231 | (+) |
| 232 | (+++) |
| 233 | (+++) |
| 234 | (+++) |
| 235 | (+++) |
| 238 | (+++) |
| 239 | (+) |
| 242 | (++) |
| 243 | (+++) |

Scoring Guide: +++ = >50% inhibition, ++ = 20-50% inhibition, + = 10-20% inhibition

According to another embodiment, the invention is to provide a method of prevention or treatment of disease conditions caused by overexpressed or altered by the PTP 1B enzyme through administration to a patient in need an therapeutically effective amount of compound of formula (I) or their pharmaceutically acceptable salts or pharmaceutical composition of compound of general formula (I) or their pharmaceutically acceptable salts.

According to another embodiment, the present invention provides a method of treating or delaying the onset and progression of diabetes, comprising administering a therapeutically effective amount of a compound of formula (I) or their pharmaceutically acceptable salts.

According to another embodiment, the present invention provides a method of treating impaired glucose tolerance and insulin resistance, comprising administering a therapeutically effective amount of a compound of formula (I) or their pharmaceutically acceptable salts.

According to another embodiment, the present invention provides a method of treating obesity comprising administering a therapeutically effective amount of a compound of formula (I) or their pharmaceutically acceptable salts.

Yet another embodiment of the present invention is to provide a method of treatment of autoimmune disorders, acute and chronic inflammatory disorders, osteoporosis, cancer, malignant disorders comprising administering a therapeutically effective amount of a compound of formula (I) or their pharmaceutically acceptable salts.

Yet another embodiment of the invention is to use compound of general formula (I) or their pharmaceutically acceptable salts in the manufacture of medicaments useful for prevention or treatment of disease conditions in a mammal mediated by overexpressed or altered PTP 1B enzyme.

Yet another embodiment of the invention is to use compound of general formula (I) or their pharmaceutically acceptable salts in the manufacture of medicaments useful for treatment or delaying the onset or progression of diabetes.

Yet another embodiment of the invention is to use compound of general formula (I) or their pharmaceutically acceptable salts in the manufacture of medicaments useful for treatment of impaired glucose tolerance and insulin resistance.

Yet another embodiment of the invention is to use compound of general formula (1) or their pharmaceutically acceptable salts in the manufacture of medicaments useful for treatment of obesity.

Yet another embodiment of the invention is to use compound of general formula (I) or their pharmaceutically acceptable salts in the manufacture of medicaments useful for treatment of autoimmune disorders, acute and chronic inflammatory disorders, osteoporosis, cancer, malignant disorders.

In a further embodiment of the invention, in addition to the treatment of disorders mediated by PTP 1B, the compound of formula (I) or their pharmaceutically acceptable salts are also useful for the development and standardization of in vitro and in vivo test systems for the evaluation of the effects of inhibitors of PTP 1B for the search of a new therapeutic agents.

In further embodiment, the invention encompasses pharmaceutical compositions for treating PTP-1B mediated diseases as defined above comprising a therapeutically effective amount of the active compound of general formula (I) and one or more other pharmaceutically active compounds, such as anti-diabetic compounds, anti-obesity compounds, and compounds that improve the lipid profile of the patient.

Thus, the methods of treatment or prevention described herein may further be comprised of administering to said patient a second anti-diabetic compound in an amount effective to treat, control, or prevent diabetes with the PTP-1B inhibitors of this invention.

Similarly, the methods of treatment or prevention described herein may further be comprised of administering to said patient a second anti-obesity compound in an amount effective to treat, control, or prevent obesity with the PTP-1B inhibitors of this invention.

Similarly, the methods of treatment or prevention described herein may further be comprised of administering to said patient a cholesterol lowering compounds, in an amount effective to improve the lipid profile in combination with a PTP-1B inhibitor of this invention. This may be beneficial in treating or preventing atherosclerosis and other metabolic conditions that often are associated with Type 2 diabetes.

Examples of other pharmaceutically active compounds that may be combined with a compound of Formula (I) and administered in combination with the PTP-1B inhibitors comprises of the following therapeutic classes but not limited to antidiabetics including insulin sensitizers such as PPAR.gamma. agonists; biguanides; insulin or insulin mimetics; sulfonylureas; alpha-glucosidase inhibitors; PPAR.alpha/gamma dual.agonists; glucokinase activators; glycogen phosphorylase inhibitors; AGE breakers; AGE inhibitors; meglitinides; SGLT2 inhibitors and the like, cholesterol lowering agents such as HMG-CoA reductase inhibitors; sequestrants; nicotinyl alcohol, nicotinic acid or a salt thereof; fibrates; inhibitors of cholesterol absorption for example beta-sitosterol; acyl CoA:cholesterol acyltransferase inhibitors; and the like, antiobesity including appetite suppressants; neuropeptide Y5 inhibitors; leptin, which is a peptidic hormone; beta3 adrenergic receptor agonists; cannabinoid receptor antagonists; PPAR.gamma. antagonists and partial agonists; bile acid transporter inhibitors; and the like.

Where a second active compound is used in addition to an active compound, of formula (I) as described herein, the two compounds may be administered together in a single composition, concomitantly, or on separate dosing schedules.

Effect of Treatment with Compound of Formula (I) on Fasted Glucose, Non Fasted Glucose and Insulin Tolerance Test in Diet Induced Obese Mice

### Objective

To assess the effect of Compound of formula (I) (200 mg/kg, i.p.) on fasted and non-fasted blood glucose and insulin tolerance test in high fat fed C57BL/6 male mice.

### Animals and Diet:

In house bred C57BL/6 male mice (about 8 weeks old) were put on high fat diet. Diets for these animals were purchased from Research Diet, USA.

### Materials:

Human Actrapid®, Accu-Check Sensor® glucometer and glucometer strips.

### Method:

Blood was collected by tail snip for the estimation of non-fasted and 4 hr fasted blood glucose (day 0) using an Accu-Check Sensor glucometer. Animals were then randomized into two groups, Compound of formula (I) (Compound No. 28) treated (200 mg/kg, i.p. for 4 days) and vehicle control group (10 ml/kg, i.p. for 4 days) with their non-fasted and 4 hr fasted blood glucose evenly matched.

On day 2 post dosing, 4 hr fasted blood glucose level was estimated.

Similarly on day 4, one hour post dosing, non fasted blood glucose value was estimated and then the animals were subjected to insulin tolerance test. Blood samples were collected for glucose estimation at 15, 30, 60 and 120 min. post insulin challenge.

### Analysis

Percent change in fasted and non-fasted blood glucose was calculated from their corresponding baseline blood glucose values (day 0). In the insulin tolerance test, the glucose values at various time points during were plotted against time and the AUC blood glucose was calculated.

### Results:

### Fasted and Non-Fasted Blood Glucose

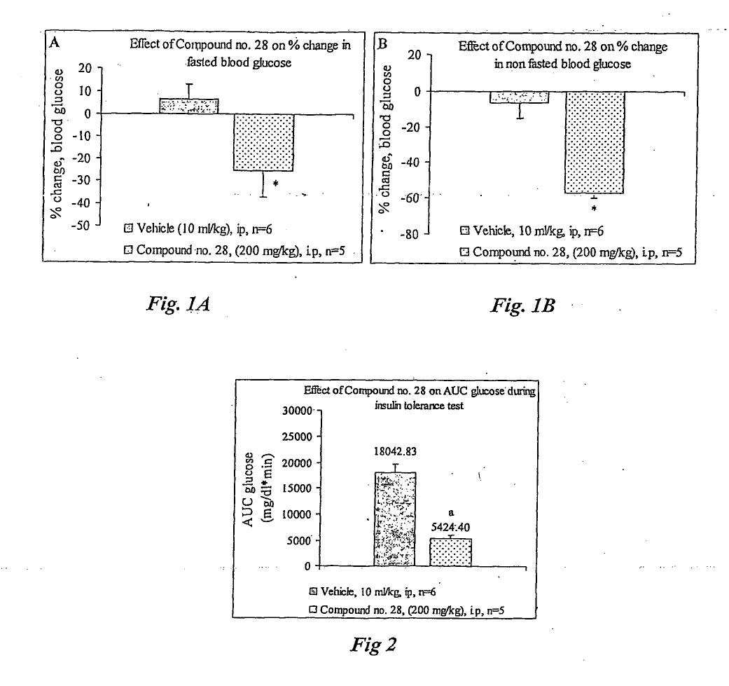

The percent change in fasted and non-fasted blood glucose values in the treatment group were significantly decreased as compared to the vehicle control group (FIG. 1, A and B)

### Intraperitoneal Insulin Tolerance Test (ITT)

During the ITT performed at the end of 4 days of treatment, Compound treated animals showed significant decreases in blood glucose post insulin challenge as compared to vehicle treated animals indicating improved insulin sensitivity as seen from AUC blood glucose (FIG. 2)

### Discussion:

Various researchers have reported that Protein Tyrosine Phosphatases particularly PTP-1B function as negative regulators of insulin signaling cascade, which leads to suppression of insulin action. Reducing PTP1B abundance not only enhances insulin sensitivity and improves glucose metabolism but also protects against obesity induced by high fat diet (Barry J. Goldstein, 2002). High fat fed C57BL/6 mice are prone to develop obesity, hyperinsulinemia, insulin resistance glucose intolerance, and diabetes (Bo Ahren et al 2004). Compounds of formula (I) inhibit PTP-1B enzyme in the in vitro assay. During in vivo studies, compound of formula (I) (compound no 28) has shown significant improvement in fasted and non-fasted blood glucose and has improved insulin sensitivity as seen from insulin tolerance test. Thus it can be concluded that compound of formula (I) by virtue of its inhibitory action on enzyme Protein Tyrosine Phosphatase (PTP-1B) are promising for the treatment and prevention of diseases mediated by PTP 1B, particularly diabetes, insulin resistance.

### REFERENCES

Barry J. Goldstein (2002) The Journal of Endocrinology & Metabolism 87(6), 2474-2480
Bo Ahrein. et al. (2004) Diabetes. 53 (3): S215-S219
Cheng et al. (2002) Developmental Cell 2, 497-503
Elchebly et al. (1999) Science 283, 1544-1548
Flint A J et. Al. (1993) The EMBO J. 12, 1937-1946
Gum R J. et al. (2003) Diabetes 52, 21-28
Hubbard S R. (1997) The EMBO J. 16 (18), 5573-81
Kishor M. Wasan & Norbert A. Looije (2005) J Plharn Pharmaceut Sci (www.cspscanada.org) 8(2), 259-271
Klaman et al. (2000) Molecular & Cellular Biology 20(15), 5479-5489
Mauro L J et al. (1994) J Biol Chem 269, 30659-30667
Moller et al. (2000) Current Opinion in Drug Discovery & Development 3 (5), 527-540
Noguchi T. et al. (1994) Mol. Cell. Biol. 14, 6674-6682
Rocchini A P. (2002) NEJM 346, 854-55
Rondinone et al. (2002) Diabetes 51, 2405-2411
Sinha R. et al. (2002) NEJM 346, 802-10
Taylor S D. & Hill B. (2004) Expert Opin. Investig. Drugs 13(3), 199-214
Wiener J R. et al. (1994) J. Natl. Cancer Inst. 86 (5): 372-378
Zinker et al. (2002) PNAS 99 (17), 11357-11362
Biochemical Pharmacology 54: 703-711 (1997).

From the foregoing description, one skilled in the art can easily ascertain the essential characteristics of this invention, and without departing from the spirit and scope thereof, can make various changes and modifications of the invention to adapt it to various usages and conditions.

1. A compound represented by formula (I), 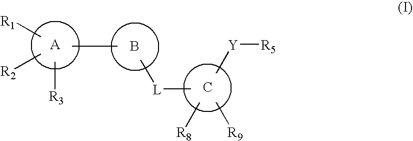
Wherein;
‘A’ is a member selected from the group consisting of: 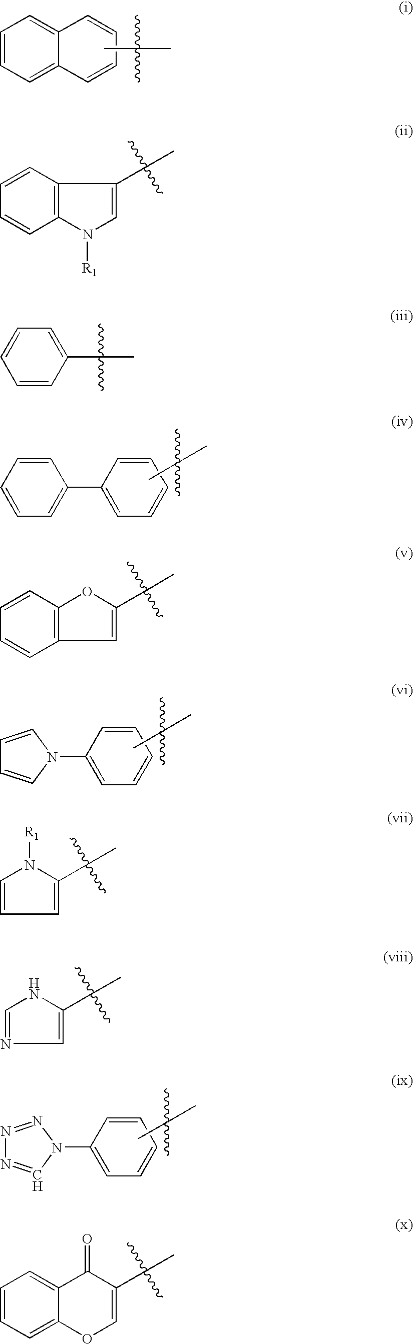 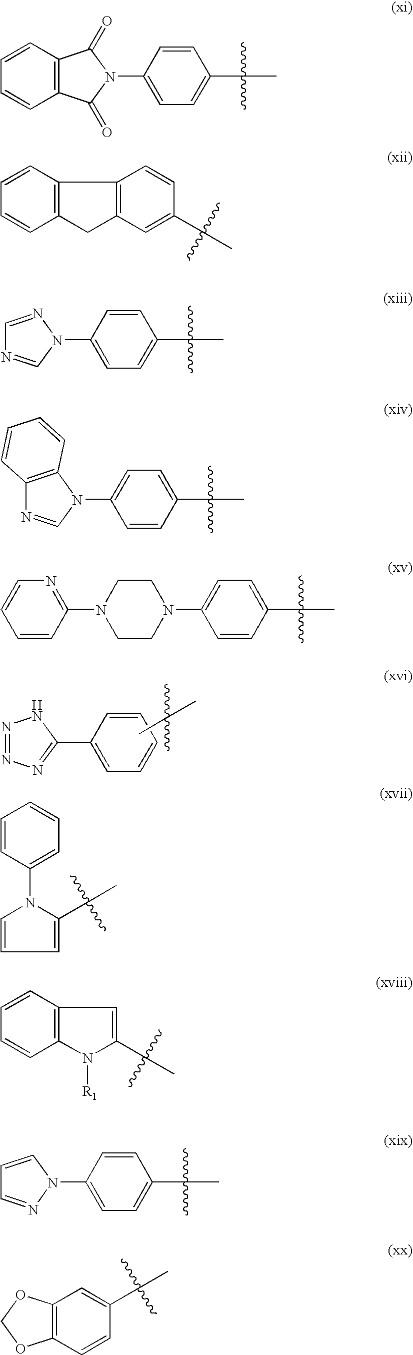 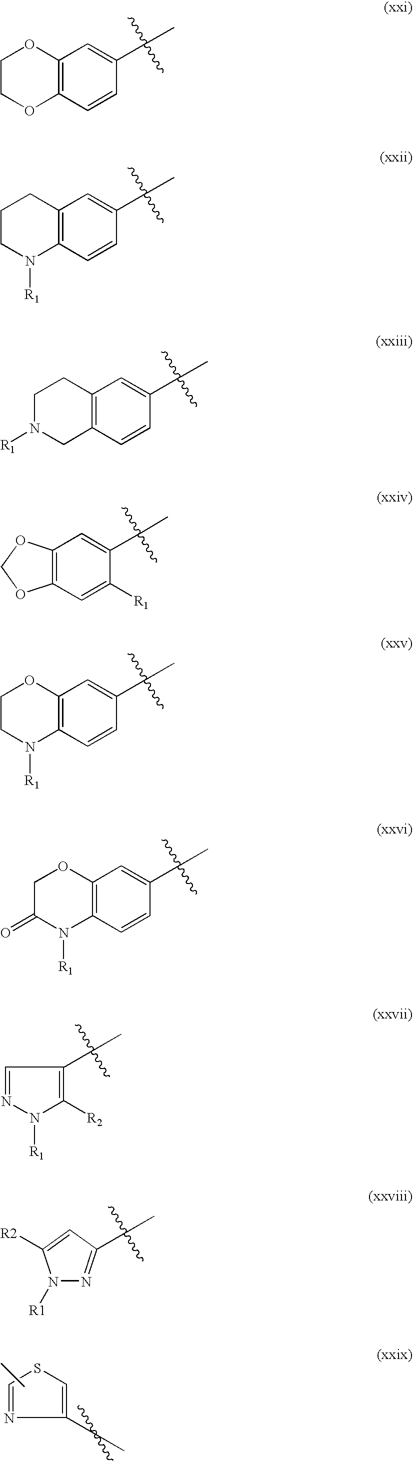 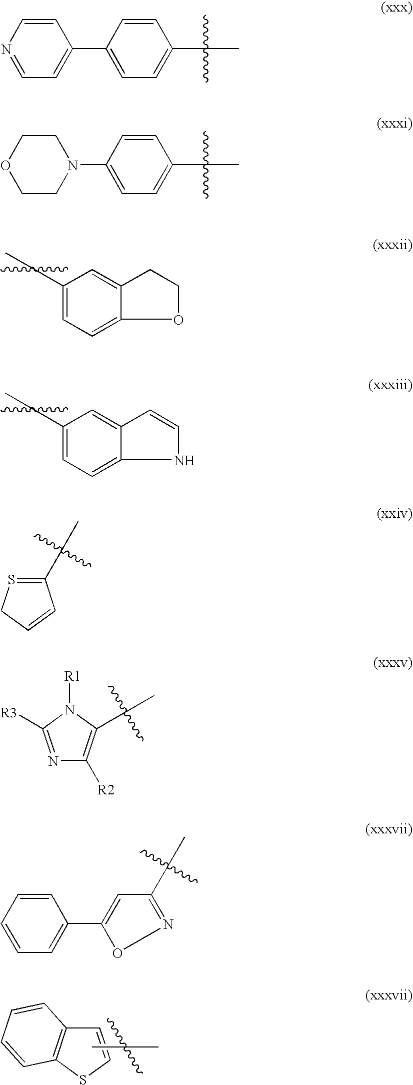
‘B’ is a group selected from: 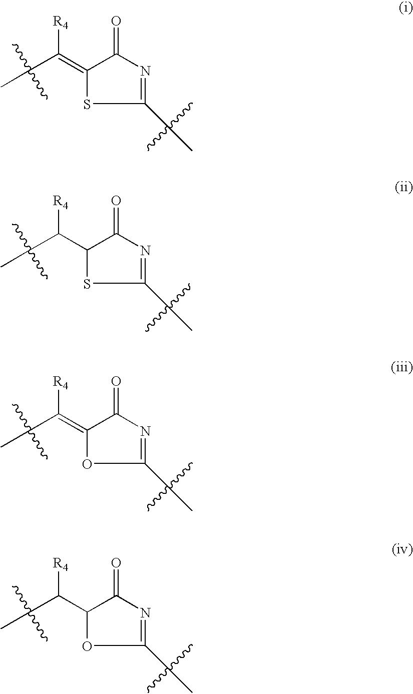
‘L,’ is selected from —NH—, —NH—CH₂—, —NH—CH(CH₃)—, —NH—CH—C(O)NH—, —NH—CH₂—CH₂—, —NHNH—, —NH—CH(COOH)—CH₂, —N(CH₂COOH)—;
‘C’ is a member selected from the group consisting of:
i) Phenyl ii) Naphthyl iii) Indolyl iv) Thiazolyl v) Benzimidazolyl
‘Y’ is selected from the group consisting of: 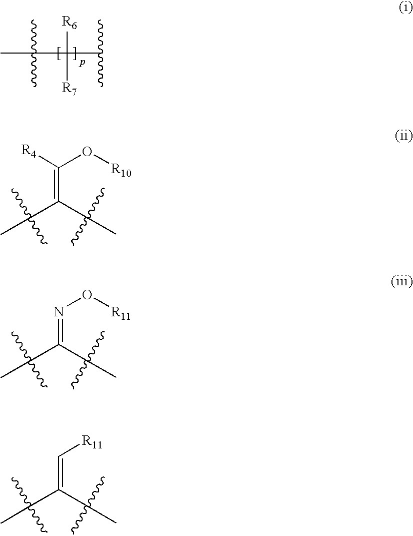
p is 1, 2 or 3;
R₁ is selected from the group consisting of:
i) Hydrogen ii) —CH₂COOR₄ iii) —X(CH₂)ₙ-aryl, where n is selected from 0, 1 or 2, and X is selected from S, NH or O, iv) —S-allyl, v) —OR₄ vi) OCH2cycloalkyl, wherein cycloalkyl is selected from cyclopropyl, cyclobutyl, cyclopentyl, cyclohexyl, cycloheptyl or cyclooctyl vii) —CH₂CONH₂, viii) —CN, ix) Halo x) alkyl xi) —SO₂aryl xii) —CF₃ xiii) —CO₂R₄ xiv) —COalkyl xv) —NH₂ xvi) —NO₂ xvii) —CH₂CONH-PhCH₂COOR₄ xviii) Tetrazolyl xix) Triazolyl xx) Pyrrolyl xxi) Benzimidazolyl xxii) Pyrazolyl xxiii) Phthalamoyl xxiv) Phenyl optionally substituted with one or more group selected from cyano, amino, carboxyl halo, nitro, alkyl, hydroxyl, —O-alkyl xxv) Biphenyl xxvi) —OCH₂CO₂R₄ xxvii) Pyridyl xxviii) Thienyl xxix) Dimethylamino xxx), —CH₂OH xxxi) —CF₂H xxxii) —SO₂N xxxiii) NHCOalkyl xxxiv) —OCF₃ 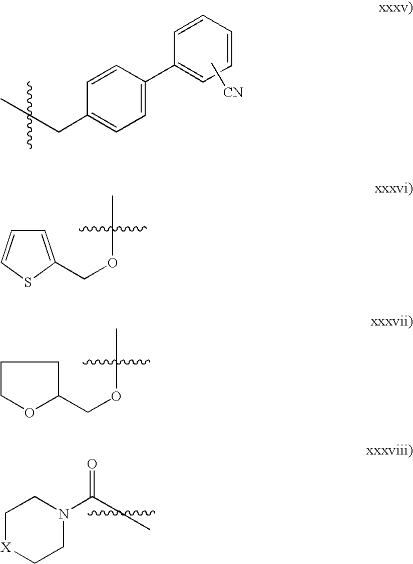
wherein x is as defined above;
R₂ and R₃ are independently selected from the group consisting of:
i) Hydrogen ii) —CH₂COOR₄ iii) —S-alkyl iv) —OR₄ v) —CH₂CONH₂ yl) —CN, vii) Halo viii) allyl ix) —CF₃ x) —COOR₄ xi) aryl xii) —COalkyl xiii) —NH₂ xiv) —NO₂ xv) —CF₂H xvi) thienyl xvii) NHCOalkyl xviii) —OCF₃;
R₄ is selected from hydrogen or allyl;
R₅ is selected from the group consisting of
i) —COOR₄ wherein R₄ is as defined above
ii) —C(O)C(O)OH, 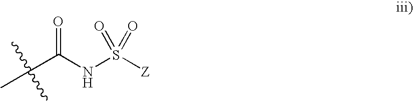
wherein z is selected from aryl or heteroaryl, optionally substituted by a group independently selected from H, alkyl, Halo, nitro, —OR₄, CF₃ and CONHR₄, 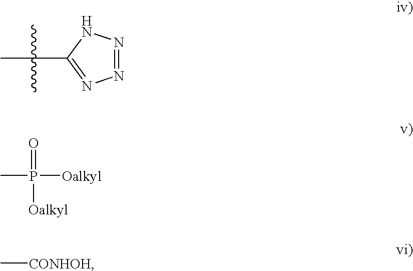
vii) —SO₂NHCOalkyl, viii) —SO₂NHalkyl, ix) —CONHSO₂alkyl, x) OH, xi) —NHCOalkyl, xii) —SO₂NHR₄;
R₆ is selected from the group consisting of
i) hydrogen,
ii) —OR₄,
iii) alkyl,
iv) halo,
v) heterocyclyl,
vi) —S alkyl,
vii) —S-aryl,
viii) —COOH,
ix) Benzyl,
x) heteroaryl;
R₇ is selected from the group consisting of
i) allyl,
ii) hydrogen,
iii) halo;
or R₆ and R₇ together may form cycloalkane ring selected from the group of cyclopentane, cyclohexane, cyclobutane;
R₈ and R₉ are independently selected from the group consisting of
i) hydrogen,
ii) halo,
iii) —OR₄,
iv) alkyl,
v) —CONHR₄,
i) —COalkyl;
R₁₀ is selected from the group consisting of
i) Alkyl, ii) Aryl;
R₁₁ is selected from the group consisting of
i) hydrogen,
ii) alkyl,
iii) aryl;
and with the proviso that:
i) when R₅ is —C(O)C(O)OH, then ‘Y’ is (i), and p is null
ii) when A is phenyl and is substituted by R₁ at fourth position then R₁ is not —X(CH₂)ₙ-aryl;
or a pharmaceutically acceptable salt or prodrug thereof.

2. A compound of the formula (I) as claimed in claim 1, wherein:
A is selected from the group consisting of: 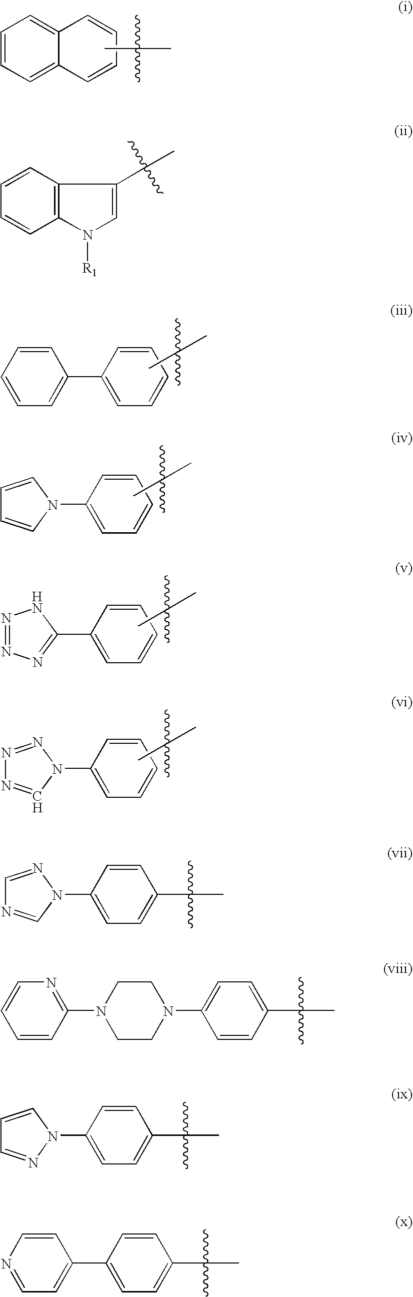
‘B’ is a group selected from 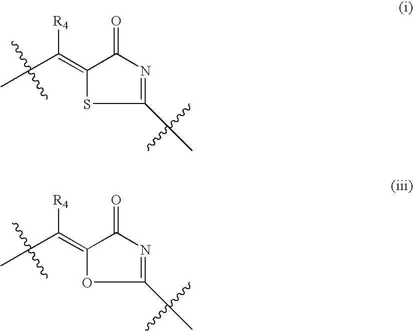
‘C’ is a member selected from the group consisting of:

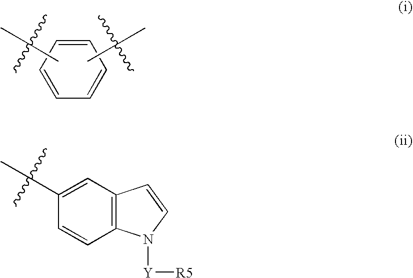

3. A compound of the formula (I) as claimed in claim 1, wherein:
‘A’ is a member selected from the group consisting of: 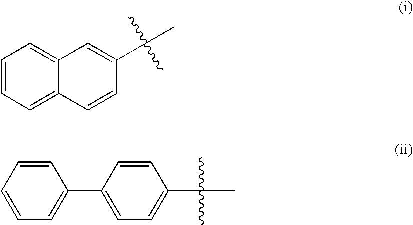
‘B’ is a group selected from 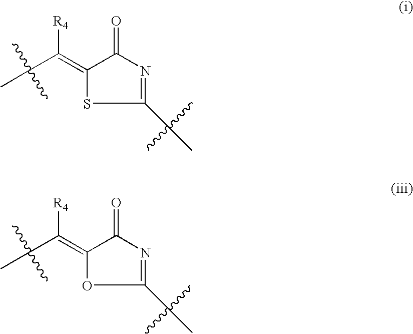
‘L’ is —NH—;
‘C’ is 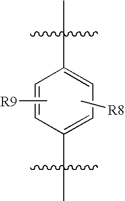
‘Y’ is 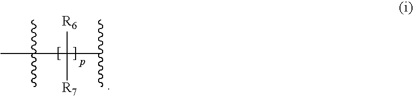
p is 1;
R₅ is selected from the group consisting of 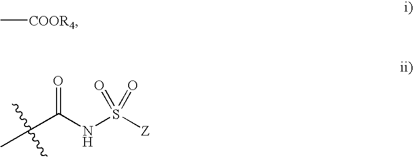
wherein z is selected from aryl or heteroaryl,
independently selected from H, alkyl, Halo, nitro, —OR₄, CF₃ and CONHR₄;
R₆ and R₇ are independently selected from hydrogen or halogen;
R₈ and R₉ are independently selected from the group consisting of
i) hydrogen,
ii) halo,
iii) —OR₄,
iv) alkyl,
v) —CONHR₄
vi) COOR₄,
vii) —COalkyl;

4. The compound as claimed in claim 1, wherein said compound is selected from the group consisting of:
[4-(5-Naphthalen-2-ylmethylene-4-oxo-4,5-dihydro-thiazol-2-ylamino)-phenyl]-acetic acid. (Compound no. 1)
{4-[5-(1-Carboxymethyl-1H-indol-3-ylmethylene)-4-oxo-4,5-dihydro-thiazol-2-ylamino]-phenyl}-acetic acid. (Compound no. 2)
{4-[5-(3-Benzyloxy-naphthalen-2-ylmethylene)-4-oxo-4,5-dihydro-thiazol-2-ylamino]-phenyl}-acetic acid. (Compound no. 3)
{4-[5-(4-Methylsulfanyl-benzylidene)-4-oxo-4,5-dihydro-thiazol-2-ylamino]-phenyl}-acetic acid. (Compound no. 4)
[4-(5-Benzylidene-4-oxo-4,5-dihydro-thiazol-2-ylamino)-phenyl]-acetic acid. (Compound no. 5)
{4-[S-(4-Methoxy-naphthalen-1-ylmethylene)-4-oxo-4,5-dihydro-thiazol-2-ylamino]-phenyl}-acetic acid. (Compound no. 6)
{4-[5-(6-Methoxy-naphthalen-2-ylmethylene)-4-oxo-4,5-dihydro-thiazol-2-ylamino]-phenyl}-acetic acid. (Compound no. 7)
{4-[5-(1H-Indol-3-ylmethylene)-4-oxo-4,5-dihydro-thiazol-2-ylamino]-phenyl}-acetic acid. (Compound no. 8)
[4-(5-Biphenyl-4-ylmethylene-4-oxo-4,5-dihydro-thiazol-2-ylamino)-phenyl]-acetic acid. (Compound no. 9)
{4-[5-(3-Isopropoxy-naphthalen-2-ylmethylene)-4-oxo-4,5-dihydro-thiazol-2-ylamino]-phenyl}-acetic acid. (Compound no. 10)
{4-[5-(1-tert-Butoxycarbonylmethyl-1H-indol-3-ylmethylene)-4-oxo-4,5-dihydro-thiazol-2-ylamino]-phenyl}-acetic acid. (Compound no. 11)
[4-(4-Oxo-5-quinolin-2-ylmethylene-4,5-dihydro-thiazol-2-ylamino)-phenyl]-acetic acid. (Compound no. 12)
{4-[5-(1-Benzofuran-2-yl-ethylidene)-4-oxo-4,5-dihydro-thiazol-2-ylamino]-phenyl}-acetic acid. (Compound no. 13)
{3-[5-(3-Benzyloxy-naphthalen-2-ylmethylene)-4-oxo-4,5-dihydro-thiazol-2-ylamino]-phenyl}-acetic acid. (Compound no. 14)
{4-[5-(1-Carbamoylmethyl-1H-indol-3-ylmethylene)-4-oxo-4,5-dihydro-thiazol-2-ylamino]-phenyl}-acetic acid. (Compound no. 15)
[3-(5-Naphthalen-2-ylmethylene-4-oxo-4,5-dihydro-thiazol-2-ylamino)-phenyl]-acetic acid. (Compound no. 16)
{4-[5-(2′-Cyano-biphenyl-4-ylmethylene)-4-oxo-4,5-dihydro-thiazol-2-ylamino]-phenyl}-acetic acid. (Compound no. 17)
{4-[4-Oxo-5-(4-pyrrol-1-yl-benzylidene)-4,5-dihydro-thiazol-2-ylamino]-phenyl}-acetic acid. (Compound no. 18)
(4-{4-Oxo-5-[4-(1H-tetrazol-5-yl)-benzylidene]-4,5-dihydro-thiazol-2-ylamino}-phenyl)-acetic acid. (Compound no. 19)
(4-{5-[2-But yl-5-chloro-3-(2′-cyano-biphenyl-4-ylmethyl)-3H-indazol-4-ylmethylene]-4-oxo-4,5-dihydro-thiazol-2-ylamino}-phenyl)-acetic acid. (Compound no. 20)
{3-[5-(3-Isopropoxy-naphthalen-2-ylmethylene)-4-oxo-4,5-dihydro-thiazol-2-ylamino]-phenyl}-acetic acid. (Compound no. 21)
(4-{2-[5-(3-Benzyloxy-naphthalen-2-ylmethylene)-4-oxo-4,5-dihydro-thiazol-2-ylamino]-acetyl amino}-phenyl)-acetic acid. (Compound no. 22)
{3-[5-(1-Carbamoylmethyl-1H-indol-3-ylmethylene)-4-oxo-4,5-dihydro-thiazol-2-ylamino]-phenyl}-acetic acid. (Compound no. 23)
[4-(S-Naphthalen-2-ylmethylene-4-oxo-4,5-dihydro-thiazol-2-ylamino)-benzyl]-phosphonic acid diethyl ester. (Compound no. 24)
4-Methyl-N-{2-[4-(5-naphthalen-2-ylmethylene-4-oxo-4,5-dihydro-thiazol-2-ylamino)-phenyl]-acetyl}-benzene sulfonamide. (Compound no. 25)
N-(2-{4-[5-(3-Benzyloxy-naphthalen-2-ylmethylene)-4-oxo-4,5-dihydro-thiazol-2-ylamino]-phenyl}-acetyl)-4-methyl-benzenesulfonamide. (Compound no. 26)
N-{2-[4-(5-Biphenyl-4-ylmethylene-4-oxo-4,5-dihydro-thiazol-2-ylamino)-phenyl]-acetyl}-4-methyl-benzenesulfonamide. (Compound no. 27)
N-{2-[4-(5-Biphenyl-4-ylmethlene-4-oxo-4,5-dihydro-thiazol-2-ylamino)-phenyl]-acetyl}-4-methyl-benzenesulfonamide sodium salt. (Compound no. 28)
(4-{4-Oxo-5-[1-(toluene-4-sulfonyl)-1H-indol-3-ylmethylene]-4,5-dihydro-thiazol-2-ylamino}-phenyl)-acetic acid. (Compound no. 29)
(3-{4-Oxo-5-[1-(toluene-4-sulfonyl)-1H-indol-3-ylmethylene]-4,5-dihydro-thiazol-2-ylamino}-phenyl)-acetic acid. (Compound no. 30)
{4-[5-(3,4-Dichloro-benzylidene)-4-oxo-4,5-dihydro-thiazol-2-ylamino]-phenyl}-acetic acid. (Compound no. 31)
{4-[5-(3-Hydroxy-naphthalen-2-ylmethylene)-4-oxo-4,5-dihydro-thiazol-2-ylamino]-phenyl}-acetic acid. (Compound no. 32)
5-(3-Benzyloxy-naphthalen-2-ylmethylene)-2-[4-(1H-tetrazol-5-ylmethyl)-phenylamino]-thiazol-4-one. (Compound no. 33)
[4-(N′-{4-Oxo-5-[4-(1H-tetrazol-5-yl)-benzylidene]-4,5-dihydro-thiazol-2-yl}-hydrazino)-phenyl]-acetic acid. (Compound no. 34)
2-[4-(5-Biphenyl-4-ylmethylene-4-oxo-4,5-dihydro-thiazol-2-ylamino)-phenyl]-N-hydroxy-acetamide. (Compound no. 35)
(4-{5-[4-(Morpholine-4-carbonyl)-benzylidene]-4-oxo-4,5-dihydro-thiazol-2-ylamino}-phenyl)-acetic acid. (Compound no. 36)
{4-[5-(3,5-Di-tert-butyl-4-hydroxy-benzylidene)-4-oxo-4,5-dihydro-thiazol-2-ylamino]-phenyl}-acetic acid. (Compound no. 37)
{4-[5-(2-Butyl-5-chloro-3H-imidazol-4-ylmethylene)-4-oxo-4,5-dihydro-thiazol-2-ylamino]-phenyl}-acetic acid. (Compound no. 38)
(4-{5-[4-(2-Fluoro-4-nitro-phenoxy)-benzylidene]-4-oxo-4,5-dihydro-thiazol-2-ylamino}-phenyl)-acetic acid. (Compound no. 39)
(4-{4-Oxo-5-[4-(piperazine-1-carbonyl)-benzylidene]-4,5-dihydro-thiazol-2-ylamino}-phenyl)-acetic acid. (Compound no. 40)
5-(6-Methoxy-naphthalen-2-ylmethylene)-2-[4-(1H-tetrazol-5-ylmethyl)-phenylamino]-thiazol-4-one. (Compound no. 41)
3-[4-(a-Naphthalen-2-ylmethylene-4-oxo-4,5-dihydro-thiazol-2-ylamino)-phenyl]-prop ionic acid. (Compound no. 42)
3-{4-[5-(1-Carboxymethyl-1H-indo-3-ylmethylene)-4-oxo-4,5-dihydro-thiazol-2-ylamino]-phenyl}-p)propionic acid. (Compound no. 43)
{3-[2-(4-Methylsulfamoylmethyl-phenylamino)-4-oxo-4H-thiazol-5-ylidenemethyl]-indol-1-yl}-acetic acid. (Compound no. 44)
{4-[4-Oxo-5-(4-tetrazol-1-yl-benzylidene)-4,5-dihydro-thiazol-2-ylamino]-phenyl}-acetic acid. (Compound no. 45)
3-{4-[5-(6-Methoxy-naphthalen-2-ylmethylene)-4-oxo-4,5-dihydro-thiazol-2-ylamino]-phenyl}-propionic acid. (Compound no. 46)
{5-[5-(3-Benzyloxy-naphthalen-2-ylmethylene)-4-oxo-4,5-dihydro-thiazol-2-ylamino]-indol-1-yl}-acetic acid. (Compound no. 47)
2-{4-[5-(3-Benzyloxy-naphthalen-2-ylmethylene)-4-oxo-4,5-dihydro-thiazol-2-ylamino]-phenyl}-butyric acid. (Compound no. 48)
3-{4-[5-(3-Benzyloxy-naphthalen-2-ylmethylene)-4-oxo-4,5-dihydro-thiazol-2-ylamino]-phenyl}-propionic acid. (Compound no. 49)
(4-{5-[3-(1,3-Dioxo-1,3-dihydro-isoindol-2-yl)-benzylidene]-4-oxo-4,5-dihydro-thiazol-2-ylamino}-phenyl)-acetic acid. (Compound no. 50)
{4-[4-Oxo-5-(3-pyrrol-1-yl-benzylidene)-4,5-dihydro-thiazol-2-ylamino]-phenyl}-acetic acid. (Compound no. 51)
N-Methyl-C-[4-(5-naphthalen-2-ylmethylene-4-oxo-4,5-dihydro-thiazol-2-ylamino)-phenyl]-methanesulfonamide. (Compound no. 52)
2-[4-(5-Naphthalen-2-yl-ethylene-4-oxo-4,5-dihydro-thiazol-2-ylamino)-phenyl]-propionic acid. (Compound no. 53)
2-[4-(5-Biphenyl-4-ylmethylene-4-oxo-4,5-dihydro-thiazol-2-ylamino)-phenyl]-propionic acid. (Compound no. 54)
2-[4-(5-Biphenyl-4-ylmethylene-4-oxo-4,5-dihydro-thiazol-2-ylamino)-phenyl]-succinic acid. (Compound no. 55)
{4-[5-(4-hydroxy-3,5-dimethoxy-benzylidene)-4-oxo-4,5-dihydro-thiazol-2-ylamino]-phenyl}-acetic acid. (Compound no. 56)
(4-{5-[1-(6-Methoxy-naphthalen-2-yl)-ethylidene]-4-oxo-4,5-dihydro-thiazol-2-ylamino}-phenyl)-acetic acid. (Compound no. 57)
3-{4-[5-(3,5-Di-tert-butyl)-hydroxy-benzylidene)-4-oxo-4,5-dihydro-thiazol-2-ylamino]-phenyl}-prop ionic acid. (Compound no. 58)
{4-[4-Oxo-5-(3,4,5-trimethoxy-benzylidene)-4,5-dihydro-thiazol-2-ylamino]-phenyl}-acetic acid. (Compound no. 59)
(4-{5-[1-(4-Bromo-phenyl)-1H-pyrrol-2-ylmethylene]-4-oxo-4,5-dihydro-thiazol-2-ylamino}-phenyl)-acetic acid. (Compound no. 60)
(4-{4-Oxo-5-[1-(3-pyrrol-1-yl-phenyl)-ethylidene]-4,5-dihydro-thiazol-2-ylamino}-phenyl)-acetic acid. (Compound no. 61)
{4-[4-Oxo-5-(1-phenyl-1H-pyrrol-2-ylmethylene)-4,5-dihydro-thiazol-2-ylamino]-phenyl}-acetic acid. (Compound no. 62)
C-{4-[5-(6-Methoxy-naphthalen-2-ylmethylene)-4-oxo-4,5-dihydro-thiazol-2-ylamino]-phenyl}-N-methyl-methanesulfonamide. (Compound no. 63)
C-[4-(5-Biphenyl-4-ylmethylene-4-oxo-4,5-dihydro-thiazol-2-ylamino)-phenyl]-N-ethyl-methanesulfonamide. (Compound no. 64)
C-{4-[5-(2′-Cyano-biphenyl-4-ylmethylene)-4-oxo-4,5-dihydro-thiazol-2-ylamino]-phenyl}-N-methyl-methanesulfonamide. (Compound no. 65)
[4-(5-Biphenyl-4-ylmethylene-4-oxo-4,5-dihydro-thiazol-2-ylamino)-phenyl]-bromo-acetic acid. (Compound no. 66)
2-[4-(5-Biphenyl-4-ylmethylene-4-oxo-4,5-dihydro-thiazol-2-ylamino)-benzyl]-malonic acid. (Compound no. 67)
[4-(5-Biphenyl-4-ylmethylene-4-oxo-4,5-dihydro-thiazol-2-ylamino)-phenyl]-morpholin-4-yl-acetic acid. (Compound no. 68)
N-(2-{4-[5-(6-Methoxy-naphthalen-2-ylmethylene)-4-oxo-4,5-dihydro-thiazol-2-ylamino]-phenyl}-acetyl)-4-methyl-benzenesulfonamide sodium salt. (Compound no. 69)
5-Naphthalen-2-ylmethylene-2-[4-(1H-tetrazol-5-ylmethyl)-phenylamino]-thiazol-4-one. (Compound no. 70)
{4-[4-oxo-5-(4-oxo-4H-chromen-3-ylmethylene)-4,5-dihydro-thiazol-2-ylamino]-phenyl}-acetic acid. (Compound no. 71)
{4-[4-Oxo-5-(4-trifluoro methyl-benzylidene)-4,5-dihydro-thiazol-2-ylamino]-phenyl}-acetic acid. (Compound no. 72)
{4-[5-(6-Isopropoxy-naphthalen-2-ylmethylene)-4-oxo-4,5-dihydro-thiazol-2-ylamino]-phenyl}-acetic acid. (Compound no. 73)
[4-(5-Biphenyl-4-ylmethylene-4-oxo-4,5-dihydro-thiazol-2-ylamino)-phenyl]-acetic acid. (Compound no. 74)
[4-(5-Biphenyl-4-ylmethylene-4-oxo-4,5-dihydro-thiazol-2-ylamino)-phenyl]-bromo-sodium acetate. (Compound no. 75)
[4-(5-Biphenyl-4-ylmethylene-4-oxo-4,5-dihydro-thiazol-2-ylamino)-phenyl]-orpholin-4-yl-sodium acetate. (Compound no. 76)
2-[4-(5-Biphenyl-4-ylmethylene-4-oxo-4,5-dihydro-thiazol-2-ylamino)-phenyl]-propionic acid. (Compound no. 77)
[4-(5-Biphenyl-4-ylmethylene-4-oxo-4,5-dihydro-thiazol-2-ylamino)-phenyl]-sodium acetate. (Compound no. 78)
{4-[5-(3-Benzyloxy-naphthalen-2-ylmethylene)-4-oxo-4,5-dihydro-thiazol-2-ylamino]-phenyl}-sodium acetate. (Compound no. 79)
[4-(5-Naphthalen-2-ylmethylene-4-oxo-4,5-dihydro-thiazol-2-ylamino)-phenyl]-sodium acetate. (Compound no. 80)
{4-[5-(6-Methoxy-naphthalen-2-ylmethylene)-4-oxo-4,5-dihydro-thiazol-2-ylamino]-phenyl}-sodium acetate. (Compound no. 81)
{5-[5-(3-Benzyloxy-naphthalen-2-ylmethylene)-4-oxo-4,5-dihydro-thiazol-2-ylamino]-indol-1-yl}-sodium acetate. (Compound no. 82)
{4-[4-Oxo-5-(3-pyrrol-1-yl-benzylidene)-4,5-dihydro-thiazol-2-ylamino]-phenyl}-sodium acetate. (Compound no. 83)
2-{4-[L-(3-Benzyloxy-naphthalen-2-ylmethylene)-4-oxo-4,5-dihydro-thiazol-2-ylamino]-phenyl}-sodium butyrate. (Compound no. 84)
(4-{5-[3-(4-Fluoro-benzyloxy)-naphthalen-2-ylmethylene]-4-oxo-4,5-dihydro-thiazol-2-ylamino}-phenyl)-acetic acid. (Compound no. 85)
{4-[5-(9,9a-Dihydro-4aH-fluoren-2-ylmethylene)-4-oxo-4,5-dihydro-thiazol-2-ylamino]-phenyl}-acetic acid. (Compound no. 86)
{4-[4-Oxo-5-(3-[1,2,4]triazol-1-yl-benzylidene)-4,5-dihydro-thiazol-2-ylamino]-phenyl}-acetic acid. (Compound no. 87)
{4-[5-(3-Benzoimidazol-1-yl-benzylidene)-4-oxo-4,5-dihydro-thiazol-2-ylamino]-phenyl}-acetic acid. (Compound no. 88)
3-[4-(5-Naphthalen-2-yl methylene-4-oxo-4,5-dihydro-thiazol-2-ylamino)-phenyl]-sodium propionate. (Compound no. 89)
(4-{4-Oxo-5-[3-(4-pyridin-2-yl-piperazin-1-yl)-benzylidene]-4,5-dihydro-thiazol-2-ylamino}-phenyl)-acetic acid. (Compound no. 90)
{4-[4-Oxo-5-(3-pyrazol-1-yl-benzylidene)-4,5-dihydro-thiazol-2-ylamino]-phenyl}-acetic acid. (Compound no. 91)
{4-[4-Oxo-5-(3-tetrazol-1-yl-benzylidene)-4,5-dihydro-thiazol-2-ylamino]-phenyl}-sodium acetate. (Compound no. 92)
5-(3-Benzyloxy-naphthalen-2-ylmethylene)-2-[4-(5H-tetrazol-5-ylmethyl)-phenylamino]-thiazol-4-one sodium salt. (Compound no. 93)
[4-(5-Biphenyl-4-ylmethylene-4-oxo-4,5-dihydro-thiazol-2-ylamino)-phenyl]-acetic acid. (Compound no. 94)
[4-(5-Biphenyl-4-ylmethylene-4-oxo-4,5-dihydro-thiazol-2-ylamino)-phenyl]-sodium acetate. (Compound no. 95)
3-{4-[5-(3-Benzyloxy-naphthalen-2-ylmethylene)-4-oxo-4,5-dihydro-thiazol-2-ylamino]-phenyl}-sodium propionate. (Compound no. 96)
[5-(5-Biphenyl-4-ylmethylene-4-oxo-4,5-dihydro-thiazol-2-ylamino)-indol-1-yl]-sodium acetate. (Compound no. 97)
5-(6-Methoxy-naphthalen-2-ylmethylene)-2-[4-(5H-tetrazol-5-ylmethyl)-phenylamino]-thiazol-4-one sodium salt. (Compound no. 98)
{5-[5-(6-Methoxy-naphthalen-2-ylmethylene)-4-oxo-4,5-dihydro-thiazol-2-ylamino]-indol-1-yl}-sodium acetate. (Compound no. 99)
[4-(5-Biphenyl-4-ylmethylene-4-oxo-4,5-dihydro-oxazol-2-ylamino)-phenyl]-acetic acid. (Compound no. 100)
[4-(5-Biphenyl-4-ylmethylene-4-oxo-4,5-dihydro-oxazol-2-ylamino)-phenyl]-sodium acetate. (Compound no. 101)
N-(2-{4-[4-Oxo-5-(4-phenyl-cyclohexa-1,5-dienylmethylene)-4,5-dihydro-thiazol-2-ylamino]-phenyl}-acetyl)-benzenesulfonamide sodium salt. (Compound no. 102)
N-(2-{4-[4-Oxo-5-(4-pyrazol-1-yl-benzylidene)-4,5-dihydro-thiazol-2-ylamino]-phenyl}-acetyl)-benzenesulfonamide. (Compound no. 103)
N-(2-{4-[5-(3-Benzyloxy-naphthalen-2-ylmethylene)-4-oxo-4,5-dihydro-thiazol-2-ylamino]-phenyl}-acetyl)-4-methyl-benzenesulfonamide sodium salt. (Compound no. 104)
N-{2-[4-(5-Biphenyl-4-ylmethylene-4-oxo-4,5-dihydro-thiazol-2-ylamino)-phenyl]-acetyl}-4-chloro-benzenesulfonamide sodium salts. (Compound no. 105)
4-Chloro-N-(2-{4-[5-(6-methoxy-naphthalen-2-ylmethylene)-4-oxo-4,5-dihydro-thiazol-2-ylamino]-phenyl}-acetyl)-benzenesulfonamide sodium salt. (Compound no. 106)
4-Chloro-N-(2-{4-[4-oxo-5-(4-pyrazol-1-yl-benzylidene)-4,5-dihydro-thiazol-2-ylamino]-phenyl}-acetyl)-benzenesulfonamide sodium salt. (Compound no. 107)
N-(2-{4-[5-(3,5-Di-tert-butyl-4-hydroxy-benzylidene)-4-oxo-4,5-dihydro-thiazol-2-ylamino]-phenyl}-acetyl)-4-methyl-benzenesulfonamide. (Compound no. 108)
[4-(4-Oxo-5-1,1′;4′,1″]terphenyl-ylmethylene-4,5-dihydro-thiazol-2-ylamino)-phenyl]-acetic acid. (Compound no. 109)
{4-[5-(6-Bromo-naphthalen-2-ylmethylene)-4-oxo-4,5-dihydro-thiazol-2-ylamino]-phenyl}-sodium acetate. (Compound no. 110)
[4-(5-Naphthalen-2-ylmethylene-4-oxo-4,5-dihydro-thiazol-2-ylamino)-phenyl]-oxo-acetic acid. (Compound No. 111)
4-Methyl-N-{2-[4-(5-naphthalen-2-ylmethylene-4-oxo-4,5-dihydro-thiazol-2-ylamino)-phenyl]-acetyl}-benzenesulfonamide sodium salt. (Compound no. 112)
(4-{5-[3-(4-Fluoro-benzyloxy)-naphthalen-2-ylmethylene-4-oxo-4,5-dihydro-thiazol-2-ylamino)-phenyl]-acetyl}-sodium acetate. (Compound no. 113)
C-{4-[5-(3-Benzyloxy-naphthalen-2-ylmethylene)-4-oxo-4,5-dihydro-thiazol-2-ylamino]-phenyl}-N-methyl-methanesulfonamide. (Compound no. 114)
{4-[5-(2-Benzyloxy-benzylidene)-4-oxo-4,5-dihydro-thiazol-2-ylamino]-phenyl}-sodium acetate. (Compound no. 115)
{4-[5-(3-Methoxy-naphthalen-2-ylmethylene)-4-oxo-4,5-dihydro-thiazol-2-ylamino]-phenyl}-sodium acetate. (Compound no. 116)
{-[5-(3-Ethoxy-naphthalen-2-ylmethylene)-4-oxo-4,5-dihydro-thiazol-2-ylamino]-phenyl}-sodium acetate. (Compound no. 117)
N-(2-{4-[5-(2-Benzyloxy-benzylidene)-4-oxo-4,5-dihydro-thiazol-2-ylamino]-phenyl}-acetyl)-4-methyl-benzenesulfonamide sodium salt. (Compound no. 118)
{4-[5-(2-Benzyloxy-4-pyrrol-1-yl-benzylidene)-4-oxo-4,5-dihydro-thiazol-2-ylamino]-phenyl}-sodium acetate. (Compound no. 119)
(4-{5-[2-(2′-Cyano-biphenyl-4-ylmethoxy)-benzylidene]-4-oxo-4,5-dihydro-thiazol-2-ylamino}-phenyl)-sodium acetate. (Compound no. 120)
[4-(5-Benzo[1,3]dioxol-5-ylmethylene-4-oxo-4,5-dihydro-thiazol-2-ylamino)-phenyl]-acetic acid. (Compound no. 121)
{4-[5-(2-Benzyloxy-4-tetrazol-1-yl-benzylidene)-4-oxo-4,5-dihydro-thiazol-2-ylamino]-phenyl}-sodium acetate. (Compound no. 122)
{4-[5-(2,3-Dihydro-benzo[1,4]dioxin-6-ylmethylene)-4-oxo-4,5-dihydro-triazol-2-ylamino]-phenyl}-acetic acid. (Compound no. 123)
{4-[4-Oxo-5-(1-phenyl-5-pyrrol-1-yl-1H-pyrazol-3-ylmethylene)-4,5-dihydro-thiazol-2-ylamino]-phenyl}-acetic acid. (Compound no. 124)
N-(2-{4-[5-(2,3-Dihydro-benzo[1,4]dioxin-6-ylmethylene)-4-oxo-4,5-dihydro-thiazol-2-ylamino]-phenyl}-acetyl)-4-methyl-benzenesulfonamide. (Compound no. 125)
N-(2-{4-[5-(2,3-Dihydro-benzo[1,4dioxin-6-ylmethylene]-4-oxo-4,5-dihydro-thiazol-2-ylamino]-phenyl}-acetyl)-4-methyl-benzenesulfonamide. (Compound no. 126)
N-(2-{4-[5-(2-Benzyloxy-4-tetrazol-1-yl-benzylidene)-4-oxo-4,5-dihydro-thiazol-2-ylamino]-phenyl}-acetyl)-4-methyl-benzenesulfonamide sodium salt. (Compound no. 127)
[2-(5-Naphthalen-2-ylmethylene-4-oxo-4,5-dihydro-thiazol-2-ylamino)-thiazol-4-yl]-sodium acetate. (Compound no. 128)
{4-[4-Oxo-5-(3-phenethyloxy-naphthalen-2-ylmethylene)-4,5-dihydro-thiazol-2-ylamino]-phenyl}-sodium acetate. (Compound no. 129)
[2-(5-Biphenyl-4-ylmethylene-4-oxo-4,5-dihydro-thiazol-2-ylamino)-thiazol-4-yl]-acetic acid. (Compound no. 130)
N-(2-{4-[5-(3-Benzyloxy-4-tetrazol-1-yl-benzylidene)-4-oxo-4,5-dihydro-thiazol-2-ylamino]-phenyl}-acetyl)-4-methyl-benzenesulfonamide sodium salt (Compound no. 131)
{4-[5-(5-Methyl-1-phenyl-1H-pyrazol-3-ylmethylene)-4-oxo-4,5-dihydro-thiazol-2-ylamino]-phenyl}-acetic acid. (Compound no. 132)
{2-[5-(3-Benzyloxy-naphthalen-2-ylmethylene)-4-oxo-4,5-dihydro-thiazol-2-ylamino]-thiazol-4-yl}-acetic acid. (Compound no. 133)
{2-[5-(5-Methyl-1-phenyl-1H-pyrazol-3-ylmethylene)-4-oxo-4,5-dihydro-thiazol-2-ylamino]-thiazol-4-yl}-acetic acid. (Compound no. 134)
{4-[5-(3-Cyclopentylmethoxy-naphthalen-2-ylmethylene)-4-oxo-4,5-dihydro-thiazol-2-ylamino]-phenyl}-sodium acetate. (Compound no. 135)
2-{4-[5-(3-Benzyloxy-naphthalen-2-ylmethylene)-4-oxo-4,5-dihydro-thiazol-2-ylamino]-phenyl}-3-phenyl-sodium propionate. (Compound no. 136)
{4-[5-(4-Fluoro-benzylidene)-4-oxo-4,5-dihydro-thiazol-2-ylamino]-phenyl}-acetic acid. (Compound no. 137)
{4-[4-Oxo-5-(3-propoxy-naphthalen-2-ylmethylene)-4,5-dihydro-thiazol-2-ylamino]-phenyl}-acetic acid. (Compound no. 138)
{4-[5-(1-Isopropyl-1,2,3,4-tetrahydro-quinolin-6-ylmethylene)-4-oxo-4,5-dihydro-thiazol-2-ylamino]-phenyl}-acetic acid. (Compound no. 139)
{4-[5-(3,5-Dipropoxy-naphthalen-2-ylmethylene)-4-oxo-4,5-dihydro-thiazol-2-ylamino]-phenyl}-acetic acid. (Compound no. 140)
{4-[5-(1-Methyl-1,2,3,4-tetrahydro-quinolin-6-ylmethylene)-4-oxo-4,5-dihydro-thiazol-2-ylamino]-phenyl}-acetic acid. (Compound no. 141)
(4-{4-Oxo-5-[3-(tetrahydro-furan-2-ylmethoxy)-naphthalen-2-ylmethylene]-4,5-dihydro-thiazol-2-ylamino}-phenyl)-acetic acid. (Compound no. 142)
4-[5-(6-Ethoxy-naphthalen-2-ylmethylene)-4-oxo-4,5-dihydro-thiazol-2-ylamino]-phenyl}-sodium acetate. (Compound no. 143)
{4-[5-(3,5-Diethoxy-naphthalen-2-ylmethylene)-4-oxo-4,5-dihydro-thiazol-2-ylamino]-phenyl}-sodium acetate. (Compound no. 144)
{4-[4-Oxo-5-(2-phenyl-thiazol-4-ylmethylene)-4,5-dihydro-thiazol-2-ylamino]-phenyl}-sodium acetate. (compound no. 145)
{4-[5-(1-Methoxy-naphthalen-2-ylmethylene)-4-oxo-4,5-dihydro-thiazol-2-ylamino]-phenyl}-acetic acid. (Compound no. 146)
{4-[4-Oxo-5-(4-tetrazol-1-yl-benzylidene)-4,5-dihydro-thiazol-2-ylamino]-phenyl}-sodium acetate. (Compound no. 147)
{4-[4-Oxo-5-(4-pyrrol-1-yl-benzylidene)-4,5-dihydro-thiazol-2-ylamino]-phenyl}-sodium acetate. (Compound no. 148)
{4-[5-(4-Benzyloxy-biphenyl-3-ylmethylene)-4-oxo-4,5-dihydro-thiazol-2-ylamino]-phenyl}-sodium acetate. (Compound no. 149)
{4-[5-(1-Ethoxy-naphthalen-2-ylmethylene)-4-oxo-4,5-dihydro-triazol-2-ylamino]-phenyl}-sodium acetate. (Compound no. 150)
{4-[5-(1-Benzyloxy-naphthalen-2-methylene)-4-oxo-4,5-dihydro-thiazol-2-ylamino]-phenyl}-sodium acetate. (Compound no. 151)
{4-[5-(4′-Methoxy-7-biphenyl-4-ylmethylene)-4-oxo-4,5-dihydro-thiazol-2-ylamino]-phenyl}-sodium acetate. (Compound no. 152)
[4-(5-[2,2′]Bithiophenyl-5-ylmethylene-4-oxo-4,5-dihydro-thiazol-2-ylamino)-phenyl]-sodium acetate. (Compound no. 153)
3-{4-[5-(1-Hydroxy-naphthalen-2-ylmethylene)-4-oxo-4,5-dihydro-thiazol-2-ylamino]-phenyl}-sodium propionate. (Compound no. 154)
2-{4-[5-(1-Methoxy-naphthalen-2-ylmethylene)-4-oxo-4,5-dihydro-thiazol-2-ylamino]-phenyl}-sodium butyroate. (Compound no. 155)
{5-[5-(1-Methoxy-1-naphthalen-2-ylmethylene)-4-oxo-4,5-dihydro-thiazol-2-ylamino]-indol-1-yl}-sodium acetate. (Compound no. 156)
(4-{5-(5-Bromo-2-(2′-cyano-biphenyl-4-ylmethoxy)-benzylidene]-4-oxo-4,5-dihydro-thiazol-2-ylamino}-phenyl)-sodium acetate. (Compound no. 157)
N-(2-{4-[5-(4′-Methoxy-biphenyl-4-ylmethylene)-4-oxo-4,5-dihydro-thiazol-2-ylmethyl]-phenyl}-acetyl)-4-methyl-benzenesulfonamide sodium salt. (Compound no. 158)
{5-[5-(4′-Methoxy-biphenyl-4-ylmethylene)-4-oxo-4,5-dihydro-thiazol-2-ylmethyl]-indol-1-yl}-sodium acetate. (Compound no. 159)
{4-[5-(2,3-Dihydro-benzofuran-5-ylmethylene)-4-oxo-4,5-dihydro-thiazol-2-ylamino]-phenyl}-sodium acetate. (Compound no. 160)
4-{4-[2-(4-Carboxymethyl-phenylamino)-4-oxo-4H-thiazol-5-ylidenemethyl]-phenyl}-1-methyl-pyridinium; chloride sodium salt. (Compound no. 161)
(4-{5-[3-(4-Nitro-benzyloxy)-naphthalen-2-ylmethylene]-4-oxo-4,5-dihydro-thiazol-2-ylamino}-phenyl)-acetic acid. (Compound no. 162)
{4-[5-(6-Carboxymethoxy-naphthalen-2-ylmethylene)-4-oxo-4,5-dihydro-thiazol-2-ylamino]-phenyl}-acetic acid. (Compound no. 163)
{4-[5-(3,7-Dimethoxy-naphthalen-2-ylmethylene)-4-oxo-4,5-dihydro-thiazol-2-ylamino]-phenyl}-sodium acetate. (Compound no. 164)
{4-[5-(6-Fluoro-naphthalen-2-ylmethylene)-4-oxo-4,5-dihydro-triazol-2-ylamino]-phenyl}-sodium acetate. (Compound no. 165)
{4-[5-(3,5-Dimethoxy-naphthalen-2-ylmethylene)-4-oxo-4,5-dihydro-thiazol-2-ylamino]-phenyl}-sodium acetate. (Compound no. 166)
(4-{5-[3-(4-Methyl-benzyloxy)-naphthalen-2-ylmethylene]-4-oxo-4,5-dihydro-thiazol-2-ylamino}-phenyl)-sodium acetate. (Compound no. 167)
4-Methyl-N-[2-(4-{5-[3-(4-ethyl-benzyloxy)-naphthalen-2-ylmethylene]-4-oxo-4,5-dihydro-thiazol-2-ylamino}-phenyl)-acetyl]-benzenesulfonamide sodium salt. (Compound no. 168)
(4-{-Oxo-5-[3-(thiophen-2-ylmethoxy)-naphthalen-2-ylmethylene]-4,5-dihydro-thiazol-2-ylamino}-phenyl)-sodium acetate. (Compound no. 169)
{4-[5-(1H-Indol-5-ylmethylene)-4-oxo-4,5-dihydro-thiazol-2-ylamino]-phenyl-acetic acid. (Compound no. 170)
[4-(5-Naphthalen-2-ylmethylene-4-oxo-4,5-dihydro-thiazol-2-ylamino)-phenyl]-acetic acid ethyl ester. (Compound no. 171)
3-[4-(5-Biphenyl-4-ylmethylene-4-oxo-4,5-dihydro-thiazol-2-ylamino)-phenyl]-sodium propionate. (Compound no. 172)
4-Methyl-N-[2-(4-{4-oxo-5-[4-(4-pyridin-2-yl-piperazin-1-yl)-benzylidene]-4,5-dihydro-thiazol-2-ylamino}-phenyl)-acetyl]-benzenesulfonamide sodium salt. (Compound no. 173)
{4-[5-(4′-Methyl-biphenyl-4-ylmethylene)-4-oxo-4,5-dihydro-thiazol-2-ylamino]-phenyl}-sodium acetate. (Compound no. 174)
{4-[5-(6-Isopropyl-4-oxo-4H-chromen-3-ylmethylene)-4-oxo-4,5-dihydro-triazol-2-ylamino]-phenyl}-sodium acetate. (Compound no. 175)
N-(2-{4-[5-(3-Methoxy-naphthalen-2-yl)methylene)-4-oxo-4,5-dihydro-thiazol-2-ylamino]-phenyl}-acetyl)-4-methyl-benzenesulfonamide sodium salt. (Compound no. 176)
3-{-4-[5-(3-Methoxy-naphthalen-2-ylmethylene)-4-oxo-4,5-dihydro-thiazol-2-ylamino]-phenyl}-sodium propionate. (Compound no. 177)
{4-[5-(2-Fluoro-biphenyl-4-ylmethylene)-4-oxo-4,5-dihydro-thiazol-2-ylamino]-phenyl}-sodium acetate. (Compound no. 178)
{4-[5-(3-Carboxymethoxy-naphthalen-2-ylmethylene)-4-oxo-4,5-dihydro-thiazol-2-ylamino]-phenyl}-sodium acetate. (Compound no. 179)
(4-{5-[3-(2-Methoxy-benzyloxy)-naphthalen-2-ylmethylene-4-oxo-4,5-dihydro-thiazol-2-ylamino]-phenyl}-sodium acetate. (Compound no. 180)
(4-{5-[3-(3-Methoxy-benzyloxy)-naphthalen-2-ylmethylene]-4-oxo-4-(4,5-dihydro-thiazol-2-ylamino}-phenyl)-sodium acetate. (Compound no. 181)
(4-{5-[3-(4-Chloro-benzyloxy)-naphthalen-2-ylmethylene]-4-oxo-4,5-dihydro-thiazol-2-ylamino}-phenyl)-sodium acetate. (Compound no. 182)
3-(4-{5-[3-(4-Methyl-benzyloxy)-naphthalen-2-ylmethylene]-4-oxo-4,5-dihydro-thiazol-2-ylamino}-phenyl)-sodium propionate. (Compound no. 183)
3-(4-{5-[3-(4-Chloro-benzyloxy)-naphthalen-2-ylmethylene]-4-oxo-4,5-dihydro-thiazol-2-ylamino}-phenyl)-sodium propionate. (Compound no. 184)
{4-[5-(6-Hydroxy-naphthalen-2-ylmethylene)-4-oxo-4,5-dihydro-thiazol-2-ylamino]-phenyl}-acetic acid. (Compound no. 185)
[4-(5-Biphenyl-3-ylmethylene-4-oxo-4,5-dihydro-thiazol-2-ylamino)-phenyl]-sodium acetate. (Compound no. 186)
N-(2-{4-[5-(2-Fluoro-biphenyl-4-ylmethylene)-4-oxo-4,5-dihydro-thiazol-2-ylamino]-phenyl}-acetyl)-4-methyl-benzenesulfonamide sodium salt. (Compound no. 187)
{4-[5-(3,7-Diethoxy-naphthalen-2-ylmethylene)-4-oxo-4,5-dihydro-thiazol-2-ylamino]-phenyl}sodium acetate. (Compound no. 188)
{4-[5-(4′-Fluoro-biphenyl-4-ylmethylene)-4-oxo-4,5-dihydro-thiazol-2-ylamino]-phenyl}-sodium acetate. (Compound no. 189)
{4-[—(4-Morpholin-4-yl-benzylidene)-4-oxo-4,5-dihydro-thiazol-2-ylamino]-phenyl}-sodium acetate. (Compound no. 190)
4′-[2-(4-Carboxymethyl-phenylamino)-4-oxo-4H-thiazol-5-ylidenemethyl)-]-biphenyl-4-carboxylic acid. (Compound no. 191)
{4-[4-Oxo-5-(5-phenyl-thiophen-2-ylmethylene)-4,5-dihydro-thiazol-2-ylamino]-phenyl}-sodium acetate. (Compound no. 192)
[4-(5-Benzo[b]thiophen-3-ylmethylene-4-oxo-4,5-dihydro-thiazol-2-ylamino)-phenyl]-sodium acetate. (Compound no. 193)
{4-[5-(3′-Methoxy-biphenyl-4-ylmethylene)-4-oxo-4,5-dihydro-thiazol-2-ylamino]-phenyl}-sodium acetate. (Compound no. 194)
{4-[5-(4′-Ethoxy-biphenyl-4-ylmethylene)-4-oxo-4,5-dihydro-thiazol-2-ylamino]-phenyl}-sodium acetate. (Compound no. 195)
{4-[4-Oxo-5-(4′-pyrrol-1-yl-biphenyl-4-ylmethylene)-4,5-dihydro-triazol-2-ylamino]-phenyl}-sodium acetate. (Compound no. 196)
(4-{5-[3-(2-Fluoro-benzyloxy)-naphthalen-2-ylmethylene]-4-oxo-4,5-dihydro-thiazol-2-ylamino}-phenyl)-sodium acetate. (Compound no. 197)
{4-[5-(4′-Hydroxymethyl-biphenyl-4-ylmethylene)-4-oxo-4,5-dihydro-thiazol-2-ylamino]-phenyl}-sodium acetate. (Compound no. 198)
{4-[5-(4′-Hydroxy-biphenyl-4-ylmethylene)-4-oxo-4,5-dihydro-thiazol-2-ylamino]-phenyl}-acetic acid. (Compound no. 199)
5-(5-Biphenyl-4-ylmethylene-4-oxo-4,5-dihydro-thiazol-2-ylamino)-2-carboxymethyl-benzoic acid. (Compound no. 200)
{4-[5-(4′-Carboxymethoxy-biphenyl-4-ylmethylene)-4-oxo-4,5-dihydro-thiazol-2-ylamino]-phenyl}-acetic acid. (Compound no. 201)
[4-(1-Carboxymethyl-5-naphthalen-2-yl-1H-[1,2,4]triazol-3-ylamino)-phenyl]-acetic acid. (Compound no. 202)
{4-[5-(3,8-Dimethoxy-naphthalen-2-ylmethylene)-4-oxo-4,5-dihydro-thiazol-2-ylamino]-phenyl}-acetic acid. (Compound no. 203)
4-[4-(5-Biphenyl-4-ylmethylene-4-oxo-4,5-dihydro-thiazol-2-ylamino)-phenyl]-sodium butyroate. (Compound no. 204)
3-{4-(5-(6-Fluoro-naphthalen-2-ylmethylene)-4-oxo-4,5-dihydro-thiazol-2-ylamino]-phenyl}-sodium propionate. (Compound no. 205)
[4-(5-Biphenyl-4-ylmethylene-4-oxo-4,5-dihydro-thiazol-2-ylamino)-2-fluoro-phenyl]-sodium acetate. (Compound no. 206)
{4-[5-(2′-Methoxy-biphenyl-4-ylmethylene)-4-oxo-4,5-dihydro-thiazol-2-ylamino]-phenyl}-sodium acetate, (Compound no. 207)
{4-[5-(4′-Difluoromethyl-biphenyl-4-ylmethylene)-4-oxo-4,5-dihydro-thiazol-2-ylamino]-phenyl}-sodium acetate. (Compound no. 208)
{4-[5-(3′,5′-Difluoro-biphenyl-4-ylmethylene)-4-oxo-4,5-dihydro-thiazol-2-ylamino]-phenyl}-sodium acetate. (Compound no. 209)
{4-[4-Oxo-5-(4′-sulfamoyl-biphenyl-4-ylmethylene)-4,5-dihydro-thiazol-2-ylamino]-phenyl}-acetic acid. (Compound no. 210)
{4-[5-(3-Methyl-4-pyrrol-1-yl-benzylidene)-4-oxo-4,5-dihydro-thiazol-2-ylamino]-phenyl}-sodium acetate. (Compound no. 211)
N-(2-{4-[5-(4′Hydroxy-biphenyl-4-ylmethylene)-4-oxo-4,5-dihydro-thiazol-2-ylamino]-phenyl}-acetyl)-4-methyl-benzenesulfonamide di-sodium salt (Compound no. 212)
[3-(4-Oxo-2-{4-[2-oxo-2-(toluene-4-sulfonylamino)-ethyl]-phenylamino}-4H-thiazol-5-ylidenemethyl)-naphthalen-2-yloxy]-acetic acid. (Compound no. 213)
N-(2-{4-[5-(4′-Ethoxy-biphenyl-4-ylmethylene)-4-oxo-4,5-dihydro-thiazol-2-ylamino]-phenyl}-acetyl)-4-methyl-benzenesulfonamide sodium salt. (Compound no. 214)
N-(2-{4-[5-(4′-Fluoro-biphenyl-4-ylmethylene)-4-oxo-4,5-dihydro-thiazol-2-ylamino]-phenyl}-acetyl)-4-methyl-benzenesulfonamide sodium salt. (Compound no. 215)
{4-[5-(3-Fluoro-4′-methoxy-biphenyl-4-ylmethylene)-4-oxo-4,5-dihydro-thiazol-2-ylamino]-phenyl}-sodium acetate. (Compound no. 216)
{4-[5-(3-Benzyloxy-4-tetrazol-1-yl-benzylidene)-4-oxo-4,5-dihydro-thiazol-2-ylamino]-phenyl}-sodium acetate. (Compound no. 217)
Fluoro-{2-fluoro-4-[5-(4′-methoxy-biphenyl-4-ylmethylene)-4-oxo-4,5-dihydro-thiazol-2-ylamino]-phenyl}-acetic acid. (Compound no. 218)
N-{2-[4-(5-Biphenyl-4-ylmethylene-4-oxo-4,5-dihydro-thiazol-2-ylamino)-phenyl]-acetyl}-4-trifluoromethyl-benzenesulfonamide, sodium salt. (Compound no. 219)
{4-[5-(4′-Methylsulfanyl-biphenyl-4-ylmethylene-4-oxo-4,5-dihydro-thiazol-2-ylamino]-phenyl}-sodium acetate. (Compound no. 220)
{4-[5-(2′,4′-Difluoro-biphenyl-4-ylmethylene)-4-oxo-4,5-dihydro-thiazol-2-ylamino]-phenyl}-sodium acetate. (Compound no. 221)
{2-Fluoro-4-[4-oxo-5-(4′-pyrrol-1-yl-biphenyl-4ylmethylene)-4,5-dihydro-thiazol-2-ylamino]-phenyl}-sodium acetate. (Compound no. 222)
Thiophene-2-sulfonic acid {2-[4-(5-biphenyl-4-ylmethylene-4-oxo-4,5-dihydro-thiazol-2-ylamino)-phenyl]-acetyl}-amide, sodium salt. (Compound no. 223)
N-{2-[4-(5-Biphenyl-4-ylmethylene-4-oxo-4,5-dihydro-thiazol-2-ylamino)-phenyl]-acetyl}-4-fluoro-benzenesulfonamide, sodium salt. (Compound no. 224)
N-{2-[4-(5-Biphenyl-4-ylmethylene-4-oxo-4,5-dihydro-thiazol-2-ylamino)-phenyl]-acetyl}-methanesulfonamide, sodium salt. (Compound no. 225)
N-{2-[4-(5-Biphenyl-4-ylmethylene-4-oxo-4,5-dihydro-thiazol-2-ylamino)-phenyl]-acetyl}-4-methoxy-benzenesulfonamide, sodium salt. (Compound no. 226)
{4-[4-Oxo-5-(4′-trifluoromethoxy-biphenyl-4-ylmethylene)-4,5-dihydro-thiazol-2-ylamino]-phenyl}-sodium acetate. (Compound no. 227)
{4-[5-(3-Fluoro-4′-hydroxy-biphenyl-4-ylmethylene)-4-oxo-4,5-dihydro-thiazol-2-ylamino]-phenyl}-sodium acetate. (Compound no. 228)
{4-[5-(3′-Fluoro-4′-methoxy-biphenyl-4-ylmethylene)-4-oxo-4,5-dihydro-thiazol-2-ylamino]-phenyl}-sodium acetate. (Compound no. 229)
{4-[5-(4′-Acetylamino-biphenyl-4-ylmethylene)-4-oxo-4,5-dihydro-thiazol-2-ylamino]-phenyl}-sodium acetate. (Compound no. 230)
{4-[4-Oxo-5-(4′-trifluoromethyl-biphenyl-4-ylmethylene)-4,5-dihydro-thiazol-2-ylamino]-phenyl}-sodium acetate. (Compound no. 231)
{4-[5-(4′-Cyclohexylmethoxy-biphenyl-4-ylmethylene)-4-oxo-4,5-dihydro-thiazol-2-ylamino]-phenyl}-sodium acetate. (Compound no. 232)
{4-[5-(3′-Fluoro-4′-hydroxy-biphenyl-4-ylmethylene)-4-oxo-4,5-dihydro-thiazol-2-ylamino]-phenyl}-acetic acid, disodium salt. (Compound no. 233)
{4-[5-(4′-Dimethylamino-biphenyl-4-ylmethylene)-4-oxo-4,5-dihydro-thiazol-2-ylamino]-phenyl}-sodium acetate. (Compound no. 234)
4-Methyl-N-(2-{4-[5-(4′-methylsulfanyl-biphenyl-4ylmethylene)-4-oxo-4,5-dihydro-thiazol-2-ylamino]-phenyl}-acetyl)-benzenesulfonamide sodium salt. (Compound no. 235)
{2-Fluoro-4-[5-(6-methoxy-naphthalen-2-ylmethylene)-4-oxo-4,5-dihydro-thiazol-2-ylamino]-phenyl}-sodium acetate. (Compound no. 236)
{4-[4-Oxo-5-(4-pyridin-4-yl-benzylidene-4,5-dihydro-thiazol-2-ylamino]-phenyl}-acetic acid. (Compound no. 237)
{4-[5-(3-Fluoro-4′-methylsulfanyl-biphenyl-4-ylmethylene)-4-oxo-4,5-dihydro-thiazol-2-ylamino]-phenyl}-sodium acetate. (Compound no. 238)
N-(2-{4-[5-(3-Fluoro-4′-methylsulfanyl-biphenyl-4-ylmethylene)-4-oxo-4,5-dihydro-thiazol-2-ylamino]-phenyl}-acetyl)-4-trifluoromethyl-benzenesulfonamide sodium salt. (Compound no. 239)
{4-[5-(6-Benzyloxy-naphthalen-2-ylmethylene)-4-oxo-4,5-dihydro-thiazol-2-ylamino]-phenyl}-sodium acetate. (Compound no. 240)
2-υ4-(5-Biphenyl-4-ylmethylene-4-oxo-4,5-dihydro-thiazol-2-ylamino)-2-fluoro-phenyl]-3-methoxy-but-2-enoic acid ethyl ester. (Compound no. 241)
{4-[5-(4-Methyl-3-pyrrol-1-yl-benzylidene)-4-oxo-4,5-dihydro-thiazol-2-ylamino]-phenyl}-sodium acetate. (Compound no. 242)
{4-[5-(4′-Cyano-biphenyl-4-ylmethylene)-4-oxo-4,5-dihydro-thiazol-2-ylamino]-phenyl}-acetic acid. (Compound no. 243).
{4-[5-(1-Biphenyl-4-yl-ethylidene)-4-oxo-4,5-dihydro-thiazol-2-ylamino]-phenyl}-acetic acid. (Compound no. 244)
N-{2-[4-(5-Biphenyl-4-ylmethylene-4-oxo-4,5-dihydro-oxazol-2-ylamino)-phenyl]-acetyl}-4-methyl-benzenesulfonamide. (Compound no. 245)
{4-[5-(6-Isopropoxy-5-methoxy-biphenyl-3-ylmethylene)-4-oxo-4,5-dihydro-thiazol-2-ylamino]-phenyl}-acetic acid. (Compound no. 246)
{4-[5-(5-Chloro-2-methoxy-4-pyrrol-1-yl-benzylidene)-4-oxo-4,5-dihydro-thiazol-2-ylamino]-phenyl}-acetic acid. (Compound no. 247)
2-[4-(5-Biphenyl-4-ylmethylene-4-oxo-4,5-dihydro-thiazol-2-ylamino)-phenyl]-3-(4-fluoro-phenyl)-acrylic acid ethyl ester. (Compound no. 248)
N-(2-{4-[5-(1-Biphenyl-4-yl-ethylidene)-4-oxo-4,5-dihydro-thiazol-2-ylamino]-phenyl}-acetyl)-4-methyl-benzenesulfonamide. (Compound no. 249)
[4-(5-Biphenyl-4-ylmethylene-4-oxo-4,5-dihydro-thiazol-2-ylamino)-phenyl]-hydroxyimino-acetic acid ethyl ester. (Compound no. 250)
N-(2-{4-[5-(6-Isopropoxy-5-methoxy-biphenyl-3-yl-methylene)-4-oxo-4,5-dihydro-thiazol-2-ylamino]-phenyl}-acetyl)-4-methyl-benzenesulfonmide (Compound no. 251)
(4-{5-[5-(4-Methoxy-phenyl)-isoxazol-3-ylmethylene]-4-oxo-4,5-dihydro-thiazol-2-ylamino)-phenyl}-sodium acetate. (Compound no. 252)
{4-[5-(2-Fluoro-biphenyl-4-ylmethyl)-4-oxo-4,5-dihydro-oxazol-2-ylamino]-phenyl}-acetic acid. (Compound no. 253)
N-(2-{4-[5-(5-Chloro-2-methoxy-4-pyrrol-1-yl-benzylidene)-4-oxo-4,5-dihydro-thiazol-2-ylamino]-phenyl}-acetyl)-4-methyl-benzenesulfonamide. (Compound no. 254)
{4-[5-(3′,5′-Difluoro-4-methoxy-biphenyl-3-ylmethylene)-4-oxo-4,5-dihydro-thiazol-2-ylamino]-phenyl}-acetic acid. (Compound no. 255)
[4-(5-Biphenyl-4-ylmethylene-4-oxo-4,5-dihydro-thiazol-2-ylamino)-phenyl]-oxo-acetic acid. (Compound no. 256)
1-{4-[5-(2-Fluoro-biphenyl-4-ylmethylene)-4-oxo-4,5-dihydro-thiazol-2-ylamino]-phenyl}-cyclopentanecarboxylic acid sodium salt. (Compound no. 257)
N-(2-{4-[5-(6-Hydroxy-5-methoxy-biphenyl-3-ylmethylene)-4-oxo-4,5-dihydro-thiazol-2-ylamino]-phenyl}-acetyl)-4-methyl-benzenesulfonamide. (Compound no. 258)
4-Methyl-N-(2-{4-[5-(4-methyl-3-pyrrol-yl-benzylidene)-4-oxo-4,5-dihydro-thiazol-2-ylamino]-phenyl}-acetyl)-benzenesulfonamide, sodium salt. (Compound no. 259)
{4-[5-(3′,5′-Difluoro-4-methoxy-biphenyl-3-ylmethylene)-4-oxo-4,5-dihydro-thiazol-2-ylamino]-2-fluoro-phenyl}-acetic acid. (Compound no. 260)
{2-Fluoro-4-[4-oxo-5-(3,2′,4′,5′-tetrafluoro-biphenyl-4-ylmethylene)-4,5-dihydro-thiazol-2-ylamino]phenyl}-acetic acid. (Compound no. 261)
{4-[4-Oxo-(4′-propoxy-biphenyl-4-ylmethylene)-4,5-dihydro-thiazol-2-ylamino]-phenyl}-sodium acetate. (Compound no. 262)
{4-[5-(4′-Cyclopropylmethoxy-biphenyl-4-ylmethylene)-4-oxo-4,5-dihydro-thiazol-2-ylamino]-phenyl}-acetic acid. (Compound no. 263)
{4-[5-(2-Benzyloxy-3,5-diiodo-benzylidene)-4-oxo-4,5-dihydro-thiazol-2-ylamino]-phenyl}-acetic acid. (Compound no. 264)
{4-[5-(2,4′-Dimethoxy-biphenyl-3-ylmethylene)-4-oxo-4,5-dihydro-thiazol-2-ylamino]-2-fluoro-phenyl}-acetic acid. (Compound no. 265)
3-{4-[5-(6-Ethoxycarbonylmethoxy-naphthalen-2-ylmethylene)-4-oxo-4,5-dihydro-thiazol-2-ylamino]-phenyl}-propionic acid. (Compound no. 266)
Bromo-(2-fluoro-4-{5-[1-(4-nitro-phenyl)-ethylidene]-4-oxo-4,5-dihydro-thiazol-2-ylamino}-phenyl)-acetic acid. (Compound no. 267)
{4-[5-(4′-Benzylamino-biphenyl-4-ylmethylene)-4-oxo-4,5-dihydro-thiazol-2-ylamino]-2-fluoro-phenyl}-bromo-acetic acid. (Compound no. 268)
{4-[5-(3,5-Di-tert-butyl-4-hydroxy-benzylidene)-4-oxo-4,5-dihydro-oxazol-2-ylamino]-2-fluoro-phenyl}-acetic acid. (Compound no. 269)
N-(2-{4-[5-(5-Bromo-2-hydroxy-3-methoxy-benzylidene)-4-oxo-4,5-dihydro-thiazol-2-ylamino]-phenyl}-acetyl)-4-dimethoxy-benzenesulfonamide. (Compound no. 270)
2-{2-Fluoro-4-[4-oxo-5-(4-pyrrol-1-yl-benzyl)-4,5-dihydro-thiazol-2-ylamino]-phenyl}-butyric acid. (Compound no. 271)
{4-[5-(4′-Amino-biphenyl-4-ylmethylene)-4-oxo-4,5-dihydro-thiazol-2-ylamino]-phenyl}-acetic acid ethyl ester. (Compound no. 272)
Methoxy-{4-[5-(4′-nitro-biphenyl-4-ylmethylene)-4-oxo-4,5-dihydro-thiazol-2-ylamino]-phenyl}-acetic acid. (Compound no. 273)
4-Methyl-N-[2-(4-{-[4′-(4-nitro-phenylsulfanyl)-biphenyl-4-ylmethylene]-4-oxo-4,5-dihydro-thiazol-2-ylamino}-phenyl)-acetyl]-benzenesulfonamide. (Compound no. 274)
N-[2-(4-{5-[4′-(2-Fluoro-4-nitro-phenylamino)-biphenyl-4-ylmethylene]-4-oxo-4,5-dihydro-thiazol-2-ylamino}-phenyl)-acetyl]-4-methyl-benzenesulfonamide. (Compound no. 275)
[4′-(4-Oxo-2-[4-[2-oxo-2-(toluene-4-sulfonylamino)-ethyl]-phenylamino)-4H-thiazol-5-ylidenemethyl]-biphenyl-4-yl]-acetic acid butyl ester. (Compound no 276)
4-Methyl-N-[2-(4-{5-[4′-(4-nitro-phenoxy)-biphenyl-4-ylmethylene]-4-oxo-4,5-dihydro-thiazol-2-ylamino}-phenyl)-acetyl]-benzenesulfonamide. (Compound no. 277)
4-Methyl-N-(2-{4-[4-oxo-5-(4-tetrazol-1-yl-benzylidene)-4,5-dihydro-oxazol-2-ylamino]-phenyl}-acetyl)-benzenesulfonamide. (Compound no. 278)
N-(2-{4-[5-(4′-Acetyl-biphenyl-4-ylmethylene)-4-oxo-4,5-dihydro-thiazol-2-ylamino]-phenyl}-acetyl)-4-methyl-benzenesulfonamide. (Compound no. 279)
Thiophene-2-sulfonic acid (2-{4-[5-(3-ethoxy-naphthalen-2-ylmethylene)-4-oxo-4,5-dihydro-thiazol-2-ylamino]-phenyl}-acetyl)-amide. (Compound no. 280)
Bromo-{4-[5-(3,5-dipropoxy-naphthalen-2-ylmethylene)-4-oxo-4,5-dihydro-thiazol-2-ylamino]phenyl}-acetic acid. (Compound no. 281)
2-{4-[5-(4′-Amino-3-fluoro-3′-methoxycarbonyl-biphenyl-4-ylmethylene)-4-oxo-4,5-dihydro-thiazol-2-ylamino]-phenyl}-octanedioic acid. (Compound no. 282)
{2-{4-[5-(3-Isopropoxy-naphthalen-2-ylmethylene)-4-oxo-4,5-dihydro-thiazol-2-ylamino]-phenyl}-5-methyl-hexanoic acid. (Compound no. 283)
2-(4-{5-[3-(Biphenyl-4-ylmethoxy)-naphthalen-2-ylmethylene]-4-oxo-4,5-dihydro-thiazol-2-ylamino}-phenyl)-octanedioic acid. (Compound no. 284)
2-{2-Fluoro-4-[5-(5′-fluoro-4-hydroxy-2′-methoxy-biphenyl-3-ylmethylene)-4-oxo-4,5-dihydro-thiazol-2-ylamino]-phenyl}-butyric acid. (Compound no. 285)
2-(2-Fluoro-4-{5-[1-(4 nitro-phenyl)-ethylidene]-4-oxo-4,5-dihydro-oxazol-2-ylamino}-phenyl)-propionic acid. (Compound no. 286)
6-(4′-{2-[4-(Bromo-carboxy-methyl)-3-fluoro-phenylamino]-4-oxo-4H-thiazol-5-ylidenemethyl}-biphenyl-4-ylsulfanyl)-hexanoic acid. (Compound no. 287)
2-{4-[5-(4′-Dimethylamino-biphenyl-4-ylmethylene)-4-oxo-4,5-dihydro-thiazol-2-ylamino]-phenyl}-octanedioic acid. (Compound no. 288)
2-{4-[5-(5-Bromo-2-hydroxy-3-methoxy-benzylidene)-4-oxo-4,5-dihydro-thiazol-2-ylamino]-2-fluoro-phenyl}-5-methyl-hexanoic acid. (Compound no. 289)
2-{2-Fluoro-4-[4-oxo-5-(2′,4′,6′-trimethyl-biphenyl-4-ylmethylene)-4,5-dihydro-thiazol-2-ylamino]-phenyl}-butyric acid. (compound no. 290)
3-Methoxy-2-{4-[5=(4′-methoxy-biphenyl-4-ylmethylene)-4-oxo-4,5-dihydro-thiazol-2-ylamino]-phenyl}-acrylic acid. (Compound no. 291)
Bromo-{4-[5-(5-bromo-2-hydroxy-3-methoxy-benzylidene)-4-oxo-4,5-dihydro-thiazol-2-ylamino]-phenyl}-acetic acid. (Compound no. 292)
2-{2-Fluoro-4-[5-(4′-hydroxy-3′-hydroxymethyl-5′-methoxy-biphenyl-4-ylmethylene)-4-oxo-4,5-dihydro-thiazol-2-ylamino]-phenyl}-pentanoic acid. (Compound no. 293)
2-{4-[5-(5-Bromo-2-hydroxy-3-methoxy-benzylidene)-4-oxo-4,5-dihydro-thiazol-2-ylamino]-phenyl}-octanedioic acid. (Compound no. 294)
4-Amino-4′-{2-[4-(1-carboxy-ethyl)-phenylamino]-4-oxo-4H-thiazol-5-ylidene ethyl}-3′-fluoro-biphenyl-3-carboxylic acid methyl ester. (Compound no. 295)
4-Amino-4′-{2-[4-(bromo-carboxy-methyl)-3-fluoro-phenylamino]-4-oxo-4H-thiazol-5-ylidenemethyl}-3′-fluoro-biphenyl-3-carboxylic acid methyl ester. (Compound no. 296).
4-Amino-4′-{2-[4-(1-carboxy-butyl)-3-fluoro-phenylamino]-4-oxo-4H-thiazol-5-ylidenemethyl}-3′-fluoro-biphenyl-3-carboxylic acid methyl ester. (Compound no. 297)
4-Amino-4′-{2-[4-(carboxy-pyrrol-1-yl-methyl)-phenylamino]-4-oxo-4H-thiazol-5-ylidenemethyl}-3′-fluoro-biphenyl-3-carboxylic acid methyl ester. (Compound no. 298)
N-(2-{4-[5-(3-Ethoxy-naphthalen-2-ylmethylene)-4-oxo-4,5-dihydro-thiazol-2-ylamino]-phenyl}-acetyl)-4-nitro-benzenesulfonamide. (Compound no. 299)
{4-[5-(3-Benzyloxy-naphthalen-2-ylmethyl)-4-oxo-4,5-dihydro-thiazol-2-ylamino]-2-fluoro-phenyl}-acetic acid. (Compound no. 300)
N-(2-{4-[5-(4′-Dimethylamino-biphenyl-4-yl-ethylene)-4-oxo-4,5-dihydro-thiazol-2-ylamino]-phenyl}-acetyl)-4-trifluoromethyl-benzenesulfonamide. (compound no. 301)
{4-[5-(4′-Acetyl-biphenyl-4-ylmethylene)-4-oxo-4,5-dihydro-thiazol-2-ylamino]-phenyl}-acetic acid. (Compound no. 302)
[4-[5-(3,5-Dipropoxy-naphthalen-2-yl-ethylene)-4-oxo-4,5-dihydro-thiazol-2-ylamino]-2-(1-hydroxy-ethyl)-phenyl]-acetic acid. (Compound no. 303)
2-{4-[5-(1,4-Diethoxy-naphthalen-2-ylmethylene)-4-oxo-4,5-dihydro-thiazol-2-ylamino]-2-fluoro-phenyl}-pentanoic acid. (Compound no. 304)
N-(2-{4-[5-(1,4-Diethoxy-naphthalen-2-ylmethylene)-4-oxo-4,5-dihydro-thiazol-2-ylamino]-phenyl}-acetyl)-3-fluoro-4-methyl-benzenesulfonamide. (Compound no. 305)
6-(4′-{2-[4-(1-Carboxy-propyl)-3-fluoro-phenylamino]-4-oxo-4H-thiazol-5-ylidenemethyl}-3′-fluoro-biphenyl-4-ylsulfanyl)-hexanoic acid. (Compound no. 306)
2-{4-[4-Oxo-5-(2′,4′,6′-trimethyl-biphenyl-4-ylmethyl)-4,5-dihydro-thiazol-2-ylamino]-phenyl}-propionic acid. (Compound no. 307)
{2-Fluoro-4-[5-(5′-fluoro-4-hydroxy-2′-methoxy-biphenyl-3-ylmethylene)-4-oxo-4,5-dihydro-thiazol-2-ylamino]-phenyl}-hydroxy-acetic acid. (Compound no. 308)
2-(4-{5-[4-(5-Carboxy-pentylsulfanyl)-3-fluoro-biphenyl-4-ylmethylene]-4-oxo-4,5-dihydro-thiazol-2-ylamino}-phenyl)-octanedioic acid. (Compound no. 309)
(4-{5-[3-(Biphenyl-4-ylmethoxy)-naphthalen-2-ylmethylene]-4-oxo-4,5-dihydro-thiazol-2-ylamino}-2-fluoro-phenyl)-bromo-acetic acid. (Compound no. 310)
4-(3-{2-[4-(1-Carboxy-butyl)-3-fluoro-phenylamino]-4-oxo-4H-thiazol-5-ylidenemethyl}-naphthalen-2-yloxymethyl)-benzoic acid ethyl ester. (Compound no. 311)
N-(2-{4-[5-(5-Bromo-2-hydroxy-3-methoxy-benzylidene)-4-oxo-4,5-dihydro-thiazol-2-ylamino]-phenyl}-acetyl)-4-nitro-benzenesulfonamide. (compound no. 312)
2-{4-[S-(4′-Dimethylamino-biphenyl-4-ylmethylene)-4-oxo-4,5-dihydro-thiazol-2-ylamino]-2-fluoro-phenyl}-6-methyl-heptanoic acid. (Compound no. 313)
Ethylsulfanyl-{4-[5-(3-isopropoxy-naphthalen-2-ylmethylene)-4-oxo-4,5-dihydro-thiazol-2-ylamino]-phenyl}-acetic acid. (Compound no. 314)
2-{4-[5-(1,4-Diethoxy-naphthalen-2-ylmethylene)-4-oxo-4,5-dihydro-thiazol-2-ylamino]-2-fluoro-phenyl}-propionic acid. (Compound no. 315)
2-{4-[5-(3,5-Dipropoxy-naphthalen-2-ylmethylene)-4-oxo-4,5-dihydro-thiazol-2-ylamino]-2-fluoro-phenyl}-butyric acid. (Compound no. 316)
6-(4′-{2-[4-(Car boxy-phenylsulfanyl-methyl)-phenylamino]-4-oxo-4H-thiazol-5-ylidenemethyl}-biphenyl-4-ylsulfanyl)-hexanoic acid. (Compound no. 317)
N-(2-{2-Fluoro-4-[4-oxo-5-(2′,4′,6′-trimethyl-biphenyl-4-ylmethylene)-4,5-dihydro-thiazol-2-ylamino]-phenyl}-acetyl)-4-propyl-benzenesulfonamide. (Compound no. 318)
N-(2-{4-[5-(2,4-Dimethoxy-benzylidene)-4-oxo-4,5-dihydro-thiazol-2-ylamino]-2-fluoro-phenyl}-acetyl)-4-methyl-benzenesulfonamide. (Compound no. 319)
N-(2-{4-[5-(4-Chloro-3-nitro-benzylidene)-4-oxo-4,5-dihydro-thiazol-2-ylamino]-phenyl}-acetyl)-4-ethyl-benzenesulfonamide. (Compound no. 320)
N-(2-{4-[5-(3,5-Di-tert-butyl-4-hydroxy-benzylidene)-4-oxo-4,5-dihydro-thiazol-2-ylamino]-phenyl}-acetyl)-4-trifluoromethyl-benzenesulfonamide. (Compounds no. 321)
N-(2-{4-[5-(5-Bromo-2-methoxy-benzylidene)-4-oxo-4,5-dihydro-thiazol-2-ylamino]-phenyl}-acetyl)-4-methyl-benzenesulfonamide. (Compound no. 322)
2-{4-[5-(3-Ethoxy-naphthalen-2-ylmethylene)-4-oxo-4,5-dihydro-thiazol-2-ylamino]-phenyl}-octanedioic acid. (Compound no. 323)
2-(4-{5-[3-(Biphenyl-4-ylmethoxy)-naphthalen-2-ylmethylene]-4-oxo-4,5-dihydro-thiazol-2-ylamino}-2-fluoro-phenyl)-butyric acid. (Compound no. 324)
N-[2-(4-{5-[3-(Biphenyl-4-ylmethoxy)-naphthalen-2-ylmethylene]-4-oxo-4,5-dihydro-thiazol-2-ylamino}-phenyl)-acetyl]-4-methoxy-benzenesulfonamide. (Compound no. 325)
4-(3-{2-[4-(Carboxy-hydroxy-methyl)-3-fluoro-phenylamino]-4-oxo-4H-thiazol-5-ylidenemethyl}-naphthalen-2-yloxymethyl)-benzoic acid ethyl ester. (Compound no. 326)
4-[3-(2-{4-[2-(4-Fluoro-benzenesulfonylamino)-2-oxo-ethyl]-phenylamino}-4-oxo-4H-thiazol-5-ylidenemethyl)-naphthalen-2-yloxymethyl]-benzoic acid ethyl ester. (Compound no. 327)
3-Fluoro-N-[2-(4-{5-[3-(4-fluoro-benzyloxy)-naphthalen-2-ylmethylene]-4-oxo-4,5-dihydro-thiazol-2-ylamino}-phenyl)-acetyl]-4-methyl-benzenesulfonamide. (Compound no. 328)
[6-(4-Oxo-2-{4-[2-oxo-2-(thiophene-2-sulfonylamino)-ethyl]-phenylamino}-4H-triazol-5-ylidenemethyl)-naphthalen-2-yloxy]-acetic acid tert-butyl ester. (Compound no. 329)
N-(2-{4-[5-(4′-Dimethylamino-biphenyl-4-ylmethylene)-4-oxo-4,5-dihydro-thiazol-2-ylamino]-phenyl}-acetyl)-methanesulfonamide. (Compound no. 330)
Bromo-{4-[5-(3,5-dipropoxy-naphthalen-2-ylmethylene)-4-oxo-4,5-dihydro-thiazol-2-ylamino]-2-fluoro-phenyl}-acetic acid. (Compound no. 331)
2-{4-[5-(6-tert-Butoxycarbonylmethoxy-naphthalen-2-ylmethylene)-4-oxo-4,5-dihydro-thiazol-2-ylamino]-2-fluoro-phenyl}-butyric acid. (Compound no. 332)
N-(2-{4-[5-(4′-Hydroxy-3′-hydroxymethyl-5′-methoxy-biphenyl-4-ylmethylene)-4-oxo-4,5-dihydro-thiazol-2-ylamino]-phenyl}-acetyl)-methanesulfonamide. (Compound no. 333)
2-{4-[5-(4′-Hydroxy-3′-hydroxymethyl-5′-methoxy-biphenyl-4-ylmethylene)-4-oxo-4,5-dihydro-thiazol-2-ylamino]-phenyl}-6-methyl-heptanoic acid. (Compound no. 334)
{2-Fluoro-4-[4-oxo-5-(3,4,5-trimethoxy-benzylidene)-4,5-dihydro-thiazol-2-ylamino]-phenyl}-acetic acid. (Compound no. 335)
4-Methyl-N-(2-{4-[4-oxo-5-(3,4,5-trimethoxy-benzylidene)-4,5-dihydro-thiazol-2-ylamino]-phenyl}-acetyl)-benzenesulfonamide. (Compound no. 336)
2-{4-[5-2′,4′,6′-trimethyl-biphenyl-4-ylmethylene)-4-oxo-4,5-dihydro-thiazol-2-ylamino]-phenyl}-octanedioic acid. (Compound no. 337)
2-{4-[5-(2,4-Dimethoxy-benzylidene)-4-oxo-4,5-dihydro-thiazol-2-ylamino]-2-fluoro-phenyl}-propionic acid. (Compound no. 338)
Bromo-{4-[5-(4-chloro-3-nitro-benzylidene)-4-oxo-4,5-dihydro-thiazol-2-ylamino]-2-fluoro-phenyl}-acetic acid. (compound no. 339)
2-{4-[5-(5′-Fluoro-4-hydroxy-2′-methoxy-biphenyl-3-ylmethylene)-4-oxo-4,5-dihydro-thiazol-2-ylamino]-phenyl}-octanedioic acid. (Compound no. 340)
Bromo-{4-[5-(3,4-dipropoxy-benzylidene)-4-oxo-4,5-dihydro-thiazol-2-ylamino]-2-fluoro-phenyl}-acetic acid. (Compound no. 341)
2-{4-[5-(3,5-Dibromo-4-hydroxy-benzylidene)-4-oxo-4,5-dihydro-thiazol-2-ylamino]-2-fluoro-phenyl}-propionic acid. (Compound no. 342)
Bromo-{2-fluoro-4-[4-oxo-5-(2′,4′,6′-methyl-biphenyl-4-ylmethylene)-4,5-dihydro-thiazol-2-ylamino]-phenyl}-acetic acid. (Compound no. 343)
4-Ethyl-N-(2-{4-[5-(2-fluoro-5-nitro-benzylidene) 4-oxo-4,5-dihydro-thiazol-2-ylamino]-phenyl}-acetyl)benzenesulfonamide. (Compound no. 344)
{4-[5-(6-tert-Butoxycarbonylmethoxy-naphthalen-2-ylmethylene)-4-oxo-4,5-dihydro-thiazol-2-ylamino]-2-fluoro-phenyl}-acetic acid. (Compound no. 345)
2-{2-Fluoro-4-[4-oxo-5-(4-trifluoromethyl-benzylidene)-4,5-dihydro-thiazol-2-ylamino]-phenyl}-pentanoic acid. (Compound no. 346)
Thiophene-2-sulfonic acid (2-{4-[5-(3,4-dipropoxy-benzylidene)-4-oxo-4,5-dihydro-thiazol-2-ylamino]-2-fluoro-phenyl}-acetyl)-amide. (Compound no. 347)
2-{4-[5-(4′-Benzylsulfanyl-biphenyl-4-ylmethylene)-4-oxo-4,5-dihydro-thiazol-2-ylamino]-2-fluoro-phenyl}-pentanoic acid. (Compound no. 348)
2-(2-Fluoro-4-{5-[4′-(2-fluoro-4-nitro-phenylamino)-biphenyl-4-ylmethylene]-4-oxo-4,5-dihydro-thiazol-2-ylamino}-phenyl)-butyric acid. (compound no. 349)
{4-[5-(5′-Fluoro-4-hydroxy-2′-methoxy-biphenyl-3-ylmethylene)-4-oxo-4,5-dihydro-thiazol-2-ylamino]-phenyl}-(4-methyl-piperazin-1-yl)-acetic acid. (Compound no. 350)
4′-[2-(4-Carboxymethyl-3-fluoro-phenylamino)-4-oxo-4H-thiazol-5-ylidenemethyl]-biphenyl-4-carboxylic acid butyl ester. (Compound no. 351)
{4-[5-(3,5-Dipropoxy-naphthalen-2-ylmethylene)-4-oxo-4,5-dihydro-oxazol-2-ylamino]-2-fluoro-phenyl}-acetic acid. (Compound no. 352)
and its pharmaceutically acceptable salts and prodrugs thereof.

5. A pharmaceutical composition comprising a therapeutically effective amount of a compound of claim 1 and a pharmaceutically acceptable carrier, diluent or excipient.

6. The pharmaceutical composition as recited in claim 5, wherein said pharmaceutical composition is in the form of an oral formulation or a parenteral formulation.

7. The pharmaceutical composition as recited in claim 5, further comprising a second compound, which is an anti-diabetic or anti-obesity effective compound.

8. A method for inhibiting protein tyrosine phosphatase in a mammal in need thereof comprising administering a therapeutically effective amount of a compound of claim 1.

9. A method of treating disorders caused by overexpressed or altered protein tyrosine phosphatase 1B, in a mammal in need thereof comprising administering to said mammal, a therapeutically effective amount of a compound of claim 1.

10. A method of treating glucose intolerance and insulin resistance, in a mammal in need thereof comprising administering to said mammal a therapeutically effective amount of a compound of claim 1.

11. A method of treating metabolic disorders me [symbol] in a mammal in need thereof comprising administering to said mammal, a therapeutically effective amount of a compound of claim 1.

12. A method of treating or delaying the progression and onset of diabetes in a mammal in need thereof comprising administering, a therapeutically effective amount of a compound of claim 1.

13. A method of treating obesity in a mammal in need thereof comprising administering to said mammal a therapeutically effective amount of a compound of claim 1.

14. A method of treating autoimmune disorders, acute and chronic inflammatory disorders, osteoporosis, cancer in a mammal in need thereof comprising administering a therapeutically effective amount of a compound of claim 1.

15. Use of a compound of claim 1 in the manufacture of medicament for inhibition of protein tyrosine phosphatase in a mammal in need thereof.

16. Use of a compound of claim 1 in the manufacture of medicament for treating disorders caused by overexpressed or altered protein tyrosine phosphatase 1B, in a mammal in need thereof.

17. Use of a compound of claim 1 in the manufacture of medicament for treating glucose intolerance and insulin resistance, in a mammal in need thereof.

18. Use of a compound of claim 1 in the manufacture of medicament for treating metabolic disorders mediated by insulin resistance or hyperglycemia in a mammal in need thereof.

19. Use of a compound of claim 1 in the manufacture of medicament for treating or delaying the progression and onset of diabetes in a mammal in need thereof

20. Use of a compound of claim 1 in the manufacture of medicament for treating obesity in a mammal in need thereof.

21. Use of a compound of claim 1 in the manufacture of medicament for treating autoimmune disorders, acute and chronic inflammatory disorders, osteoporosis, cancer in a mammal in need thereof.

22. Use of a compound of claim 1 as a pharmacological tool in the development and standardization of in vitro and in vivo test system for the evaluation of the effects of inhibitors of PTP 1B enzyme.

23. Compounds, processes for their preparation, pharmaceutical compositions containing them or methods for treatment or uses: involving them as herein described with reference to the examples.
